SEQUENCE LISTING (189 sequences)

SEQ ID NO: 1 - protein, 10 aa, Artificial Sequence
  Chemically Synthesized
GISLSSYGLS

SEQ ID NO: 2 - protein, 9 aa, Artificial Sequence
  Chemically Synthesized
AIGASGSAY

SEQ ID NO: 3 - protein, 3 aa, Artificial Sequence
  Chemically Synthesized
NYI

SEQ ID NO: 4 - protein, 13 aa, Artificial Sequence
  Chemically Synthesized
QASQSVYSDNRLS

SEQ ID NO: 5 - protein, 7 aa, Artificial Sequence
  Chemically Synthesized
EASKLAS

SEQ ID NO: 6 - protein, 13 aa, Artificial Sequence
  Chemically Synthesized
QGSYDCNSADCSA

SEQ ID NO: 7 - protein, 5 aa, Artificial Sequence
  Chemically Synthesized
SYGLS

SEQ ID NO: 8 - protein, 16 aa, Artificial Sequence
  Chemically Synthesized
AIGASGSAYYASWAKS

SEQ ID NO: 9 - protein, 3 aa, Artificial Sequence
  Chemically Synthesized
NYI

SEQ ID NO: 10 - protein, 13 aa, Artificial Sequence
  Chemically Synthesized
QASQSVYSDNRLS

SEQ ID NO: 11 - protein, 7 aa, Artificial Sequence
  Chemically Synthesized
EASKLAS

SEQ ID NO: 12 - protein, 13 aa, Artificial Sequence
  Chemically Synthesized
QGSYDCNSADCSA

SEQ ID NO: 13 - protein, 7 aa, Artificial Sequence
  Chemically Synthesized
GISLSSY

SEQ ID NO: 14 - protein, 5 aa, Artificial Sequence
  Chemically Synthesized
GASGS

SEQ ID NO: 15 - protein, 3 aa, Artificial Sequence
  Chemically Synthesized
NYI

SEQ ID NO: 16 - protein, 13 aa, Artificial Sequence
  Chemically Synthesized
QASQSVYSDNRLS

SEQ ID NO: 17 - protein, 7 aa, Artificial Sequence
  Chemically Synthesized
EASKLAS

SEQ ID NO: 18 - protein, 13 aa, Artificial Sequence
  Chemically Synthesized
QGSYDCNSADCSA

SEQ ID NO: 19 - protein, 8 aa, Artificial Sequence
  Chemically Synthesized
GISLSSYG

SEQ ID NO: 20 - protein, 7 aa, Artificial Sequence
  Chemically Synthesized
IGASGSA

SEQ ID NO: 21 - protein, 5 aa, Artificial Sequence
  Chemically Synthesized
KRNYI

SEQ ID NO: 22 - protein, 8 aa, Artificial Sequence
  Chemically Synthesized
QSVYSDNR

SEQ ID NO: 23 - protein, 3 aa, Artificial Sequence
  Chemically Synthesized
EAS

SEQ ID NO: 24 - protein, 13 aa, Artificial Sequence
  Chemically Synthesized
QGSYDCNSADCSA

SEQ ID NO: 25 - protein, 6 aa, Artificial Sequence
  Chemically Synthesized
SSYGLS

SEQ ID NO: 26 - protein, 12 aa, Artificial Sequence
  Chemically Synthesized
WIGAIGASGSAY

SEQ ID NO: 27 - protein, 4 aa, Artificial Sequence
  Chemically Synthesized
KRNY

SEQ ID NO: 28 - protein, 9 aa, Artificial Sequence
  Chemically Synthesized
YSDNRLSWY

SEQ ID NO: 29 - protein, 10 aa, Artificial Sequence
  Chemically Synthesized
LLIYEASKLA

SEQ ID NO: 30 - protein, 12 aa, Artificial Sequence
  Chemically Synthesized
QGSYDCNSADCS

SEQ ID NO: 31 - protein, 111 aa, Artificial Sequence
  Chemically Synthesized
QEQLKESGGGLFKPMDILTVTCTVSGISLSSYGLSWVRQAPGNGLEWIGAIGASGSAYYASWAKSRSTITRNTNLNTVTLKMTSLTAADTATYFCKRNYIWGPGTLVTVSS

SEQ ID NO: 32 - protein, 113 aa, Artificial Sequence
  Chemically Synthesized
AQVLTQTASPVSAAVGGTVTINCQASQSVYSDNRLSWYQQKPGQPPKLLIYEASKLASGVPPRFSGSGSGTQFTLAISGVQCDDAATYYCQGSYDCNSADCSAFGGGTEVVVK

SEQ ID NO: 33 - protein, 130 aa, Artificial Sequence
  Chemically Synthesized
METGLRWLLLVAVLKGVQCQEQLKESGGGLFKPMDILTVTCTVSGISLSSYGLSWVRQAPGNGLEWIGAIGASGSAYYASWAKSRSTITRNTNLNTVTLKMTSLTAADTATYFCKRNYIWGPGTLVTVSS

SEQ ID NO: 34 - protein, 135 aa, Artificial Sequence
  Chemically Synthesized
MDTRAPTQLLGLLLLWLPGATFAQVLTQTASPVSAAVGGTVTINCQASQSVYSDNRLSWYQQKPGQPPKLLIYEASKLASGVPPRFSGSGSGTQFTLAISGVQCDDAATYYCQGSYDCNSADCSAFGGGTEVVVK

SEQ ID NO: 35 - protein, 10 aa, Artificial Sequence
  Chemically Synthesized
GFSLSRYGVI

SEQ ID NO: 36 - protein, 9 aa, Artificial Sequence
  Chemically Synthesized
YISSTGSTY

SEQ ID NO: 37 - protein, 11 aa, Artificial Sequence
  Chemically Synthesized
SGLGYDGGRNI

SEQ ID NO: 38 - protein, 13 aa, Artificial Sequence
  Chemically Synthesized
QSSKSVANSNWLS

SEQ ID NO: 39 - protein, 7 aa, Artificial Sequence
  Chemically Synthesized
SASTLAS

SEQ ID NO: 40 - protein, 12 aa, Artificial Sequence
  Chemically Synthesized
AGGYSDISDIFG

SEQ ID NO: 41 - protein, 5 aa, Artificial Sequence
  Chemically Synthesized
RYGVI

SEQ ID NO: 42 - protein, 16 aa, Artificial Sequence
  Chemically Synthesized
YISSTGSTYYASWAKS

SEQ ID NO: 43 - protein, 11 aa, Artificial Sequence
  Chemically Synthesized
SGLGYDGGRNI

SEQ ID NO: 44 - protein, 13 aa, Artificial Sequence
  Chemically Synthesized
QSSKSVANSNWLS

SEQ ID NO: 45 - protein, 7 aa, Artificial Sequence
  Chemically Synthesized
SASTLAS

SEQ ID NO: 46 - protein, 12 aa, Artificial Sequence
  Chemically Synthesized
AGGYSDISDIFG

SEQ ID NO: 47 - protein, 7 aa, Artificial Sequence
  Chemically Synthesized
GFSLSRY

SEQ ID NO: 48 - protein, 5 aa, Artificial Sequence
  Chemically Synthesized
SSTGS

SEQ ID NO: 49 - protein, 11 aa, Artificial Sequence
  Chemically Synthesized
SGLGYDGGRNI

SEQ ID NO: 50 - protein, 13 aa, Artificial Sequence
  Chemically Synthesized
QSSKSVANSNWLS

SEQ ID NO: 51 - protein, 7 aa, Artificial Sequence
  Chemically Synthesized
SASTLAS

SEQ ID NO: 52 - protein, 12 aa, Artificial Sequence
  Chemically Synthesized
AGGYSDISDIFG

SEQ ID NO: 53 - protein, 8 aa, Artificial Sequence
  Chemically Synthesized
GFSLSRYG

SEQ ID NO: 54 - protein, 7 aa, Artificial Sequence
  Chemically Synthesized
ISSTGST

SEQ ID NO: 55 - protein, 13 aa, Artificial Sequence
  Chemically Synthesized
AQSGLGYDGGRNI

SEQ ID NO: 56 - protein, 8 aa, Artificial Sequence
  Chemically Synthesized
KSVANSNW

SEQ ID NO: 57 - protein, 3 aa, Artificial Sequence
  Chemically Synthesized
SAS

SEQ ID NO: 58 - protein, 12 aa, Artificial Sequence
  Chemically Synthesized
AGGYSDISDIFG

SEQ ID NO: 59 - protein, 6 aa, Artificial Sequence
  Chemically Synthesized
SRYGVI

SEQ ID NO: 60 - protein, 12 aa, Artificial Sequence
  Chemically Synthesized
FIGYISSTGSTY

SEQ ID NO: 61 - protein, 12 aa, Artificial Sequence
  Chemically Synthesized
AQSGLGYDGGRN

SEQ ID NO: 62 - protein, 9 aa, Artificial Sequence
  Chemically Synthesized
ANSNWLSWY

SEQ ID NO: 63 - protein, 10 aa, Artificial Sequence
  Chemically Synthesized
LLIYSASTLA

SEQ ID NO: 64 - protein, 11 aa, Artificial Sequence
  Chemically Synthesized
AGGYSDISDIF

SEQ ID NO: 65 - protein, 118 aa, Artificial Sequence
  Chemically Synthesized
QSMKESEGGLFKPTDTLTLTCTVSGFSLSRYGVIWVRQAPGKGLEFIGYISSTGSTYYASWAKSRSTITRNTNENTVTLKVTSLTAADTATYFCAQSGLGYDGGRNIWGPGTLVTVSS

SEQ ID NO: 66 - protein, 112 aa, Artificial Sequence
  Chemically Synthesized
AAVLTQTPSPVSAAVGGTVSISCQSSKSVANSNWLSWYQQKPGQPPKLLIYSASTLASGAPSRFKGSGSGTQFTLTISDVQCDDAATYYCAGGYSDISDIFGFGGGTEVVVK

SEQ ID NO: 67 - protein, 137 aa, Artificial Sequence
  Chemically Synthesized
METGLRWLLLVAVLKGVQCQSMKESEGGLFKPTDTLTLTCTVSGFSLSRYGVIWVRQAPGKGLEFIGYISSTGSTYYASWAKSRSTITRNTNENTVTLKVTSLTAADTATYFCAQSGLGYDGGRNIWGPGTLVTVSS

SEQ ID NO: 68 - protein, 134 aa, Artificial Sequence
  Chemically Synthesized
MDTRAPTQLLGLLLLWLPGATFAAVLTQTPSPVSAAVGGTVSISCQSSKSVANSNWLSWYQQKPGQPPKLLIYSASTLASGAPSRFKGSGSGTQFTLTISDVQCDDAATYYCAGGYSDISDIFGFGGGTEVVVK

SEQ ID NO: 69 - protein, 10 aa, Artificial Sequence
  Chemically Synthesized
GIDLSSNTVS

SEQ ID NO: 70 - protein, 9 aa, Artificial Sequence
  Chemically Synthesized
VSGYSGRPY

SEQ ID NO: 71 - protein, 7 aa, Artificial Sequence
  Chemically Synthesized
GSVNVNI

SEQ ID NO: 72 - protein, 13 aa, Artificial Sequence
  Chemically Synthesized
QSSQSVYSSNRLS

SEQ ID NO: 73 - protein, 7 aa, Artificial Sequence
  Chemically Synthesized
YTSTLAS

SEQ ID NO: 74 - protein, 13 aa, Artificial Sequence
  Chemically Synthesized
LGGYDCDIADCSA

SEQ ID NO: 75 - protein, 5 aa, Artificial Sequence
  Chemically Synthesized
SNTVS

SEQ ID NO: 76 - protein, 16 aa, Artificial Sequence
  Chemically Synthesized
VSGYSGRPYYASWAKG

SEQ ID NO: 77 - protein, 7 aa, Artificial Sequence
  Chemically Synthesized
GSVNVNI

SEQ ID NO: 78 - protein, 13 aa, Artificial Sequence
  Chemically Synthesized
QSSQSVYSSNRLS

SEQ ID NO: 79 - protein, 7 aa, Artificial Sequence
  Chemically Synthesized
YTSTLAS

SEQ ID NO: 80 - protein, 13 aa, Artificial Sequence
  Chemically Synthesized
LGGYDCDIADCSA

SEQ ID NO: 81 - protein, 7 aa, Artificial Sequence
  Chemically Synthesized
GIDLSSN

SEQ ID NO: 82 - protein, 5 aa, Artificial Sequence
  Chemically Synthesized
GYSGR

SEQ ID NO: 83 - protein, 7 aa, Artificial Sequence
  Chemically Synthesized
GSVNVNI

SEQ ID NO: 84 - protein, 13 aa, Artificial Sequence
  Chemically Synthesized
QSSQSVYSSNRLS

SEQ ID NO: 85 - protein, 7 aa, Artificial Sequence
  Chemically Synthesized
YTSTLAS

SEQ ID NO: 86 - protein, 13 aa, Artificial Sequence
  Chemically Synthesized
LGGYDCDIADCSA

SEQ ID NO: 87 - protein, 8 aa, Artificial Sequence
  Chemically Synthesized
GIDLSSNT

SEQ ID NO: 88 - protein, 7 aa, Artificial Sequence
  Chemically Synthesized
SGYSGRP

SEQ ID NO: 89 - protein, 9 aa, Artificial Sequence
  Chemically Synthesized
ARGSVNVNI

SEQ ID NO: 90 - protein, 8 aa, Artificial Sequence
  Chemically Synthesized
QSVYSSNR

SEQ ID NO: 91 - protein, 3 aa, Artificial Sequence
  Chemically Synthesized
YTS

SEQ ID NO: 92 - protein, 13 aa, Artificial Sequence
  Chemically Synthesized
LGGYDCDIADCSA

SEQ ID NO: 93 - protein, 6 aa, Artificial Sequence
  Chemically Synthesized
SSNTVS

SEQ ID NO: 94 - protein, 12 aa, Artificial Sequence
  Chemically Synthesized
WIGVSGYSGRPY

SEQ ID NO: 95 - protein, 8 aa, Artificial Sequence
  Chemically Synthesized
ARGSVNVN

SEQ ID NO: 96 - protein, 9 aa, Artificial Sequence
  Chemically Synthesized
YSSNRLSWF

SEQ ID NO: 97 - protein, 10 aa, Artificial Sequence
  Chemically Synthesized
LLMYYTSTLA

SEQ ID NO: 98 - protein, 12 aa, Artificial Sequence
  Chemically Synthesized
LGGYDCDIADCS

SEQ ID NO: 99 - protein, 112 aa, Artificial Sequence
  Chemically Synthesized
QSVEESGGRLVTPGTPLTLTCTVSGIDLSSNTVSWVRQAPGKGLEWIGVSGYSGRPYYASWAKGRFTISKTSSTVHLKITSPTAEDTATYFCARGSVNVNIWGPGTLVTVSS

SEQ ID NO: 100 - protein, 113 aa, Artificial Sequence
  Chemically Synthesized
AQVLTQTASPVSAAVGGTVTINCQSSQSVYSSNRLSWFQQKPGQPPKLLMYYTSTLASGVPSRFKGSGSGTEFTLTISGVQCDDAATYYCLGGYDCDIADCSAFGGGTEVVVK

SEQ ID NO: 101 - protein, 131 aa, Artificial Sequence
  Chemically Synthesized
METGLRWLLLVAVLKGVQCQSVEESGGRLVTPGTPLTLTCTVSGIDLSSNTVSWVRQAPGKGLEWIGVSGYSGRPYYASWAKGRFTISKTSSTVHLKITSPTAEDTATYFCARGSVNVNIWGPGTLVTVSS

SEQ ID NO: 102 - protein, 135 aa, Artificial Sequence
  Chemically Synthesized
MDTRAPTQLLGLLLLWLPGATFAQVLTQTASPVSAAVGGTVTINCQSSQSVYSSNRLSWFQQKPGQPPKLLMYYTSTLASGVPSRFKGSGSGTEFTLTISGVQCDDAATYYCLGGYDCDIADCSAFGGGTEVVVK

SEQ ID NO: 103 - protein, 10 aa, Artificial Sequence
  Chemically Synthesized
GIDLSSNTVS

SEQ ID NO: 104 - protein, 9 aa, Artificial Sequence
  Chemically Synthesized
VIGYSGNGY

SEQ ID NO: 105 - protein, 7 aa, Artificial Sequence
  Chemically Synthesized
GSVNCNI

SEQ ID NO: 106 - protein, 13 aa, Artificial Sequence
  Chemically Synthesized
QSSQSVISNNRLS

SEQ ID NO: 107 - protein, 7 aa, Artificial Sequence
  Chemically Synthesized
YASTLAS

SEQ ID NO: 108 - protein, 13 aa, Artificial Sequence
  Chemically Synthesized
LGGYDCNSADCST

SEQ ID NO: 109 - protein, 5 aa, Artificial Sequence
  Chemically Synthesized
SNTVS

SEQ ID NO: 110 - protein, 16 aa, Artificial Sequence
  Chemically Synthesized
VIGYSGNGYYASWAKG

SEQ ID NO: 111 - protein, 7 aa, Artificial Sequence
  Chemically Synthesized
GSVNCNI

SEQ ID NO: 112 - protein, 13 aa, Artificial Sequence
  Chemically Synthesized
QSSQSVISNNRLS

SEQ ID NO: 113 - protein, 7 aa, Artificial Sequence
  Chemically Synthesized
YASTLAS

SEQ ID NO: 114 - protein, 13 aa, Artificial Sequence
  Chemically Synthesized
LGGYDCNSADCST

SEQ ID NO: 115 - protein, 7 aa, Artificial Sequence
  Chemically Synthesized
GIDLSSN

SEQ ID NO: 116 - protein, 5 aa, Artificial Sequence
  Chemically Synthesized
GYSGN

SEQ ID NO: 117 - protein, 7 aa, Artificial Sequence
  Chemically Synthesized
GSVNCNI

SEQ ID NO: 118 - protein, 13 aa, Artificial Sequence
  Chemically Synthesized
QSSQSVISNNRLS

SEQ ID NO: 119 - protein, 7 aa, Artificial Sequence
  Chemically Synthesized
YASTLAS

SEQ ID NO: 120 - protein, 13 aa, Artificial Sequence
  Chemically Synthesized
LGGYDCNSADCST

SEQ ID NO: 121 - protein, 8 aa, Artificial Sequence
  Chemically Synthesized
GIDLSSNT

SEQ ID NO: 122 - protein, 7 aa, Artificial Sequence
  Chemically Synthesized
IGYSGNG

SEQ ID NO: 123 - protein, 9 aa, Artificial Sequence
  Chemically Synthesized
ARGSVNCNI

SEQ ID NO: 124 - protein, 8 aa, Artificial Sequence
  Chemically Synthesized
QSVISNNR

SEQ ID NO: 125 - protein, 3 aa, Artificial Sequence
  Chemically Synthesized
YAS

SEQ ID NO: 126 - protein, 13 aa, Artificial Sequence
  Chemically Synthesized
LGGYDCNSADCST

SEQ ID NO: 127 - protein, 6 aa, Artificial Sequence
  Chemically Synthesized
SSNTVS

SEQ ID NO: 128 - protein, 12 aa, Artificial Sequence
  Chemically Synthesized
WIGVIGYSGNGY

SEQ ID NO: 129 - protein, 8 aa, Artificial Sequence
  Chemically Synthesized
ARGSVNCN

SEQ ID NO: 130 - protein, 9 aa, Artificial Sequence
  Chemically Synthesized
ISNNRLSWL

SEQ ID NO: 131 - protein, 10 aa, Artificial Sequence
  Chemically Synthesized
LLIYYASTLA

SEQ ID NO: 132 - protein, 12 aa, Artificial Sequence
  Chemically Synthesized
LGGYDCNSADCS

SEQ ID NO: 133 - protein, 112 aa, Artificial Sequence
  Chemically Synthesized
QSVEESGGRLVTPGTPLTLTCTVSGIDLSSNTVSWVRQAPGKGLEWIGVIGYSGNGYYASWAKGRFTISKTSTTVDLKMTSPTAEDTATYFCARGSVNCNIWGPGTLVTVSS

SEQ ID NO: 134 - protein, 113 aa, Artificial Sequence
  Chemically Synthesized
AQVLTQTASPVSAAVGGTVTINCQSSQSVISNNRLSWLQQKPGQPPKLLIYYASTLASGVPSRFKGSGSGTQFTLTISGVQCDDAATYYCLGGYDCNSADCSTFGGGTEVVVK

SEQ ID NO: 135 - protein, 131 aa, Artificial Sequence
  Chemically Synthesized
METGLRWLLLVAVLKGVQCQSVEESGGRLVTPGTPLTLTCTVSGIDLSSNTVSWVRQAPGKGLEWIGVIGYSGNGYYASWAKGRFTISKTSTTVDLKMTSPTAEDTATYFCARGSVNCNIWGPGTLVTVSS

SEQ ID NO: 136 - protein, 135 aa, Artificial Sequence
  Chemically Synthesized
MDTRAPTQLLGLLLLWLPGATFAQVLTQTASPVSAAVGGTVTINCQSSQSVISNNRLSWLQQKPGQPPKLLIYYASTLASGVPSRFKGSGSGTQFTLTISGVQCDDAATYYCLGGYDCNSADCSTFGGGTEVVVK

SEQ ID NO: 137 - protein, 644 aa, Artificial Sequence
  Chemically Synthesized
MDYKDDDDKGLNDIFEAQKIEWHEGMAASPGSGSANPRKFSEKIALHTQRQAEETRAFEQLMTDLTLSRVQFQKLQQLRLTQYHGGSLPNVSQLRSSASEFQPSFHQADNVRGTRHHGLVERPSRNRFHPLHRRSGDKPGRQFDGSAFGANYSSQPLDESWPRQQPPWKDEKHPGFRLTSALNRTNSDSALHTSALSTKPQDPYGGGGQSAWPAPYMGFCDGENNGHGEVASFPGPLKEENLLNVPKPLPKQLWETKEIQSLSGRPRSCDVGGGNAFPHNGQNLGLSPFLGTLNTGGSLPDLTNLHYSTPLPASLDTTDHHFGSMSVGNSVNNIPAAMTHLGIRSSSGLQSSRSNPSIQATLNKTVLSSSLNNHPQTSVPNASALHPSLRLFSLSNPSLSTTNLSGPSRRRQPPVSPLTLSPGPEAHQGFSRQLSSTSPLAPYPTSQMVSSDRSQLSFLPTEAQAQVSPPPPYPAPQELTQPLLQQPRAPEAPAQQPQAASSLPQSDFQLLPAQGSSLTNFFPDVGFDQQSMRPGPAFPQQVPLVQQGSRELQDSFHLRPSPYSNCGSLPNTILPEDSSTSLFKDLNSALAGLPEVSLNVDTPFPLEEELQIEPLSLDGLNMLSDSSMGLLDPSVEETFRADRL

SEQ ID NO: 138 - protein, 644 aa, Artificial Sequence
  Chemically Synthesized
MDYKDDDDKGLNDIFEAQKIEWHEGMAASPGSGSANPRKFSEKIALHTQRQAEETRAFEQLMTDLTLSRVQFQKLQQLRLTQYHGGSLPNVSQLRSSASEFQPSFHQADNVRGTRHHGLVERPSRNRFHPLHRRSGDKPGRQFDGSAFGANYSSQPLDESWPRQQPPWKDEKHPGFRLTSALNRTNSDSALHTSALSTKPQDPYGGGGQSAWPAPYMGFCDGENNGHGEVASFPGPLKEENLLNVPKPLPKQLWETKEIQSLSGRPRSCDVGGGNAFPHNGQNLGLSPFLGTLNTGGSLPDLTNLHYSTPLPASLDTTDHHFGSMSVGNSVNNIPAAMTHLGIRSSSGLQSSRANPSIQATLNKTVLSSSLNNHPQTSVPNASALHPSLRLFSLSNPSLSTTNLSGPSRRRQPPVSPLTLSPGPEAHQGFSRQLSSTSPLAPYPTSQMVSSDRSQLSFLPTEAQAQVSPPPPYPAPQELTQPLLQQPRAPEAPAQQPQAASSLPQSDFQLLPAQGSSLTNFFPDVGFDQQSMRPGPAFPQQVPLVQQGSRELQDSFHLRPSPYSNCGSLPNTILPEDSSTSLFKDLNSALAGLPEVSLNVDTPFPLEEELQIEPLSLDGLNMLSDSSMGLLDPSVEETFRADRL

SEQ ID NO: 139 - protein, 644 aa, Artificial Sequence
  Chemically Synthesized
MDYKDDDDKGLNDIFEAQKIEWHEGMAASPGSGSANPRKFSEKIALHTQRQAEETRAFEQLMTDLTLSRVQFQKLQQLRLTQYHGGSLPNVSQLRSSASEFQPSFHQADNVRGTRHHGLVERPSRNRFHPLHRRSGDKPGRQFDGSAFGANYSSQPLDESWPRQQPPWKDEKHPGFRLTSALNRTNSDSALHTSALSTKPQDPYGGGGQSAWPAPYMGFCDGENNGHGEVASFPGPLKEENLLNVPKPLPKQLWETKEIQSLSGRPRSCDVGGGNAFPHNGQNLGLSPFLGTLNTGGSLPDLTNLHYSTPLPASLDTTDHHFGSMSVGNSVNNIPAAMTHLGIRSSSGLQSSRSNPSIQATLNKTVLSSSLNNHPQTSVPNASALHPSLRLFSLANPSLSTTNLSGPSRRRQPPVSPLTLSPGPEAHQGFSRQLSSTSPLAPYPTSQMVSSDRSQLSFLPTEAQAQVSPPPPYPAPQELTQPLLQQPRAPEAPAQQPQAASSLPQSDFQLLPAQGSSLTNFFPDVGFDQQSMRPGPAFPQQVPLVQQGSRELQDSFHLRPSPYSNCGSLPNTILPEDSSTSLFKDLNSALAGLPEVSLNVDTPFPLEEELQIEPLSLDGLNMLSDSSMGLLDPSVEETFRADRL

SEQ ID NO: 140 - protein, 8 aa, Artificial Sequence
  Chemically Synthesized
DYKDDDDK

SEQ ID NO: 141 - protein, 15 aa, Artificial Sequence
  Chemically Synthesized
GLNDIFEAQKIEWHE

SEQ ID NO: 142 - protein, 12 aa, Artificial Sequence
  Chemically Synthesized
GLQSSRSNPSIQ

SEQ ID NO: 143 - protein, 12 aa, Artificial Sequence
  Chemically Synthesized
  MOD_RES (7)..(7): PHOSPHORYLATION
GLQSSRSNPSIQ

SEQ ID NO: 144 - protein, 12 aa, Artificial Sequence
  Chemically Synthesized
RLFSLSNPSLST

SEQ ID NO: 145 - protein, 12 aa, Artificial Sequence
  Chemically Synthesized
  MOD_RES (6)..(6): PHOSPHORYLATION
RLFSLSNPSLST

SEQ ID NO: 146 - protein, 20 aa, Artificial Sequence
  Chemically Synthesized
GGSEGKSSGSGSESKSTGGS

SEQ ID NO: 147 - protein, 8 aa, Artificial Sequence
  Chemically Synthesized
GGGSGGGS

SEQ ID NO: 148 - protein, 12 aa, Artificial Sequence
  Chemically Synthesized
GGGSGGGSGGGS

SEQ ID NO: 149 - protein, 16 aa, Artificial Sequence
  Chemically Synthesized
GGGSGGGSGGGSGGGS

SEQ ID NO: 150 - protein, 20 aa, Artificial Sequence
  Chemically Synthesized
GGGSGGGSGGGSGGGSGGGS

SEQ ID NO: 151 - protein, 15 aa, Artificial Sequence
  Chemically Synthesized
GGGGSGGGGSGGGGS

SEQ ID NO: 152 - protein, 20 aa, Artificial Sequence
  Chemically Synthesized
GGGGSGGGGSGGGGSGGGGS

SEQ ID NO: 153 - protein, 25 aa, Artificial Sequence
  Chemically Synthesized
GGGGSGGGGSGGGGSGGGGSGGGGS

SEQ ID NO: 154 - protein, 18 aa, Artificial Sequence
  Chemically Synthesized
GSTSGSGKPGSGEGSTKG

SEQ ID NO: 155 - protein, 14 aa, Artificial Sequence
  Chemically Synthesized
IRPRAIGGSKPRVA

SEQ ID NO: 156 - protein, 15 aa, Artificial Sequence
  Chemically Synthesized
GKGGSGKGGSGKGGS

SEQ ID NO: 157 - protein, 15 aa, Artificial Sequence
  Chemically Synthesized
GGKGSGGKGSGGKGS

SEQ ID NO: 158 - protein, 15 aa, Artificial Sequence
  Chemically Synthesized
GGGKSGGGKSGGGKS

SEQ ID NO: 159 - protein, 15 aa, Artificial Sequence
  Chemically Synthesized
GKGKSGKGKSGKGKS

SEQ ID NO: 160 - protein, 15 aa, Artificial Sequence
  Chemically Synthesized
GGGKSGGKGSGKGGS

SEQ ID NO: 161 - protein, 15 aa, Artificial Sequence
  Chemically Synthesized
GKPGSGKPGSGKPGS

SEQ ID NO: 162 - protein, 20 aa, Artificial Sequence
  Chemically Synthesized
GKPGSGKPGSGKPGSGKPGS

SEQ ID NO: 163 - protein, 20 aa, Artificial Sequence
  Chemically Synthesized
GKGKSGKGKSGKGKSGKGKS

SEQ ID NO: 164 - protein, 14 aa, Artificial Sequence
  Chemically Synthesized
STAGDTHLGGEDFD

SEQ ID NO: 165 - protein, 15 aa, Artificial Sequence
  Chemically Synthesized
GEGGSGEGGSGEGGS

SEQ ID NO: 166 - protein, 15 aa, Artificial Sequence
  Chemically Synthesized
GGEGSGGEGSGGEGS

SEQ ID NO: 167 - protein, 15 aa, Artificial Sequence
  Chemically Synthesized
GEGESGEGESGEGES

SEQ ID NO: 168 - protein, 15 aa, Artificial Sequence
  Chemically Synthesized
GGGESGGEGSGEGGS

SEQ ID NO: 169 - protein, 20 aa, Artificial Sequence
  Chemically Synthesized
GEGESGEGESGEGESGEGES

SEQ ID NO: 170 - protein, 18 aa, Artificial Sequence
  Chemically Synthesized
GSTSGSGKPGSGEGSTKG

SEQ ID NO: 171 - protein, 19 aa, Artificial Sequence
  Chemically Synthesized
PRGASKSGSASQTGSAPGS

SEQ ID NO: 172 - protein, 19 aa, Artificial Sequence
  Chemically Synthesized
GTAAAGAGAAGGAAAGAAG

SEQ ID NO: 173 - protein, 19 aa, Artificial Sequence
  Chemically Synthesized
GTSGSSGSGSGGSGSGGGG

SEQ ID NO: 174 - protein, 20 aa, Artificial Sequence
  Chemically Synthesized
GKPGSGKPGSGKPGSGKPGS

SEQ ID NO: 175 - protein, 4 aa, Artificial Sequence
  Chemically Synthesized
GSGS

SEQ ID NO: 176 - protein, 10 aa, Artificial Sequence
  Chemically Synthesized
APAPAPAPAP

SEQ ID NO: 177 - protein, 20 aa, Artificial Sequence
  Chemically Synthesized
APAPAPAPAPAPAPAPAPAP

SEQ ID NO: 178 - protein, 32 aa, Artificial Sequence
  Chemically Synthesized
AEAAAKEAAAKEAAAAKEAAAAKEAAAAKAAA

SEQ ID NO: 179 - DNA, 333 bp, Artificial Sequence
  Chemically Synthesized
caggagcagttgaaggaatccgggggaggtctcttcaagccaatggatatcctgacagtcacctgcacagtctctggaatctccctcagtagctatggactgagctgggtccgccaggctccagggaacgggctggaatggatcggagccattggtgctagtggtagcgcatactacgcgagctgggcgaaaagccgatccaccatcaccagaaacaccaacctgaacacggtgactctgaaaatgaccagtctgacagccgcggacacggccacctatttctgtaaaagaaattacatctggggcccaggcaccctggtcaccgtctcctca

SEQ ID NO: 180 - DNA, 339 bp, Artificial Sequence
  Chemically Synthesized
gctcaagtgctgacccagactgcatcgcccgtgtctgcggctgtgggaggcacagtcaccatcaactgccaggccagtcagagtgtttatagtgacaaccgcttatcctggtatcaacagaaaccagggcagcctcccaagctcctgatctacgaagcatccaaactggcatctggggtcccaccgcggttcagcggcagtggatctgggacacagttcactctcgccatcagcggcgtgcagtgtgacgatgctgccacttactactgtcaaggcagttatgattgtaatagtgctgattgtagtgctttcggcggagggaccgaggtggtggtcaaa

SEQ ID NO: 181 - DNA, 354 bp, Artificial Sequence
  Chemically Synthesized
cagtcgatgaaggagtccgagggaggtctcttcaagccaacggataccctgacactcacctgcacagtctctggattctccctcagtaggtatggagtgatctgggtccgccaggctccagggaaggggctggaattcatcggatacataagtagtactggtagcacatactacgcgagctgggcgaaaagccgatccaccatcaccagaaacaccaacgagaacacggtgactctgaaagtgaccagtctgacagccgcggacacggccacctatttctgtgcgcagagtggtcttggttatgatggtggtaggaacatctggggcccaggcaccctggtcaccgtctcctcg

SEQ ID NO: 182 - DNA, 336 bp, Artificial Sequence
  Chemically Synthesized
gccgccgtgctgacccagactccatctcccgtgtctgcagctgtgggaggcacagtcagcatcagttgccagtccagtaagagtgttgctaatagcaactggctatcctggtatcagcagaaaccagggcagcctcccaagctcctgatctattctgcatccactctggcatctggggccccatcgcggttcaaaggcagtggatctgggacacagttcactctcaccatcagcgacgtgcagtgtgacgatgctgccacttactactgtgcaggcggttatagtgatattagtgatatatttggtttcggcggagggaccgaggtggtggtcaaa

SEQ ID NO: 183 - DNA, 336 bp, Artificial Sequence
  Chemically Synthesized
cagtcggtggaggagtccgggggtcgcctggtcacgcctgggacgcccctgacactcacctgcacagtctctggaatcgacctcagtagcaatacagtgagttgggtccgccaggctccagggaaggggctggagtggatcggagtcagtggttatagtggtcgtccatactacgcgagctgggcgaaaggccgattcaccatctccaaaacctcgtccacggtacatctgaagatcaccagtccgacagccgaggacacggccacctatttctgtgccagaggtagcgtgaatgtgaatatctggggcccaggcaccctggtcaccgtctcctca

SEQ ID NO: 184 - DNA, 339 bp, Artificial Sequence
  Chemically Synthesized
gcccaagtgctgacccagactgcatcccccgtgtctgcggctgttggaggcacagtcaccatcaattgccagtccagtcagagtgtttatagttcaaaccgcttatcctggtttcagcagaaaccagggcagcctcccaaactcctgatgtattatacgtccactctggcatctggggtcccatcgcggttcaaaggcagtggatctgggacagagttcactctcaccatcagcggcgtacagtgtgacgatgctgccacttactactgcctaggcggttatgattgtgatattgctgattgtagcgctttcggcggagggaccgaggtggtggtcaaa

SEQ ID NO: 185 - DNA, 336 bp, Artificial Sequence
  Chemically Synthesized
cagtcggtggaggagtccgggggtcgcctggtcacgcctgggacacccctgacactcacctgcacagtctctggaatcgacctcagtagcaatacagtgagctgggtccgccaggctccagggaaggggctggaatggatcggagtcattggttatagtggtaatggatactatgcgagctgggcgaaaggccgattcaccatctccaaaacctcgaccacggtggatctgaaaatgaccagtccgacagccgaggacacggccacctatttctgtgccagaggtagcgttaattgtaatatctggggcccaggcaccctggtcaccgtctcctca

SEQ ID NO: 186 - DNA, 339 bp, Artificial Sequence
  Chemically Synthesized
gcccaagtgctgacccagactgcatcccccgtgtctgcggctgtgggaggcacagtcaccatcaattgccagtccagtcagagtgttattagtaacaaccgcttatcctggcttcagcagaaaccagggcagcctcccaagctcctgatctattatgcatccactctggcatctggggtcccatcgcggttcaaaggcagtggatctgggacacaattcactctcaccatcagcggcgtgcagtgtgacgatgctgccacttactactgtctaggcggttatgattgtaatagtgctgattgtagcactttcggcggagggaccgaggtggtggtcaaa

SEQ ID NO: 187 - protein, 731 aa, Artificial Sequence
  Chemically Synthesized
MKHHHHHHPMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPGGSGENLYFQSGSMADGPRHLQRGPVRVGFYDIEGTLGKGNFAVVKLGRHRITKTEVAIKIIDKSQLDAVNLEKIYREVQIMKMLDHPHIIKLYQVMETKSMLYLVTEYAKNGEIFDYLANHGRLNESEARRKFWQILSAVDYCHGRKIVHRDLKAENLLLDNNMNIKIADFGFGNFFKSGELLATWCGSPPYAAPEVFEGQQYEGPQLDIWSMGVVLYVLVCGALPFDGPTLPILRQRVLEGRFRIPYFMSEDCEHLIRRMLVLDPSKRLTIAQIKEHKWMLIEVPVQRPVLYPQEQENEPSIGEFNEQVLRLMHSLGIDQQKTIESLQNKSYNHFAAIYFLLVERLKSHRSS

SEQ ID NO: 188 - protein, 120 aa, Homo sapiens
SVGNSVNNIPAAMTHLGIRSSSGLQSSRSNPSIQATLNKTVLSSSLNNHPQTSVPNASALHPSLRLFSLSNPSLSTTNLSGPSRRRQPPVSPLTLSPGPEAHQGFSRQLSSTSPLAPYPT

SEQ ID NO: 189 - protein, 120 aa, Mus musculus
SVGNSVGNLPAAMTHLGIRTSSGLQSSRSNPSIQATLSKMALSSSLKCHPQPSVANASALHPSLRLFSLSNPSLSTTNLSGPSRRRQPPVSPLTLSPGPEAHQGFSRQLSATSPLNPYPA

(19) United States
(12) Patent Application Publication
(10) Pub. No.: US 2024/0262900 A1
(43) Pub. Date: Aug. 8, 2024

(54) MATERIALS AND METHODS FOR DIFFERENTIATING CREB REGULATEDTRANSCRIPTION COACTIVATOR 3

(71) Applicants: JANSSEN BIOTECH, INC., Horsham, PA (US); The Broad Institute, Inc., Cambridge, MA (US); The General Hospital Corporation, Boston, MA (US)

(72) Inventors: Nathan Majewski, Spring House, PA (US); Ramnik Xavier, Cambridge, MA (US); Thomas Sundberg, Cambridge, MA (US); Gaochao Tian, Spring House, PA (US); Kyunghye Ahn Wisner, Spring House, PA (US); Holger Babbe, Spring House, PA (US)

(21) Appl. No.: 18/567,197

(22) PCT Filed: Jun. 6, 2022
(86) PCT No.: PCT/US22/32339

§ 371 Date: Dec. 5, 2023

Related U.S. Application Data
(60) Provisional application No. 63/197,732, filed on Jun. 7, 2021.
(60) Provisional application No. 63/197,737, filed on Jun. 7, 2021.
(60) Provisional application No. 63/197,745, filed on Jun. 7, 2021.

(51) Int. Cl. C07K 16/18; C07K 1/22; G01N 33/68

(52) U.S. Cl. CPC C07K 16/18; C07K 1/22; G01N 33/6854; G01N 2440/14

(57) ABSTRACT

The present invention provides antigen binding domains that bind phosphorylated versions of CREB-regulated transcription coactivator 3 (CRTC3), polynucleotides encoding them, vectors, host cells, and methods of making and using them.

### CROSS REFERENCE TO RELATED APPLICATIONS

This application claims priority to U.S. Provisional Application No. 63/197,732, filed Jun. 7, 2021, U.S. Provisional Application No. 63/197,737, filed Jun. 7, 2021, and U.S. Provisional Application No. 63/197,745, filed Jun. 7, 2021, each of which is hereby incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “206389-0045-00WO_Sequence_Listing” having a creation date of May 26, 2022, and having a size of 82,614 bytes. The sequence listing submitted via EFS-Web is part of the specification and is herein incorporated by reference in its entirety.

### TECHNICAL FIELD

The invention provides antigen binding domains that bind phosphorylated versions of CREB-regulated transcription coactivator 3 (CRTC3), polynucleotides encoding them, vectors, host cells, methods of making and using them.

### BACKGROUND

A protein kinase is a protein that catalyzes the transfer of phosphate groups to other protein or organic molecule substrates. The Salt-Inducible Kinases (SIKs) are Ser/Thr kinases members of the Adenosine Monophosphate-Activated Kinase (AMPK) subfamily of kinases with three known isoforms, i.e., SIK1, SIK2 (QIK), and SIK3 (QSK) (Bright N J, et al., Acta physiologica, 2009, 196(1): 15-26).

CREB Regulated Transcription Coactivator (CRTC3)-3 (previously known as TORC-3) is a member of the CREB-regulated transcription coactivator gene family and represents a well-known SIK target protein (Altarejos J Y, et al., Nat Rev Mol Cell Biol, 2011, 12(3): 141-51; Clark K, et al., PNAS USA, 2012, 109(42): 16986-9; Sonntag T, et al., PloS one, 2017, 12(2): e0173013). CRTC proteins have been shown to act in concert with and as coactivators for the cAMP responsive element binding protein (CREB)-1 transcription factor, a member of the leucine zipper family of DNA binding proteins, to increase CREB activity following their association with residues in the basic leucine zipper domain (Bittinger M A, et al., Curr Biol, 2004, 14(23): 2156-61; Conkright M D, et al., Molecular cell, 2003, 12(2): 413-23; Iourgenko V, et al., PNAS USA, 2003, 100(21): 12147-52; Screaton R A, et al., Cell, 2004, 119(1): 61-74). For example, CREB is known to facilitate target gene activation in response to cyclic adenosine monophosphate (cAMP) and calcium signals (Bonni A, et al., Mol Cell Neurosci, 1995, 6(2): 168-83), a process for which CRTC proteins are critically required. The regulation of CRTC activity primarily occurs through nucleo-cytoplasmic shuttling in response to phosphorylation and dephosphorylation at specific residues within the protein (Screaton R A, et al., Cell, 2004, 119(1): 61-74). A specific example where this process is engaged is the cellular response to fasting and feeding signals on the expression of metabolic programs in insulin-sensitive tissues (Altarejos J Y, et al., Nat Rev Mol Cell Biol, 2011, 12(3): 141-51; Koo S-H, et al., Nature, 2005, 437(7062): 1109-11). In the basal state, cellular CRTCs are sequestered in the cytoplasm through phosphorylation-dependent interactions with 14-3-3 proteins. Following exposure to cAMP and calcium, CRTC proteins are dephosphorylated by cellular phosphatases, e.g. calcium-dependent calcineurin, which results in their dissociation from 14-3-3-proteins and nuclear entry (Bittinger M A, et al., Curr Biol, 2004, 14(23): 2156-61; Screaton R A, et al., Cell, 2004, 119(1): 61-74). The CRTCs subsequently bind to CREB over relevant promoters triggering gene transcription (Altarejos J Y, et al., Nat Rev Mol Cell Biol, 2011, 12(3): 141-51). This process is at least partially reversible as nuclear SIK2 has been shown to stimulate CRTC2 phosphorylation triggering its relocation to the cytoplasm, thereby suppressing the gluconeogenesis program in the liver (Dentin R, et al., Nature, 2007, 449(7160): 366-9).

CRTC proteins are not exclusive substrates of the SIKs as e.g. the energy-sensing 5′ AMPK has been shown to promote phosphorylation and nuclear accumulation of CRTC2 (Altarejos J Y, et al., Nat Rev Mol Cell Biol, 2011, 12(3): 141-51; Koo S-H, et al., Nature, 2005, 437(7062): 1109-11; Jiang S-J, et al., World journal of gastroenterology, 2015, 21(25): 7777-85). Likewise, AMPK-family member MAP/microtubule affinity-regulating kinase (MARK)-2 was shown to block CRTC2:CREB activity (Jansson D, et al., PNAS USA, 2008, 105(29): 10161-6).

The CRTC3 isoform has been shown to be an important regulator of lipid metabolism in metabolic tissue. Mice genetically deficient in CRTC3 remain lean even under a high-fat diet as fat burning is increased. CRTC3 was shown to promote obesity in part by attenuating catecholamine signaling in adipose tissue (Song Y, et al., Nature, 2010, 468(7326): 933-9). In another example, in certain myeloid cells under basal conditions, the SIKs phosphorylate CRTC3 at the serine residues 62, 162, 329 and 370 which results in its 14-3-3 protein-dependent cytosolic sequestration (Clark K, et al., PNAS USA, 2012, 109(42): 16986-9; MacKenzie K F, et al., Journal of immunology, 2013, 190(2): 565-77; Walkinshaw D R, et al., The Journal of biological chemistry, 2013, 288(13): 9345-62). Following a rise in cytoplasmic cAMP levels, e.g. secondary to prostaglandin E 2 and 4 receptor stimulation, or through the adenylyl cyclase activator Forskolin, or through small molecule SIK inhibitors, SIK activity is inhibited in a Protein Kinase A-dependent manner allowing cellular phosphatases to dephosphorylate CRTC3 enabling its entry into the nucleus and stimulating the transcription of the CREB target IL-10 (Clark K, et al., PNAS USA, 2012, 109(42): 16986-9; Sonntag T, et al., PloS one, 2017, 12(2): e0173013; MacKenzie K F, et al., Journal of immunology, 2013, 190(2): 565-77).

Small molecule protein kinase inhibitors are useful for treating disease such as cancer, immune disorders, and disorders of the musculoskeletal system. A need for small molecule kinase inhibitors that selectively inhibit SIK1, SIK2 and/or SIK3 has been identified and such inhibitors are being developed (Wein M N, et al., Trends in endocrinology and metabolism: TEM, 2018, 29(10): 723-35). However, there is currently a lack of simple, sensitive, and specific assays that indicate SIK activity.

To study SIK biology, for example after inhibition of enzyme function by chemical inhibition, the availability of simple assays that indicate enzymatic activity with high specificity for the pathway and that can be performed in laboratories equipped to study molecular biology, is essential. Techniques able to indicate the relative quantities of fractions of the SIK substrate CRTC3 (Altarejos J Y, et al., Nat Rev Mol Cell Biol, 2011, 12(3): 141-51; Clark K, et al., PNAS USA, 2012, 109(42): 16986-9; Sonntag T, et al., PloS one, 2017, 12(2): e0173013) that are phosphorylated at specific sites are suitable to serve this purpose. Mass spectroscopy has been shown to detect phosphorylated CRTC species as well (Clark K, et al., PNAS USA, 2012, 109(42): 16986-9; MacKenzie K F, et al., Journal of immunology, 2013, 190(2): 565-77) but this technique requires specialized equipment. The discrimination of phosphorylated versus unphosphorylated CRTC3 protein has been attempted by immunoblotting through detection of total protein using an antibody recognizing CRTC3 irrespective of its phosphorylation state (Patel K, et al., Nature communication, 2014, 5: 4535). Treatment of cells with a small molecule SIK inhibitor caused a band-mobility shift of the CRTC3 signal in protein lysates such that a dose-dependent increase in a faster migrating form of unphosphorylated CRTC3 with a reciprocal reduction in the slower migrating phosphorylated form could be observed (Patel K, et al., Nature communication, 2014, 5: 4535). However, this technique is limited by a lack of specificity as phosphorylated CRTC protein is not directly measured. Poor quantifiability and the requirement for conditions conducive to high-resolution immunoblotting are further limitations.

Thus, there is a need in the art for compositions and methods to specifically detect phosphorylated CRTC3. The present invention satisfies this unmet need.

### SUMMARY

In one embodiment, the invention provides an isolated protein comprising an antigen binding domain that binds phosphorylated CREB Regulated Transcription Coactivator 3 (CRTC3), wherein the CRTC3 comprises at least one phosphorylated site. In one embodiment, at least one phosphorylated site is serine 329. In one embodiment, at least one phosphorylated site is serine 370. In one embodiment, at least one phosphorylated site is serine 62. In one embodiment, at least one phosphorylated site is serine 162.

In one embodiment, the present invention relates to an isolated protein comprising an antigen binding domain that binds phosphorylated CREB Regulated Transcription Coactivator 3 (CRTC3), wherein the antigen binding domain that binds phosphorylated CRTC3 comprises at least one complementarity determining region (CDR) selected from the group consisting of a heavy chain complementarity determining region (HCDR) 1, a HCDR2, a HCDR3, a light chain complementarity determining region (LCDR) 1, a LCDR2 and a LCDR 3, wherein: the HCDR1 is SEQ ID NO: 1, 7, 13, 19, 25, 35, 41, 47, 53, 59, 69, 75, 81, 87, 93, 103, 109, 115, 121, or 128; the HCDR2 is SEQ ID NO: 2, 8, 14, 20, 26, 36, 42, 48, 54, 60, 70, 76, 82, 88, 94, 104, 110, 116, 122, or 128; the HCDR3 is SEQ ID NO: 3, 9, 15, 21, 27, 37, 43, 49, 55, 61, 71, 77, 83, 89, 95, 105, 111, 117, 123, or 129; the LCDR1 is SEQ ID NO: 4, 22, 28, 38, 56, 62, 72, 90, 96, 106, 124, or 130; the LCDR2 is SEQ ID NO: 5, 23, 29, 39, 57, 63, 73, 91, 97, 107, 125, or 131; and the LCDR3 is SEQ ID NO: 6, 24, 30, 40, 58, 64, 74, 92, 98, 108, 126, or 132.

In one embodiment, the antigen binding domain of the isolated protein binds phosphorylated serine 370 (pSer370) of CRTC3, and: the HCDR1 is SEQ ID NO: 1, 7, 13, 19, 25, 35, 41, 47, 53, or 59; the HCDR2 is SEQ ID NO: 2, 8, 14, 20, 26, 36, 42, 48, 54, or 60; the HCDR3 is SEQ ID NO: 3, 9, 15, 21, 27, 37, 43, 49, 55, or 61; the LCDR1 is SEQ ID NO: 4, 22, 28, 38, 56, or 62; the LCDR2 is SEQ ID NO: 5, 23, 29, 39, 57, or 63; and the LCDR3 is SEQ ID NO: 6, 24, 30, 40, 58, or 64.

In one embodiment, the antigen binding domain of the isolated protein comprises: a HCDR1 of SEQ ID NO: 1, 7, 13, 19 or 25; a HCDR2 of SEQ ID NO: 2, 8, 14, 20 or 26; a HCDR3 of SEQ ID NO: 3, 9, 15, 21 or 27; a LCDR1 of SEQ ID NO: 4, 22, or 28; a LCDR2 of SEQ ID NO: 5, 23, or 29; and a LCDR3 of SEQ ID NO: 6, 24, or 30.

In one embodiment, the antigen binding domain of the isolated protein comprises a heavy chain variable region (VH) and light chain variable region (VL), the VH comprises an amino acid sequence at least 95% identical to the amino acid sequence of SEQ ID NO: 31, and the VL comprises an amino acid sequence at least 95% identical to the amino acid sequence of SEQ ID NO: 32.

In one embodiment, the antigen binding domain of the isolated protein comprises a heavy chain (HC) and light chain (LC), the HC comprises an amino acid sequence at least 95% identical to the amino acid sequence of SEQ ID NO: 33, and the LC comprises an amino acid sequence at least 95% identical to the amino acid sequence of SEQ ID NO: 34.

In one embodiment, the antigen binding domain of the isolated protein comprises: a HCDR1 of SEQ ID NO: 35, 41, 47, 53 or 59; a HCDR2 of SEQ ID NO: 36, 42, 48, 54 or 60; a HCDR3 of SEQ ID NO: 37, 43, 49, 55 or 61; a LCDR1 of SEQ ID NO: 38, 56, or 62; a LCDR2 of SEQ ID NO: 39, 57, or 63; and a LCDR3 of SEQ ID NO: 40, 58, or 64.

In one embodiment, the antigen binding domain of the isolated protein comprises a heavy chain variable region (VH) and light chain variable region (VL), the VH comprises an amino acid sequence at least 95% identical to the amino acid sequence of SEQ ID NO: 65, and the VL comprises an amino acid sequence at least 95% identical to the amino acid sequence of SEQ ID NO: 66.

In one embodiment, the antigen binding domain of the isolated protein comprises a heavy chain (HC) and light chain (LC), the HC comprises an amino acid sequence at least 95% identical to the amino acid sequence of SEQ ID NO: 67, and the LC comprises an amino acid sequence at least 95% identical to the amino acid sequence of SEQ ID NO: 68.

In one embodiment, the antigen binding domain of the isolated protein binds phosphorylated serine 329 (pSer329) of CRTC3, and: the HCDR1 is SEQ ID NO: 69, 75, 81, 87, 93, 103, 109, 115, 121, or 128; the HCDR2 is SEQ ID NO: 70, 76, 82, 88, 94, 104, 110, 116, 122, or 128; the HCDR3 is SEQ ID NO: 71, 77, 83, 89, 95, 105, 111, 117, 123, or 129; the LCDR1 is SEQ ID NO: 72, 90, 96, 106, 124, or 130; the LCDR2 is SEQ ID NO: 73, 91, 97, 107, 125, or 131; and the LCDR3 is SEQ ID NO: 74, 92, 98, 108, 126, or 132.

In one embodiment, the antigen binding domain of the isolated protein comprises: a HCDR1 of SEQ ID NO: 69, 75, 81, 87, or 93; a HCDR2 of SEQ ID NO: 70, 76, 82, 88, or 94; a HCDR3 of SEQ ID NO: 71, 77, 83, 89, or 95; a LCDR1 of SEQ ID NO: 72, 90, or 96; a LCDR2 of SEQ ID NO: 73, 91, or 97; and a LCDR3 of SEQ ID NO: 74, 92, or 98.

In one embodiment, the antigen binding domain of the isolated protein comprises a heavy chain variable region (VH) and light chain variable region (VL), the VH comprises an amino acid sequence at least 95% identical to the amino acid sequence of SEQ ID NO: 99, and the VL comprises an amino acid sequence at least 95% identical to the amino acid sequence of SEQ ID NO: 100.

In one embodiment, the antigen binding domain of the isolated protein comprises a heavy chain (HC) and light chain (LC), the HC comprises an amino acid sequence at least 95% identical to the amino acid sequence of SEQ ID NO: 101, and the LC comprises an amino acid sequence at least 95% identical to the amino acid sequence of SEQ ID NO: 102.

In one embodiment, the antigen binding domain of the isolated protein comprises: a HCDR1 of SEQ ID NO: 103, 109, 115, 121, or 127; a HCDR2 of SEQ ID NO: 104, 110, 116, 122, or 128; a HCDR3 of SEQ ID NO: 105, 111, 117, 123, or 129; a LCDR1 of SEQ ID NO: 106, 124, or 130; a LCDR2 of SEQ ID NO: 107, 125, or 131; and a LCDR3 of SEQ ID NO: 108, 126, or 132.

In one embodiment, the antigen binding domain of the isolated protein comprises a heavy chain variable region (VH) and light chain variable region (VL), the VH comprises an amino acid sequence at least 95% identical to the amino acid sequence of SEQ ID NO: 133, and the VL comprises an amino acid sequence at least 95% identical to the amino acid sequence of SEQ ID NO: 134.

In one embodiment, the antigen binding domain of the isolated protein comprises a heavy chain (HC) and light chain (LC), the HC comprises an amino acid sequence at least 95% identical to the amino acid sequence of SEQ ID NO: 135, and the LC comprises an amino acid sequence at least 95% identical to the amino acid sequence of SEQ ID NO: 136.

In one embodiment, the present invention relates to a pharmaceutical composition comprising any one of the isolated proteins that binds phosphorylated CRTC3 described herein and a pharmaceutically acceptable carrier.

In one embodiment, the present invention relates to a polynucleotide encoding any one of the isolated proteins that binds phosphorylated CRTC3 described herein. In one embodiment, the present invention relates to a vector comprising the polynucleotide encoding any one of the isolated proteins that binds phosphorylated CRTC3 described herein. In one embodiment, the present invention relates to a host cell comprising the vector comprising the polynucleotide encoding any one of the isolated proteins that binds phosphorylated CRTC3 described herein.

In one embodiment, the present invention relates to a method of producing any one of the isolated proteins that binds phosphorylated CRTC3 described herein, comprising culturing a host cell comprising the vector comprising the polynucleotide encoding any one of the isolated proteins that binds phosphorylated CRTC3 in conditions the promote expression of the protein, and isolating the protein produced by the host cell.

In one embodiment, the present invention relates to a method of administering an isolated protein to a subject, the method comprising administering a pharmaceutical composition comprising an isolated protein, wherein said isolated protein comprises an antigen binding domain that binds phosphorylated CREB Regulated Transcription Coactivator 3 (CRTC3), wherein the antigen binding domain that binds phosphorylated CRTC3 comprises at least one complementarity determining region (CDR) selected from the group consisting of a heavy chain complementarity determining region (HCDR) 1, a HCDR2, a HCDR3, a light chain complementarity determining region (LCDR) 1, a LCDR2 and a LCDR 3, wherein: the HCDR1 is SEQ ID NO: 1, 7, 13, 19, 25, 35, 41, 47, 53, 59, 69, 75, 81, 87, 93, 103, 109, 115, 121, or 128; the HCDR2 is SEQ ID NO: 2, 8, 14, 20, 26, 36, 42, 48, 54, 60, 70, 76, 82, 88, 94, 104, 110, 116, 122, or 128; the HCDR3 is SEQ ID NO: 3, 9, 15, 21, 27, 37, 43, 49, 55, 61, 71, 77, 83, 89, 95, 105, 111, 117, 123, or 129; the LCDR1 is SEQ ID NO: 4, 22, 28, 38, 56, 62, 72, 90, 96, 106, 124, or 130; the LCDR2 is SEQ ID NO: 5, 23, 29, 39, 57, 63, 73, 91, 97, 107, 125, or 131; and the LCDR3 is SEQ ID NO: 6, 24, 30, 40, 58, 64, 74, 92, 98, 108, 126, or 132.

In one embodiment, the pharmaceutical composition of the method of administering an isolated protein to a subject further comprises a pharmaceutically acceptable carrier. In one embodiment of the method, the subject has one or more diseases or disorders. In one embodiment of the method, the pharmaceutical composition is administered in a therapeutically effective amount to treat or prevent the one or more diseases or disorders. In one embodiment of the method, the one or more diseases or disorders comprises one or more selected from the group consisting of: Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease and an ulcerative colitis. In one embodiment of the method, the one or more diseases or disorders comprises one or more selected from the group consisting of: an ankylosing spondylitis, an atherosclerosis, an autoimmune diabetes, a primary biliary cholangitis, an inflammatory bowel disease (IBD), a systemic lupus erythematosus, a multiple sclerosis, a psoriasis, an arthritis, Sjogren's syndrome, a transplantation, a graft versus host disease (GVHD), a type I diabetes, a rheumatoid arthritis, a juvenile arthritis, a psoriatic arthritis, Celiac Disease, Crohn's disease and an ulcerative colitis.

In one embodiment, the present invention relates to a method of differentiating between phosphorylated and unphosphorylated CRTC3 in a sample, comprising: obtaining the sample; contacting the sample with an isolated protein comprising an antigen binding domain that binds phosphorylated CRTC3; and detecting the presence or absence of bound phosphorylated CRTC3, thereby determining that the CRTC3 in the sample is phosphorylated or unphosphorylated.

In one embodiment, the present invention relates to a method of detecting phosphorylated CRTC3 in a sample, comprising: obtaining the sample; contacting the sample with an isolated protein comprising an antigen binding domain that binds phosphorylated CRTC3; and detecting the bound phosphorylated CRTC3 in the sample, thereby determining the level of phosphorylated CRTC3.

In one embodiment of the method of detecting phosphorylated CRTC3, the antigen binding domain that binds phosphorylated CRTC3 comprises at least one complementarity determining region (CDR) selected from the group consisting of a heavy chain complementarity determining region (HCDR) 1, a HCDR2, a HCDR3, a light chain complementarity determining region (LCDR) 1, a LCDR2 and a LCDR 3, wherein: the HCDR1 is SEQ ID NO: 1, 7, 13, 19, 25, 35, 41, 47, 53, 59, 69, 75, 81, 87, 93, 103, 109, 115, 121, or 128; the HCDR2 is SEQ ID NO: 2, 8, 14, 20, 26, 36, 42, 48, 54, 60, 70, 76, 82, 88, 94, 104, 110, 116, 122, or 128; the HCDR3 is SEQ ID NO: 3, 9, 15, 21, 27, 37, 43, 49, 55, 61, 71, 77, 83, 89, 95, 105, 111, 117, 123, or 129; the LCDR1 is SEQ ID NO: 4, 22, 28, 38, 56, 62, 72, 90, 96, 106, 124, or 130; the LCDR2 is SEQ ID NO: 5, 23, 29, 39, 57, 63, 73, 91, 97, 107, 125, or 131; and the LCDR3 is SEQ ID NO: 6, 24, 30, 40, 58, 64, 74, 92, 98, 108, 126, or 132.

In one embodiment, the method of detecting phosphorylated CRTC3 further comprises: contacting the sample with an isolated protein comprising an antigen binding domain that binds to total CRTC3; detecting the bound total CRTC3 in the sample, thereby determining the level of total CRTC3; and dividing the level of phosphorylated CRTC3 by the level of total CRTC3, thereby determining the proportion of phosphorylated CRTC3 in the sample.

In one embodiment, the present invention relates to a method of screening one or more agents that modulates the phosphorylation state of CRTC3, comprising: obtaining a composition, or one or more cells comprising CRTC3 and one or more proteins that modulates the phosphorylation state of CRTC3; contacting the composition or the one or more cells with an agent; and detecting the phosphorylation state of CRTC3 according to the methods described herein.

In one embodiment of the method of screening one or more agents, the one or more agents modulates the activity of one or more proteins that modulates the phosphorylation state of CRTC3. In one embodiment of the method, the one or more proteins increases the phosphorylation of CRTC3. In one embodiment of the method, the one or more proteins that increases the phosphorylation of CRTC3 comprises a kinase. In one embodiment of the method, the one or more proteins decreases the phosphorylation of CRTC3. In one embodiment of the method, the one or more proteins that decreases the phosphorylation of CRTC3 comprises a phosphatase.

In one embodiment, the method of screening one or more agents further comprises comparing the level of bound phosphorylated CRTC3 of the composition or the one or more cells with the level of bound phosphorylated CRTC3 of a comparator, wherein: an increase in phosphorylation relative to said comparator indicates that said one or more agents is an enhancer of CRTC3 phosphorylation; and a decrease in phosphorylation relative to said comparator indicates that said one or more agents is an inhibitor of CRTC3 phosphorylation.

In one embodiment, the present invention relates to a method of determining the effect of one or more therapeutics that modulates the level of phosphorylation of CRTC3 in a subject, comprising: administering the one or more therapeutics to the subject; obtaining a sample from the subject; contacting the sample with the antigen binding domain that binds phosphorylated CRTC3; and detecting the level of bound phosphorylated CRTC3 in the sample of the subject as compared to the level of bound phosphorylated CRTC3 of a comparator.

In one embodiment of the method of determining the effect of one or more therapeutics that modulates the level of phosphorylation of CRTC3, an elevated level of bound phosphorylated CRTC3 in the sample of the subject as compared to the level of bound phosphorylated CRTC3 of a comparator indicates one or more selected from the group consisting of: 1) that said one or more therapeutics is efficacious for elevating the level of phosphorylated CTRC3 in a subject and 2) that the concentration of the one or more therapeutics is therapeutically effective. In one embodiment of the method, no detection of an elevated level of bound phosphorylated CRTC3 in the sample of the subject as compared to the level of bound phosphorylated CRTC3 of a comparator indicates one or more selected from the group consisting of: 1) that said one or more therapeutics is not efficacious for elevating the level of phosphorylated CTRC3 in a subject and 2) that the concentration of the one or more therapeutics is not therapeutically effective; and the subject is administered one or more selected from the group consisting of: 1) one or more additional therapeutics that elevates the level of phosphorylation of CRTC3 and 2) an increased concentration of the one or more therapeutics.

In one embodiment of the method of determining the effect of one or more therapeutics that modulates the level of phosphorylation of CRTC3, a depressed level of bound phosphorylated CRTC3 in the sample of the subject as compared to the level of bound phosphorylated CRTC3 of a comparator indicates one or more selected from the group consisting of: 1) that said one or more therapeutics is efficacious for depressing the level of phosphorylated CTRC3 in a subject and 2) that the concentration of the one or more therapeutics is therapeutically effective. In one embodiment of the method, no detection of a depressed level of bound phosphorylated CRTC3 in the sample of the subject as compared to the level of bound phosphorylated CRTC3 of a comparator indicates one or more selected from the group consisting of: 1) that said one or more therapeutics is not efficacious for depressing the level of phosphorylated CTRC3 in a subject and 2) that the concentration of the one or more therapeutics is not therapeutically effective; and the subject is administered one or more selected from the group consisting of: 1) one or more additional therapeutics that depresses the level of phosphorylation of CRTC3 and 2) an increased concentration of the one or more therapeutics.

In one embodiment, the present invention relates to a composition comprising protein means for binding to phosphorylated CREB Regulated Transcription Coactivator 3 (CRTC3). In one embodiment, said phosphorylated CRTC3 comprises phosphorylated serine 370 (pSer370). In one embodiment, said phosphorylated CRTC3 comprises phosphorylated serine 329 (pSer329). In one embodiment, said phosphorylated CRTC3 comprises phosphorylated serine 62 (pSer62). In one embodiment, said phosphorylated CRTC3 comprises phosphorylated serine 162 (pSer162).

In one embodiment, the present invention relates to a pharmaceutical composition comprising protein means for binding to phosphorylated CRTC3 and a pharmaceutically acceptable carrier.

In one embodiment, the present invention relates to a composition comprising a polynucleotide encoding protein means for binding to phosphorylated CREB Regulated Transcription Coactivator 3 (CRTC3).

In one embodiment, the present invention relates to a method of producing protein means for binding to phosphorylated CREB Regulated Transcription Coactivator 3 (CRTC3), comprising culturing a host cell in conditions the promote expression of the protein means, and isolating the protein means produced by the host cell.

In one embodiment, the present invention relates to a method comprising administering to a subject a composition comprising protein means for binding to phosphorylated CREB Regulated Transcription Coactivator 3 (CRTC3). In one embodiment of the method, the subject has one or more diseases or disorders. In one embodiment of the method, the composition is a pharmaceutical composition and is administered in a therapeutically effective amount to treat or prevent the one or more diseases or disorders. In one embodiment of the method, the one or more diseases or disorders comprises one or more selected from the group consisting of: Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease and an ulcerative colitis. In one embodiment of the method, the one or more diseases or disorders comprises one or more selected from the group consisting of: an ankylosing spondylitis, an atherosclerosis, an autoimmune diabetes, a primary biliary cholangitis, an inflammatory bowel disease (IBD), a systemic lupus erythematosus, a multiple sclerosis, a psoriasis, an arthritis, Sjogren's syndrome, a transplantation, a graft versus host disease (GVHD), a type I diabetes, a rheumatoid arthritis, a juvenile arthritis, a psoriatic arthritis, Celiac Disease, Crohn's disease and an ulcerative colitis.

In one embodiment, the present invention relates to a method of detecting phosphorylated CRTC3 in a sample, comprising: obtaining the sample; contacting the sample with a composition comprising protein means for binding to phosphorylated CREB Regulated Transcription Coactivator 3 (CRTC3); and detecting the bound phosphorylated CRTC3 in the sample, thereby determining the level of phosphorylated CRTC3.

In one embodiment, the method of detecting phosphorylated CRTC3 further comprises: contacting the sample with a composition comprising protein means that binds to total CRTC3; detecting the bound total CRTC3 in the sample, thereby determining the level of total CRTC3; and dividing the level of phosphorylated CRTC3 by the level of total CRTC3, thereby determining the proportion of phosphorylated CRTC3 in the sample.

In one embodiment, the present invention relates to a method of screening one or more agents that modulates the phosphorylation state of CRTC3, comprising: obtaining a composition, or one or more cells comprising CRTC3 and one or more proteins that modulates the phosphorylation state of CRTC3; contacting the composition or the one or more cells with one or more agents; and detecting the phosphorylation state of CRTC3 with a protein means for binding to phosphorylated CRTC3, thereby determining the level of bound phosphorylated CRTC3.

In one embodiment, the method of screening one or more agents further comprises comparing the level of bound phosphorylated CRTC3 of the composition or the one or more cells with the level of bound phosphorylated CRTC3 of a comparator, wherein: an increase in phosphorylation relative to said comparator indicates that said one or more agents is an enhancer of CRTC3 phosphorylation; and a decrease in phosphorylation relative to said comparator indicates that said one or more agents is an inhibitor of CRTC3 phosphorylation.

In one embodiment, the present invention relates to a method of determining the effect of one or more therapeutics that modulates the level of phosphorylation of CRTC3 in a subject, comprising: administering to the subject the one or more therapeutics; obtaining a sample from the subject; contacting the sample with a protein means for binding to phosphorylated CRTC3; and detecting the level of bound phosphorylated CRTC3 in the sample of the subject as compared to the level of bound phosphorylated CRTC3 of a comparator.

In one embodiment of the method of determining the effect of one or more therapeutics that modulates the level of phosphorylation of CRTC3, an elevated level of bound phosphorylated CRTC3 in the sample of the subject as compared to the level of bound phosphorylated CRTC3 of a comparator indicates one or more selected from the group consisting of: 1) that said one or more therapeutics is efficacious for elevating the level of phosphorylated CTRC3 in a subject and 2) that the concentration of the one or more therapeutics is therapeutically effective.

In one embodiment of the method of determining the effect of one or more therapeutics that modulates the level of phosphorylation of CRTC3, a depressed level of bound phosphorylated CRTC3 in the sample of the subject as compared to the level of bound phosphorylated CRTC3 of a comparator indicates one or more selected from the group consisting of: 1) that said one or more therapeutics is efficacious for depressing the level of phosphorylated CTRC3 in a subject and 2) that the concentration of the one or more therapeutics is therapeutically effective.

In one embodiment, the present invention relates to a method of isolating phosphorylated CRTC3 from a sample, comprising: obtaining the sample; contacting the sample with a protein means for binding to phosphorylated CRTC3, wherein said protein means is conjugated to a substrate; washing the sample to remove any unbound species; and removing the bound phosphorylated CRTC3 in the sample from the protein means that binds phosphorylated CRTC3, thereby generating an isolated composition of phosphorylated CRTC3.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the protein sequence alignment of human (top) and murine (bottom) CRTC3 from amino acid 301 to 420 using the ALIGN sequence alignment tool. The sequences that constitute the antigens for antibody generation are boxed and center around serine residues 329 and 370. In vitro generated peptides with phosphorylated serine residues were used for rabbit immunization. The human CRTC3 amino acid sequence can be found at UniProt ID=Q6UUV7, and the murine CRTC3 amino acid sequence can be found at UniProt ID=Q91X84. The one-letter amino acid code is used.

FIG. 2, comprising FIGS. 2A through 2D, depicts exemplary results demonstrating specific recognition of phosphorylated CRTC3 using monoclonal antibodies. FIG. 2A depicts exemplary results of supernatants from hybridomas #21 (left) or #82 (right), tested for antigenic specificity by western blotting of wild type (lane 1) or S329A mutant CRTC3 (lane 2) protein. FIG. 2B depicts exemplary results supernatants from hybridomas #157 (left) or #170 (right), tested for antigenic specificity by western blotting of S370A mutant (lane 1) or wild type CRTC3 (lane 2) protein as in FIG. 2A. FIG. 2C depicts exemplary results of recombinantly generated mAbs #21 (left) or #82 (right), tested for antigenic specificity by western blotting of wild type (lane 1) or S329A mutant CRTC3 (lane 2) protein as in FIG. 2A. Lanes 3 and 4 show cell lysates of macrophage-differentiated and LPS-stimulated THP1 cells that were either left otherwise untreated (lane 3) or incubated with dasatinib (lane 4) for 1.5h before lysis. FIG. 2D depicts exemplary results of recombinantly generated mAbs #157 (left) or #170 (right), tested for antigenic specificity by western blotting of wild type (lane 1) or S370A mutant CRTC3 (lane 2) protein as in A. Lanes 3 and 4 show THP1 cell lysates treated as in FIG. 2C. A molecular weight marker (M) was run alongside each gel.

### DETAILED DESCRIPTION

The disclosed methods may be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended, and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Aberrant” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antibody-dependent cellular cytotoxicity”, “antibody-dependent cell-mediated cytotoxicity” or “ADCC” refers to the mechanism of inducing cell death that depends upon the interaction of antibody-coated target cells with effector cells possessing lytic activity, such as NK cells, monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) capable of being bound by an antigen binding domain. Antigens may be expressed by genes, synthetized, or purified from biological samples such as a tissue sample, a tumor sample, a cell or a fluid with other biological components, organisms, subunits of proteins/antigens, and killed or inactivated whole cells or lysates.

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as VH, the VL, the VH and the VL, Fab, Fab′, F(ab′)2, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each HC is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes: IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Chimeric antigen receptor” (CAR) as used herein is defined as a cell-surface receptor comprising an extracellular target-binding domain, a transmembrane domain and an intracellular signaling domain, all in a combination that is not naturally found together on a single protein. This includes receptors wherein the extracellular domain and the intracellular signaling domain are not naturally found together on a single receptor protein. CARS are intended primarily for use with lymphocyte such as T cells and NK cells.

“Complement-dependent cytotoxicity” or “CDC”, refers to the mechanism of inducing cell death in which the Fc effector domain of a target-bound protein binds and activates complement component C1q which in turn activates the complement cascade leading to target cell death. Activation of complement may also result in deposition of complement components on the target cell surface that facilitate CDC by binding complement receptors (e.g., CR3) on leukocytes

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g. Lefranc et al. (2003) Dev Comp Immunol 27: 55-77; Honegger and Pluckthun, J Mol Biol (2001) 309:657-70; International ImMunoGeneTics (IMGT) database; Web resources, www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“CREB-regulated transcription coactivator 3” or “CRTC3” refers to a coactivator of cAMP response element-binding (CREB) protein. The amino acid sequences of the various isoforms are retrievable from GenBank accession numbers Q6UUV7.2, XP_024305787.1, XP_024305786.1, XP_011520208.1, XP 005255025.1, NP_073606.3, and NP_001036039.1.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion, T cell activation or T-cell mediated tumor cell killing or binding of a protein to its antigen or receptor, and enhanced binding to a Fey or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

A “Disease” refers to a state of health of an animal wherein the animal cannot maintain homeostasis, and wherein if the disease is not ameliorated then the animal's health continues to deteriorate. In contrast, a “Disorder” in an animal refers to a state of health in which the animal is able to maintain homeostasis, but in which the animal's state of health is less favorable than it would be in the absence of the disorder. Left untreated, a disorder does not necessarily cause a further decrease in the animal's state of health. In some of any one of the above- or below-mentioned embodiments, the disease or disorder comprises Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease or an ulcerative colitis. In some of any one of the above- or below-mentioned embodiments, the disease or disorder comprises an ankylosing spondylitis, an atherosclerosis, an autoimmune diabetes, a primary biliary cholangitis, an inflammatory bowel disease (IBD), a systemic lupus erythematosus, a multiple sclerosis, a psoriasis, an arthritis, Sjogren's syndrome, a transplantation, a graft versus host disease (GVHD), a type I diabetes, a rheumatoid arthritis, a juvenile arthritis, a psoriatic arthritis, Celiac Disease, Crohn's disease or an ulcerative colitis.

“Diagnosis” refers to the determination of the presence of a disease or disorder. In some of any one of the above- or below-mentioned embodiments of the present invention, methods for making a diagnosis are provided which permit determination of the presence of a particular disease or disorder.

“Domain Antibody,” “dAb,” or “dAb fragment” refers to an antibody fragment composed of either VH and the VL domains from a single arm of the antibody.

“Differentiation” refers to a method of decreasing the potency or proliferation of a cell or moving the cell to a more developmentally restricted state.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Enhance,” “promote,” “increase,” “expand” or “improve” refers generally to the ability of a test molecule or agent to mediate a greater response (i.e., downstream effect) when compared to the response mediated by a control or a vehicle. For example, an enhancer of CRTC3 phosphorylation would increase the level of CRTC3 phosphorylation as compared to a control or vehicle. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Expansion” refers to the outcome of cell division and cell death.

“Express” and “expression” refers the to the well-known transcription and translation occurring in cells or in vitro. The expression product, e.g., the protein, is thus expressed by the cell or in vitro and may be an intracellular, extracellular or a transmembrane protein.

“Expression vector” refers to a vector that can be utilized in a biological system or in a reconstituted biological system to direct the translation of a polypeptide encoded by a polynucleotide sequence present in the expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

“dAb” or “dAb fragment” refers to an antibody fragment composed of a VH domain (Ward et al., Nature 341:544 546 (1989)).

“Fab” or “Fab fragment” refers to an antibody fragment composed of VH, CH1, VL and CL domains.

“F(ab′)2” or “F(ab′)2 fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody.

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Genetic modification” refers to the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to the polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are be administered to a subject together in a mixture, concurrently as single agents or sequentially as single agents in any order.

“Inhibit” or “inhibitor” refers generally to the ability of a test molecule or agent to mediate a reduced response (i.e., downstream effect) when compared to the response mediated by a control or a vehicle. For example, an inhibitor of CRTC3 phosphorylation would decrease the level of CRTC3 phosphorylation as compared to a control or vehicle. Inhibition may be a statistically significant difference in the measured response between the test molecule and control (or vehicle), or an decrease in the measured response, such as an decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Minibody” to refers to scFv fragments which are linked via CH3 domains.

“Multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Natural killer cell” and “NK cell” are used interchangeably and synonymously herein. NK cell refers to a differentiated lymphocyte with a CD16+CD56+ and/or CD57+ TCR-phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). “Operatively linked peptide” refers to a peptide in which the functional domains of the peptide are placed with appropriate distance from each other to impart the intended function of each domain.

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Prevent,” “preventing,” “prevention,” or “prophylaxis” of a disease or disorder means preventing a disorder from occurring in a subject.

“Proliferation” refers to an increase in cell division, either symmetric or asymmetric division of cells.

“Promoter” refers to the minimal sequences required to initiate transcription. Promoter may also include enhancers or repressor elements which enhance or suppress transcription, respectively.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Regulatory element” refers to any cis- or trans acting genetic element that controls some aspect of the expression of nucleic acid sequences.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (KD) of about 1×10-7 M or less, for example about 5×10-8 M or less, about 1×10-8 M or less, about 1×10-9 M or less, about 1×10-10 M or less, about 1×10-11 M or less, or about 1×10-12 M or less, typically with a KD that is at least one hundred fold less than its KD for binding to a non-specific antigen (e.g., BSA, casein). In the context of the CRTC3 antigens described here, “specific binding” refers to binding of the proteinaceous molecule to the CRTC3 antigen without detectable binding to a wild-type protein the antigen is a variant of.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“Therapeutically effective amount” or “effective amount” as used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics include, for example, improved wellbeing of the patient, reduction of a tumor burden, arrested or slowed growth of a tumor, and/or absence of metastasis of cancer cells to other locations in the body.

“Transduction” refers to the introduction of a foreign nucleic acid into a cell using a viral vector.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Mutations in the Ig constant regions are referred to as follows: L351Y_F405A_Y407V refers to L351Y, F405A and Y407V mutations in one immunoglobulin constant region. L351Y_F405A_Y407V/T394W refers to L351Y, F405A and Y407V mutations in the first Ig constant region and T394W mutation in the second Ig constant region, which are present in one multimeric protein.

### Phosphorylated-CRTC3 Binding Domains

The present invention describes antibodies raised that specifically bind to at least one phosphorylated but not unphosphorylated serine residue. In some embodiments at least one phosphorylated serine residue is 329, 370, 62 or 162 of human and murine versions of CRTC3. It further describes example methods of their use.

### Compositions of Matter

Antigen Binding Domains that Bind Phosphorylated CRTC3

The disclosure provides antigen binding domains that bind phosphorylated CRTC3, monospecific and multispecific proteins comprising the antigen binding domains that bind phosphorylated CRTC3, polynucleotides encoding the foregoing, vectors, host cells and methods of making and using the foregoing.

In one embodiment, the invention provides an isolated protein comprising an antigen binding domain that binds phosphorylated CREB Regulated Transcription Coactivator 3 (CRTC3), wherein the CRTC3 comprises at least one phosphorylated site. In one embodiment, the phosphorylation site is serine 329. In one embodiment, the phosphorylation site is serine 370. In one embodiment, the phosphorylation site is serine 62. In one embodiment, the phosphorylation site is serine 162.

The disclosure provides an isolated protein comprising an antigen binding domain that binds phosphorylated CRTC3. In one embodiment, the antigen binding domain binds to CRTC3 only when phosphorylated at serine 329. In one embodiment, the antigen binding domain binds to CRTC3 only when phosphorylated at serine 370. In one embodiment, the antigen binding domain binds to CRTC3 only when phosphorylated at serine 62. In one embodiment, the antigen binding domain binds to CRTC3 only when phosphorylated at serine 162.

mAb #157

In one embodiment, the isolated protein comprises an antigen binding domain that binds CRTC3 phosphorylated at serine 370 (pSer370). In one embodiment, the isolated protein comprises an antigen binding domain that binds to the epitope RLFSLpSNPSLST (SEQ ID NO: 145) of CRTC3. In one embodiment, the antigen binding domain comprises a heavy chain complementarity determining region (HCDR) 1 of SEQ ID NOs: 1, 7, 13, 19 or 25.

In one embodiment, the isolated protein comprises an antigen binding domain that binds phosphorylated CRTC3, wherein the antigen binding domain that binds phosphorylated CRTC3 comprises a HCDR2 of SEQ ID NOs: 2, 8, 14, 20 or 26.

In one embodiment, the isolated protein comprises an antigen binding domain that binds phosphorylated CRTC3, wherein the antigen binding domain that binds phosphorylated CRTC3 comprises a HCDR3 of SEQ ID NOs: 3, 9, 15, 21 or 27.

In one embodiment, the isolated protein comprises an antigen binding domain that binds phosphorylated CRTC3, wherein the antigen binding domain that binds phosphorylated CRTC3 comprises a HDCR1 of SEQ ID NOs: 1, 7, 13, 19 or 25; a HCDR2 of SEQ ID NOs: 2, 8, 14, 20 or 26; and a HCDR3 of SEQ ID NOs: 3, 9, 15, 21 or 27.

In one embodiment, the isolated protein comprises an antigen binding domain that binds phosphorylated CRTC3, wherein the antigen binding domain that binds phosphorylated CRTC3 comprises a HCDR1, HCDR2 and HCDR3 of:
SEQ ID NOs: 1, 2, and 3, respectively;
SEQ ID NOs: 7, 8, and 9, respectively;
SEQ ID NOs: 13, 14, and 15, respectively;
SEQ ID NOs: 19, 20, and 21, respectively; or
SEQ ID NOs: 25, 26, and 27, respectively.

In one embodiment, the isolated protein comprises an antigen binding domain that binds phosphorylated CRTC3, wherein the antigen binding domain that binds phosphorylated CRTC3 comprises a light chain complementarity determining region (LCDR) 1 of SEQ ID NOs: 4, 22, or 28.

In one embodiment, the isolated protein comprises an antigen binding domain that binds phosphorylated CRTC3, wherein the antigen binding domain that binds phosphorylated CRTC3 comprises a LCDR2 of SEQ ID NOs: 5, 23, or 29.

In one embodiment, the isolated protein comprises an antigen binding domain that binds phosphorylated CRTC3, wherein the antigen binding domain that binds phosphorylated CRTC3 comprises a LCDR3 of SEQ ID NOs: 6, 24, or 30.

In one embodiment, the isolated protein comprises an antigen binding domain that binds phosphorylated CRTC3, wherein the antigen binding domain that binds phosphorylated CRTC3 comprises a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the isolated protein comprises an antigen binding domain that binds phosphorylated CRTC3, wherein the antigen binding domain that binds phosphorylated CRTC3 comprises a LCDR1, LCDR2 and LCDR3 of:
SEQ ID NOs: 4, 5 and 6, respectively;
SEQ ID NOs: 22, 23 and 24, respectively; or
SEQ ID NOs: 28, 29 and 30, respectively.

In one embodiment, the isolated protein comprises an antigen binding domain that binds phosphorylated CRTC3, wherein the antigen binding domain that binds phosphorylated CRTC3 comprises a HDCR1 of SEQ ID NOs: 1, 7, 13, 19 or 25; a HCDR2 of SEQ ID NOs: 2, 8, 14, 20 or 26; a HCDR3 of SEQ ID NOs: 3, 9, 15, 21 or 27; a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the isolated protein comprises an antigen binding domain that binds phosphorylated CRTC3, wherein the antigen binding domain that binds phosphorylated CRTC3 comprises a HCDR1, HCDR2, HCDR3, LCDR1, LCDR2 and LCDR3 of:
SEQ ID NOs: 1, 2, 3, 4, 5 and 6, respectively;
SEQ ID NOs: 7, 8, 9, 4, 5 and 6, respectively;
SEQ ID NOs: 13, 14, 15, 4, 5 and 6, respectively;
SEQ ID NOs: 19, 20, 21, 22, 23, and 24, respectively; or
SEQ ID NOs: 25, 26, 27, 28, 29, and 30, respectively.
   mAb #170

In one embodiment, the isolated protein comprises an antigen binding domain that binds CRTC3 phosphorylated at serine 370 (pSer370). In one embodiment, the isolated protein comprises an antigen binding domain that binds to the epitope RLFSLpSNPSLST (SEQ ID NO: 145) of CRTC3. In one embodiment, the antigen binding domain comprises a heavy chain complementarity determining region (HCDR) 1 of SEQ ID NOs: 35, 41, 47, 53 or 59.

In one embodiment, the isolated protein comprises an antigen binding domain that binds phosphorylated CRTC3, wherein the antigen binding domain that binds phosphorylated CRTC3 comprises a HCDR2 of SEQ ID NOs: 36, 42, 48, 54 or 60.

In one embodiment, the isolated protein comprises an antigen binding domain that binds phosphorylated CRTC3, wherein the antigen binding domain that binds phosphorylated CRTC3 comprises a HCDR3 of SEQ ID NOs: 37, 43, 49, 55 or 61.

In one embodiment, the isolated protein comprises an antigen binding domain that binds phosphorylated CRTC3, wherein the antigen binding domain that binds phosphorylated CRTC3 comprises a HDCR1 of SEQ ID NOs: 35, 41, 47, 53 or 59; a HCDR2 of SEQ ID NOs: 36, 42, 48, 54 or 60; and a HCDR3 of SEQ ID NOs: 37, 43, 49, 55 or 61.

In one embodiment, the isolated protein comprises an antigen binding domain that binds phosphorylated CRTC3, wherein the antigen binding domain that binds phosphorylated CRTC3 comprises a HCDR1, HCDR2 and HCDR3 of:
SEQ ID NOs: 35, 36, and 37, respectively;
SEQ ID NOs: 41, 42, and 43, respectively;
SEQ ID NOs: 47, 48, and 49, respectively;
SEQ ID NOs: 53, 54, and 55, respectively; or
SEQ ID NOs: 59, 60, and 61, respectively.

In one embodiment, the isolated protein comprises an antigen binding domain that binds phosphorylated CRTC3, wherein the antigen binding domain that binds phosphorylated CRTC3 comprises a light chain complementarity determining region (LCDR) 1 of SEQ ID NOs: 38, 56, or 62.

In one embodiment, the isolated protein comprises an antigen binding domain that binds phosphorylated CRTC3, wherein the antigen binding domain that binds phosphorylated CRTC3 comprises a LCDR2 of SEQ ID NOs: 39, 57, or 63.

In one embodiment, the isolated protein comprises an antigen binding domain that binds phosphorylated CRTC3, wherein the antigen binding domain that binds phosphorylated CRTC3 comprises a LCDR3 of SEQ ID NOs: 40, 58, or 64.

In one embodiment, the isolated protein comprises an antigen binding domain that binds phosphorylated CRTC3, wherein the antigen binding domain that binds phosphorylated CRTC3 comprises a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the isolated protein comprises an antigen binding domain that binds phosphorylated CRTC3, wherein the antigen binding domain that binds phosphorylated CRTC3 comprises a LCDR1, LCDR2 and LCDR3 of:
SEQ ID NOs: 38, 39, and 40, respectively;
SEQ ID NOs: 56, 57, and 58, respectively; or
SEQ ID NOs: 62, 63, and 64, respectively.

In one embodiment, the isolated protein comprises an antigen binding domain that binds phosphorylated CRTC3, wherein the antigen binding domain that binds phosphorylated CRTC3 comprises a HDCR1 of SEQ ID NOs: 35, 41, 47, 53 or 59; a HCDR2 of SEQ ID NOs: 36, 42, 48, 54 or 60; a HCDR3 of SEQ ID NOs: 37, 43, 49, 55 or 61; a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the isolated protein comprises an antigen binding domain that binds phosphorylated CRTC3, wherein the antigen binding domain that binds phosphorylated CRTC3 comprises a HCDR1, HCDR2, HCDR3, LCDR1, LCDR2 and LCDR3 of:
SEQ ID NOs: 35, 36, 37, 38, 39, and 40, respectively;
SEQ ID NOs: 41, 42, 43, 38, 39, and 40, respectively;
SEQ ID NOs: 47, 48, 49, 38, 39, and 40, respectively;
SEQ ID NOs: 53, 54, 55, 56, 57, and 58, respectively; or
SEQ ID NOs: 59, 60, 61, 62, 63, and 64, respectively.
   mAb #21

In one embodiment, the isolated protein comprises an antigen binding domain that binds CRTC3 phosphorylated at serine 329 (pSer329). In one embodiment, the isolated protein comprises an antigen binding domain that binds to the epitope GLQSSRpSNPSIQ (SEQ ID NO: 143) of CRTC3. In one embodiment, the antigen binding domain comprises a heavy chain complementarity determining region (HCDR) 1 of SEQ ID NOs: 69, 75, 81, 87, or 93.

In one embodiment, the isolated protein comprises an antigen binding domain that binds phosphorylated CRTC3, wherein the antigen binding domain that binds phosphorylated CRTC3 comprises a HCDR2 of SEQ ID NOs: 70, 76, 82, 88, or 94.

In one embodiment, the isolated protein comprises an antigen binding domain that binds phosphorylated CRTC3, wherein the antigen binding domain that binds phosphorylated CRTC3 comprises a HCDR3 of SEQ ID NOs: 71, 77, 83, 89, or 95.

In one embodiment, the isolated protein comprises an antigen binding domain that binds phosphorylated CRTC3, wherein the antigen binding domain that binds phosphorylated CRTC3 comprises a HDCR1 of SEQ ID NOs: 69, 75, 81, 87, or 93; a HCDR2 of SEQ ID NOs: 70, 76, 82, 88, or 94; and a HCDR3 of SEQ ID NOs: 71, 77, 83, 89, or 95.

In one embodiment, the isolated protein comprises an antigen binding domain that binds phosphorylated CRTC3, wherein the antigen binding domain that binds phosphorylated CRTC3 comprises a HCDR1, HCDR2 and HCDR3 of:
SEQ ID NOs: 69, 70, and 71, respectively;
SEQ ID NOs: 75, 76, and 77, respectively;
SEQ ID NOs: 81, 82, and 83, respectively;
SEQ ID NOs: 87, 88, and 89, respectively; or
SEQ ID NOs: 93, 94, and 95, respectively.

In one embodiment, the isolated protein comprises an antigen binding domain that binds phosphorylated CRTC3, wherein the antigen binding domain that binds phosphorylated CRTC3 comprises a light chain complementarity determining region (LCDR) 1 of SEQ ID NOs: 72, 90, or 96.

In one embodiment, the isolated protein comprises an antigen binding domain that binds phosphorylated CRTC3, wherein the antigen binding domain that binds phosphorylated CRTC3 comprises a LCDR2 of SEQ ID NOs: 73, 91, or 97.

In one embodiment, the isolated protein comprises an antigen binding domain that binds phosphorylated CRTC3, wherein the antigen binding domain that binds phosphorylated CRTC3 comprises a LCDR3 of SEQ ID NOs: 74, 92, or 98.

In one embodiment, the isolated protein comprises an antigen binding domain that binds phosphorylated CRTC3, wherein the antigen binding domain that binds phosphorylated CRTC3 comprises a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the isolated protein comprises an antigen binding domain that binds phosphorylated CRTC3, wherein the antigen binding domain that binds phosphorylated CRTC3 comprises a LCDR1, LCDR2 and LCDR3 of:
SEQ ID NOs: 72, 73, and 74, respectively;
SEQ ID NOs: 90, 91, and 92, respectively; or
SEQ ID NOs: 96, 97, and 98, respectively.

In one embodiment, the isolated protein comprises an antigen binding domain that binds phosphorylated CRTC3, wherein the antigen binding domain that binds phosphorylated CRTC3 comprises a HDCR1 of SEQ ID NOs: 69, 75, 81, 87, or 93; a HCDR2 of SEQ ID NOs: 70, 76, 82, 88, or 94; a HCDR3 of SEQ ID NOs: 71, 77, 83, 89, or 95; a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the isolated protein comprises an antigen binding domain that binds phosphorylated CRTC3, wherein the antigen binding domain that binds phosphorylated CRTC3 comprises a HCDR1, HCDR2, HCDR3, LCDR1, LCDR2 and LCDR3 of:
SEQ ID NOs: 69, 70, 71, 72, 73, and 74, respectively;
SEQ ID NOs: 75, 76, 77, 72, 73, and 74, respectively;
SEQ ID NOs: 81, 82, 83, 72, 73, and 74, respectively;
SEQ ID NOs: 87, 88, 89, 90, 91, and 92, respectively; or
SEQ ID NOs: 93, 94, 95, 96, 97, and 98 respectively.
   mAb #82

In one embodiment, the isolated protein comprises an antigen binding domain that binds CRTC3 phosphorylated at serine 329 (pSer329). In one embodiment, the isolated protein comprises an antigen binding domain that binds to the epitope GLQSSRpSNPSIQ (SEQ ID NO: 143) of CRTC3. In one embodiment, the antigen binding domain comprises a heavy chain complementarity determining region (HCDR) 1 of SEQ ID NOs: 103, 109, 115, 121, or 127.

In one embodiment, the isolated protein comprises an antigen binding domain that binds phosphorylated CRTC3, wherein the antigen binding domain that binds phosphorylated CRTC3 comprises a HCDR2 of SEQ ID NOs: 104, 110, 116, 122, or 128.

In one embodiment, the isolated protein comprises an antigen binding domain that binds phosphorylated CRTC3, wherein the antigen binding domain that binds phosphorylated CRTC3 comprises a HCDR3 of SEQ ID NOs: 105, 111, 117, 123, or 129.

In one embodiment, the isolated protein comprises an antigen binding domain that binds phosphorylated CRTC3, wherein the antigen binding domain that binds phosphorylated CRTC3 comprises a HDCR1 of SEQ ID NOs: 103, 109, 115, 121, or 127; a HCDR2 of SEQ ID NOs: 104, 110, 116, 122, or 128; and a HCDR3 of SEQ ID NOs: 105, 111, 117, 123, or 129.

In one embodiment, the isolated protein comprises an antigen binding domain that binds phosphorylated CRTC3, wherein the antigen binding domain that binds phosphorylated CRTC3 comprises a HCDR1, HCDR2 and HCDR3 of:
SEQ ID NOs: 103, 104, and 105, respectively;
SEQ ID NOs: 109, 110, and 111, respectively;
SEQ ID NOs: 115, 116, and 117, respectively;
SEQ ID NOs: 121, 122, and 123, respectively; or
SEQ ID NOs: 127, 128, and 129, respectively.

In one embodiment, the isolated protein comprises an antigen binding domain that binds phosphorylated CRTC3, wherein the antigen binding domain that binds phosphorylated CRTC3 comprises a light chain complementarity determining region (LCDR) 1 of SEQ ID NOs: 106, 124, or 130.

In one embodiment, the isolated protein comprises an antigen binding domain that binds phosphorylated CRTC3, wherein the antigen binding domain that binds phosphorylated CRTC3 comprises a LCDR2 of SEQ ID NOs: 107, 125, or 131.

In one embodiment, the isolated protein comprises an antigen binding domain that binds phosphorylated CRTC3, wherein the antigen binding domain that binds phosphorylated CRTC3 comprises a LCDR3 of SEQ ID NOs: 108, 126, or 132.

In one embodiment, the isolated protein comprises an antigen binding domain that binds phosphorylated CRTC3, wherein the antigen binding domain that binds phosphorylated CRTC3 comprises a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the isolated protein comprises an antigen binding domain that binds phosphorylated CRTC3, wherein the antigen binding domain that binds phosphorylated CRTC3 comprises a LCDR1, LCDR2 and LCDR3 of:
SEQ ID NOs: 106, 107, and 108, respectively;
SEQ ID NOs: 124, 125, and 126, respectively; or
SEQ ID NOs: 130, 131, and 132, respectively.

In one embodiment, the isolated protein comprises an antigen binding domain that binds phosphorylated CRTC3, wherein the antigen binding domain that binds phosphorylated CRTC3 comprises a HDCR1 of SEQ ID NOs: 103, 109, 115, 121, or 127; a HCDR2 of SEQ ID NOs: 104, 110, 116, 122, or 128; a HCDR3 of SEQ ID NOs: 105, 111, 117, 123, or 129; a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the isolated protein comprises an antigen binding domain that binds phosphorylated CRTC3, wherein the antigen binding domain that binds phosphorylated CRTC3 comprises a HCDR1, HCDR2, HCDR3, LCDR1, LCDR2 and LCDR3 of:
SEQ ID NOs: 103, 104, 105, 106, 107, and 108, respectively;
SEQ ID NOs: 109, 110, 111, 106, 107, and 108, respectively;
SEQ ID NOs: 115, 116, 117, 106, 107, and 108, respectively;
SEQ ID NOs: 121, 122, 123, 124, 125, and 126, respectively; or
SEQ ID NOs: 127, 128, 129, 130, 131, and 132 respectively.

In one embodiment, the isolated protein comprises an antigen binding domain that binds phosphorylated CRTC3, wherein the antigen binding domain that binds phosphorylated CRTC3 comprises a VH of SEQ ID NOs: 31, 65, 99, or 133.

In one embodiment, the isolated protein comprises an antigen binding domain that binds phosphorylated CRTC3, wherein the antigen binding domain that binds phosphorylated CRTC3 comprises a VH encoded by a nucleic acid sequence of SEQ ID NOs: 179, 181, 183, or 185.

In one embodiment, the isolated protein comprises an antigen binding domain that binds phosphorylated CRTC3, wherein the antigen binding domain that binds phosphorylated CRTC3 comprises a VL of SEQ ID NOs: 32, 66, 100, or 134.

In one embodiment, the isolated protein comprises an antigen binding domain that binds phosphorylated CRTC3, wherein the antigen binding domain that binds phosphorylated CRTC3 comprises a VL encoded by a nucleic acid sequence of SEQ ID NOs: 180, 182, 184, or 186.

In one embodiment, the isolated protein comprises an antigen binding domain that binds phosphorylated CRTC3, wherein the antigen binding domain that binds phosphorylated CRTC3 comprises a VH of SEQ ID NOs: 31, 65, 99, or 133; and the VL of SEQ ID NOs: 32, 66, 100, or 134.

In one embodiment, the isolated protein comprises an antigen binding domain that binds phosphorylated CRTC3, wherein the antigen binding domain that binds phosphorylated CRTC3 comprises a VH encoded by a nucleic acid sequence of SEQ ID NOs: 179, 181, 183, or 185; and the VL encoded by a nucleic acid sequence SEQ ID NOs: 180, 182, 184, or 186.

In one embodiment, the isolated protein comprises an antigen binding domain that binds phosphorylated CRTC3, wherein the antigen binding domain that binds phosphorylated CRTC3 comprises a VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

In one embodiment, the isolated protein comprises an antigen binding domain that binds phosphorylated CRTC3, wherein the antigen binding domain that binds phosphorylated CRTC3 comprises a VH and VL encoded by a nucleic acid sequence of:
SEQ ID NOs: 179 and 180, respectively;
SEQ ID NOs: 181 and 182, respectively;
SEQ ID NOs: 183 and 184, respectively; or
SEQ ID NOs: 185 and 186, respectively.

In one embodiment, the isolated protein comprises an antigen binding domain that binds phosphorylated CRTC3, wherein the antigen binding domain that binds phosphorylated CRTC3 comprises a heavy chain (HC) of SEQ ID NOs: 33, 67, 101, or 135.

In one embodiment, the isolated protein comprises an antigen binding domain that binds phosphorylated CRTC3, wherein the antigen binding domain that binds phosphorylated CRTC3 comprises a light chain (LC) of SEQ ID NOs: 34, 68, 102, or 136.

In one embodiment, the isolated protein comprises an antigen binding domain that binds phosphorylated CRTC3, wherein the antigen binding domain that binds phosphorylated CRTC3 comprises a HC of SEQ ID NOs: 33, 67, 101, or 135; and a LC of SEQ ID NOs: 34, 68, 102, or 136.

In one embodiment, the isolated protein comprises an antigen binding domain that binds phosphorylated CRTC3, wherein the antigen binding domain that binds phosphorylated CRTC3 comprises the HC and LC of:
SEQ ID NOs: 33 and 34, respectively;
SEQ ID NOs: 67 and 68, respectively;
SEQ ID NOs: 101 and 102, respectively; or
SEQ ID NOs: 135 and 136, respectively.

In some of any one of the above- or below-mentioned embodiments, the isolated protein comprising an antigen binding domain that binds phosphorylated CRTC3 is a scFv.

In some of any one of the above- or below-mentioned embodiments, the isolated protein comprising an antigen binding domain that binds phosphorylated CRTC3 is a (scFv)2.

In some of any one of the above- or below-mentioned embodiments, the isolated protein comprising an antigen binding domain that binds phosphorylated CRTC3 is a Fv.

In some of any one of the above- or below-mentioned embodiments, the isolated protein comprising an antigen binding domain that binds phosphorylated CRTC3 is a Fab.

In some of any one of the above- or below-mentioned embodiments, the isolated protein comprising an antigen binding domain that binds phosphorylated CRTC3 is a F(ab′)2.

In some of any one of the above- or below-mentioned embodiments, the isolated protein comprising an antigen binding domain that binds phosphorylated CRTC3 is a Fd.

In some of any one of the above- or below-mentioned embodiments, the isolated protein comprising an antigen binding domain that binds phosphorylated CRTC3 is a dAb.

In some of any one of the above- or below-mentioned embodiments, the isolated protein comprising an antigen binding domain that binds phosphorylated CRTC3 is a VHH.

### Multispecific Proteins

In some of any one of the above- or below-mentioned embodiments, the disclosure provides a multispecific protein (e.g., a multispecific antibody) comprising an antigen binding domain that binds phosphorylated CRTC3. For example, in one embodiment, the antigen binding domains that bind phosphorylated CRTC3 can be incorporated into the Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Phosphorylated CRTC3 Binding scFvs

Any of the VH and the VL domains identified herein that bind phosphorylated CRTC3 may be engineered into scFv format in either VH-linker-VL or VL-linker-VH orientation. Any of the VH and the VL domains identified herein may also be used to generate sc(Fv)2 structures, such as VH-linker-VL-linker-VL-linker-VH, VH-linker-VL-linker-VH-linker-VL, VH-linker-VH-linker-VL-linker-VL, VL-linker-VH-linker-VH-linker-VL, VL-linker-VH-linker-VL-linker-VH or VL-linker-VL-linker-VH-linker-VH.

VH and the VL domains identified herein may be incorporated into a scFv format and the binding and thermostability of the resulting scFv to phosphorylated CRTC3 may be assessed using known methods. Binding may be assessed using ProteOn XPR36, Biacore 3000 or KinExA instrumentation, ELISA or competitive binding assays known to those skilled in the art. Binding may be evaluated using purified scFvs or E. coli supernatants or lysed cells containing the expressed scFv. The measured affinity of a test scFv to CRTC3 may vary if measured under different conditions (e.g., osmolarity, pH). Thus, measurements of affinity and other binding parameters (e.g., KD, Kon, Koff) are typically made with standardized conditions and standardized buffers. Thermostability may be evaluated by heating the test scFv at elevated temperatures, such as at 50° C., 55° C. or 60° C. for a period of time, such as 5 minutes (min), 10 min, 15 min, 20 min, 25 min or 30 min and measuring binding of the test scFv to phosphorylated CRTC3. The scFvs retaining comparable binding to phosphorylated CRTC3 when compared to a non-heated scFv sample are referred to as being thermostable.

In recombinant expression systems, the linker is a peptide linker and may include any naturally occurring amino acid. Exemplary amino acids that may be included into the linker are Gly, Ser, Pro, Thr, Glu, Lys, Arg, Ile, Leu, His and The. The linker should have a length that is adequate to link the VH and the VL in such a way that they form the correct conformation relative to one another so that they retain the desired activity, such as binding to phosphorylated CRTC3.

The linker may be about 5-50 amino acids long. In some of any one of the above- or below-mentioned embodiments, the linker is about 10-40 amino acids long. In some embodiments, the linker is about 10-35 amino acids long. In some of any one of the above- or below-mentioned embodiments, the linker is about 10-30 amino acids long. In some of any one of the above- or below-mentioned embodiments, the linker is about 10-25 amino acids long. In some of any one of the above- or below-mentioned embodiments, the linker is about 10-20 amino acids long. In some of any one of the above- or below-mentioned embodiments, the linker is about 15-20 amino acids long. In some of any one of the above- or below-mentioned embodiments, the linker is 6 amino acids long. In some of any one of the above- or below-mentioned embodiments, the linker is 7 amino acids long. In some of any one of the above- or below-mentioned embodiments, the linker is 8 amino acids long. In some of any one of the above- or below-mentioned embodiments, the linker is 9 amino acids long. In some of any one of the above- or below-mentioned embodiments, the linker is 10 amino acids long. In some of any one of the above- or below-mentioned embodiments, the linker is 11 amino acids long. In some of any one of the above- or below-mentioned embodiments, the linker is 12 amino acids long. In some of any one of the above- or below-mentioned embodiments, the linker is 13 amino acids long. In some of any one of the above- or below-mentioned embodiments, the linker is 14 amino acids long. In some of any one of the above- or below-mentioned embodiments, the linker is 15 amino acids long. In some of any one of the above- or below-mentioned embodiments, the linker is 16 amino acids long. In some of any one of the above- or below-mentioned embodiments, the linker is 17 amino acids long. In some of any one of the above- or below-mentioned embodiments, the linker is 18 amino acids long. In some of any one of the above- or below-mentioned embodiments, the linker is 19 amino acids long. In some of any one of the above- or below-mentioned embodiments, the linker is 20 amino acids long. In some of any one of the above- or below-mentioned embodiments, the linker is 21 amino acids long. In some of any one of the above- or below-mentioned embodiments, the linker is 22 amino acids long. In some of any one of the above- or below-mentioned embodiments, the linker is 23 amino acids long. In some of any one of the above- or below-mentioned embodiments, the linker is 24 amino acids long. In some of any one of the above- or below-mentioned embodiments, the linker is 25 amino acids long. In some of any one of the above- or below-mentioned embodiments, the linker is 26 amino acids long. In some of any one of the above- or below-mentioned embodiments, the linker is 27 amino acids long. In some of any one of the above- or below-mentioned embodiments, the linker is 28 amino acids long. In some of any one of the above- or below-mentioned embodiments, the linker is 29 amino acids long. In some of any one of the above- or below-mentioned embodiments, the linker is 30 amino acids long. In some of any one of the above- or below-mentioned embodiments, the linker is 31 amino acids long. In some of any one of the above- or below-mentioned embodiments, the linker is 32 amino acids long. In some of any one of the above- or below-mentioned embodiments, the linker is 33 amino acids long. In some of any one of the above- or below-mentioned embodiments, the linker is 34 amino acids long. In some of any one of the above- or below-mentioned embodiments, the linker is 35 amino acids long. In some of any one of the above- or below-mentioned embodiments, the linker is 36 amino acids long. In some of any one of the above- or below-mentioned embodiments, the linker is 37 amino acids long. In some of any one of the above- or below-mentioned embodiments, the linker is 38 amino acids long. In some of any one of the above- or below-mentioned embodiments, the linker is 39 amino acids long. In some of any one of the above- or below-mentioned embodiments, the linker is 40 amino acids long. Exemplary linkers that may be used are Gly rich linkers, Gly and Ser containing linkers, Gly and Ala containing linkers, Ala and Ser containing linkers, and other flexible linkers.

Other linker sequences may include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Additional linkers are described for example in Int. Pat. Publ. No. WO2019/060695.

In some of any one of the above- or below-mentioned embodiments, the scFv comprises, from the N- to C-terminus, a VH, a first linker (L1) and a VL (VH-L1-VL). In some of any one of the above- or below-mentioned embodiments, the scFv comprises, from the N- to C-terminus, the VL, the L1 and the VH (VL-L1-VH).

Other Antigen Binding Domains that Bind Phosphorylated CRTC3

Any of the VH and the VL domains identified herein that bind phosphorylated CRTC3 may also be engineered into Fab, F(ab′)2, Fd or Fv format and their binding to phosphorylated CRTC3 may be assessed using the assays described herein.

Homologous Antigen Binding Domains and Antigen Binding Domains with Conservative Substitutions

Variants of the antigen binding domains that bind phosphorylated CRTC3 are within the scope of the disclosure. For example, variants may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29 or more amino acid substitutions in the antigen binding domain that bind phosphorylated CRTC3 as long as they retain or have improved functional properties when compared to the parent antigen binding domains. In some of any one of the above- or below-mentioned embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind phosphorylated CRTC3 of the disclosure. In some of any one of the above- or below-mentioned embodiments, the variation is in the framework regions. In some of any one of the above- or below-mentioned embodiments, variants are generated by conservative substitutions.

Also provided are antigen binding domains that bind phosphorylated CRTC3 comprising the VH and the VL which are at least 80% identical to the VH and VL of
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

In some of any one of the above- or below-mentioned embodiments, the identity is at least 85%. In some of any one of the above- or below-mentioned embodiments, the identity is at least 90%. In some of any one of the above- or below-mentioned embodiments, the identity is at least 91%. In some of any one of the above- or below-mentioned embodiments, the identity is at least 91%. In some of any one of the above- or below-mentioned embodiments, the identity is at least 92%. In some of any one of the above- or below-mentioned embodiments, the identity is at least 93%. In some of any one of the above- or below-mentioned embodiments, the identity is at least 94%. In some of any one of the above- or below-mentioned embodiments, the identity is at least 95%. In some of any one of the above- or below-mentioned embodiments, the identity is at least 96%. In some of any one of the above- or below-mentioned embodiments, the identity is at least 97%. In some of any one of the above- or below-mentioned embodiments, the identity is at least 98%. In some of any one of the above- or below-mentioned embodiments, the identity is at least 99%.

The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at www_gcg_com), using either a Blossum 62 matrix or a PAM250 matrix, and a gap weight of 16, 14, 12, 10, 8, 6, or 4 and a length weight of 1, 2, 3, 4, 5, or 6.

In some of any one of the above- or below-mentioned embodiments, variant antigen binding domains that bind phosphorylated CRTC3 comprise one or two conservative substitutions in any of the CDR regions, while retaining desired functional properties of the parent antigen binding fragments that bind phosphorylated CRTC3.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

Methods of Generating Antigen Binding Fragment that Bind Phosphorylated CRTC3

Antigen binding domains that bind phosphorylated CRTC3 provided in the disclosure may be generated using various technologies. For example, the hybridoma method of Kohler and Milstein may be used to identify VH/VL pairs that bind phosphorylated CRTC3. In the hybridoma method, a mouse or other host animal, such as a hamster, rat or chicken is immunized with phosphorylated CRTC3, followed by fusion of spleen cells from immunized animals with myeloma cells using standard methods to form hybridoma cells. Colonies arising from single immortalized hybridoma cells may be screened for production of the antibodies containing the antigen binding domains that bind phosphorylated CRTC3 with desired properties, such as specificity of binding, cross-reactivity or lack thereof, affinity for the antigen, and any desired functionality.

Antigen binding domains that bind phosphorylated CRTC3 generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1990/007861 and WO1992/22653, or by introducing variation at any of the CDRs for example to improve affinity of the antigen binding domain.

Transgenic animals, such as mice, rat or chicken carrying human immunoglobulin (Ig) loci in their genome may be used to generate antigen binding fragments that bind phosphorylated CRTC3, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (www_regeneron_com), Harbour Antibodies (www_harbourantibodies_com), Open Monoclonal Technology, Inc. (OMT) (www_omtinc net), KyMab (www kymab_com), Trianni (www.trianni_com) and Ablexis (www_ablexis_com) may be engaged to provide human antibodies directed against a selected antigen using technologies as described above.

Antigen binding domains that bind phosphorylated CRTC3 may be selected from a phage display library, where the phage is engineered to express human immunoglobulins or portions thereof such as Fabs, single chain antibodies (scFv), or unpaired or paired antibody variable regions. The antigen binding domains that bind CRTC3 may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno CRTC3 and the obtained positive clones may be further characterized, the Fabs isolated from the clone lysates, and converted to scFvs or other configurations of antigen binding fragments.

Preparation of immunogenic antigens and expression and production of antigen binding domains of the disclosure may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

### Conjugation to Half-Life Extending Moieties

The antigen binding domains that bind phosphorylated CRTC3 of the disclosure may be conjugated to a half-life extending moiety. Exemplary half-life extending moieties are albumin, albumin variants, albumin-binding proteins and/or domains, transferrin and fragments and analogues thereof, immunoglobulins (Ig) or fragments thereof, such as Fc regions. Amino acid sequences of the aforementioned half-life extending moieties are known. Ig or fragments thereof include all isotypes, i.e., IgG1, IgG2, IgG3, IgG4, IgM, IgA and IgE.

Additional half-life extending moieties that may be conjugated to the antigen binding domains that bind phosphorylated CRTC3 of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antigen binding domains that bind phosphorylated CRTC3 of the disclosure and may be generated by standard cloning and expression techniques. Alternatively, well known chemical coupling methods may be used to attach the moieties to recombinantly produced antigen binding domains that bind phosphorylated CRTC3 of the disclosure.

A pegyl moiety may for example be conjugated to the antigen binding domain that bind phosphorylated CRTC3 of the disclosure by incorporating a cysteine residue to the C-terminus of the antigen binding domain that bind phosphorylated CRTC3 of the disclosure, or engineering cysteines into residue positions that face away from the phosphorylated CRTC3-binding site and attaching a pegyl group to the cysteine using well known methods.

In some of any one of the above- or below-mentioned embodiments, the antigen binding fragment that binds phosphorylated CRTC3 is conjugated to a half-life extending moiety.

In some of any one of the above- or below-mentioned embodiments, the half-life extending moiety is an immunoglobulin (Ig), a fragment of the Ig, an Ig constant region, a fragment of the Ig constant region, a Fc region, transferrin, albumin, an albumin binding domain or polyethylene glycol. In some of any one of the above- or below-mentioned embodiments, the half-life extending moiety is an Ig constant region.

In some of any one of the above- or below-mentioned embodiments, the half-life extending moiety is the Ig.

In some of any one of the above- or below-mentioned embodiments, the half-life extending moiety is the fragment of the Ig.

In some of any one of the above- or below-mentioned embodiments, the half-life extending moiety is the Ig constant region.

In some of any one of the above- or below-mentioned embodiments, the half-life extending moiety is the fragment of the Ig constant region.

In some of any one of the above- or below-mentioned embodiments, the half-life extending moiety is the Fc region.

In some of any one of the above- or below-mentioned embodiments, the half-life extending moiety is albumin.

In some of any one of the above- or below-mentioned embodiments, the half-life extending moiety is the albumin binding domain.

In some of any one of the above- or below-mentioned embodiments, the half-life extending moiety is transferrin.

In some of any one of the above- or below-mentioned embodiments, the half-life extending moiety is polyethylene glycol.

The antigen binding domains that bind CRTC3 conjugated to a half-life extending moiety may be evaluated for their pharmacokinetic properties utilizing known in vivo models. Isotypes, allotypes and Fc engineering

The Ig constant region or the fragment of the Ig constant region, such as the Fc region present in the proteins of the disclosure may be of any allotype or isotype.

In some of any one of the above- or below-mentioned embodiments, the Ig constant region or the fragment of the Ig constant region is an IgG1 isotype.

In some of any one of the above- or below-mentioned embodiments, the Ig constant region or the fragment of the Ig constant region is an IgG2 isotype.

In some of any one of the above- or below-mentioned embodiments, the Ig constant region or the fragment of the Ig constant region is an IgG3 isotype.

In some of any one of the above- or below-mentioned embodiments, the Ig constant region or the fragment of the Ig constant region is an IgG4 isotype.

The Ig constant region or the fragment of the Ig constant region may be of any allotype. It is expected that allotype has no influence on properties of the Ig constant region, such as binding or Fc-mediated effector functions. Immunogenicity of therapeutic proteins comprising Ig constant regions of fragments thereof is associated with increased risk of infusion reactions and decreased duration of therapeutic response (Baert et al., (2003) N. Engl. J. Med. 348:602-08). The extent to which therapeutic proteins comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody. Table 1 shows select IgG1, IgG2 and IgG4 allotypes.

TABLE 1

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| G1m(17) | | | | | K | E | M | A |
| G1m(17, 1) | | | | | K | D | L | A |

C-terminal lysine (CTL) may be removed from the Ig constant region by endogenous circulating carboxypeptidases in the bloodstream (Cai et al., (2011) Biotechnol. Bioeng. 108:404-412). During manufacturing, CTL removal may be controlled to less than the maximum level by control of concentration of extracellular Zn2+, EDTA or EDTA-Fe3+ as described in U.S. Patent Publ. No. US20140273092. CTL content of proteins may be measured using known methods.

In some of any one of the above- or below-mentioned embodiments, the antigen binding fragment that binds phosphorylated CRTC3 conjugated to the Ig constant region has a C-terminal lysine content from about 10% to about 90%. In some of any one of the above- or below-mentioned embodiments, the C-terminal lysine content is from about 20% to about 80%. In some of any one of the above- or below-mentioned embodiments, the C-terminal lysine content is from about 40% to about 70%. In some of any one of the above- or below-mentioned embodiments, the C-terminal lysine content is from about 55% to about 70%. In some of any one of the above- or below-mentioned embodiments, the C-terminal lysine content is about 60%.

Fc region mutations may be made to the antigen binding domains that bind phosphorylated CRTC3 conjugated to the Ig constant region or to the fragment of the Ig constant region to modulate their effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some of any one of the above- or below-mentioned embodiments, the antigen binding domain that binds phosphorylated CRTC3 conjugated to the Ig constant region or the fragment of the Ig constant region comprises at least one mutation in the Ig constant region or in the fragment of the Ig constant region.

In some of any one of the above- or below-mentioned embodiments, the at least one mutation is in the Fc region.

In some of any one of the above- or below-mentioned embodiments, the antigen binding domain that binds phosphorylated CRTC3 conjugated to the Ig constant region or to the fragment of the Ig constant region comprises at least one, two, three, four, five, six, seven, eight, nine, ten, eleven, twelve, thirteen, fourteen or fifteen mutations in the Fc region.

In some of any one of the above- or below-mentioned embodiments, the antigen binding domain that binds phosphorylated CRTC3 conjugated to the Ig constant region or to the fragment of the Ig constant region comprises at least one mutation in the Fc region that modulates binding of the antibody to FcRn.

Fc positions that may be mutated to modulate half-life (e.g. binding to FcRn) include positions 250, 252, 253, 254, 256, 257, 307, 376, 380, 428, 434 and 435. Exemplary mutations that may be made singularly or in combination are mutations T250Q, M252Y, I253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A. Exemplary singular or combination mutations that may be made to reduce the half-life are mutations H435A, P257I/N434H, D376V/N434H, M252Y/S254T/T256E/H433K/N434F, T308P/N434A and H435R.

In some of any one of the above- or below-mentioned embodiments, the antigen binding domain that binds CRTC3 conjugated to the Ig constant region or to the fragment of the Ig constant region comprises M252Y/S254T/T256E mutation.

In some of any one of the above- or below-mentioned embodiments, the antigen binding domain that binds CRTC3 conjugated to the Ig constant region or to the fragment of the Ig constant region comprises at least one mutation in the Fc region that reduces binding of the protein to an activating Fey receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of the protein to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in proteins with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

An exemplary mutation that results in proteins with reduced CDC is a K322A mutation.

Well-known S228P mutation may be made in IgG4 to enhance IgG4 stability.

In some of any one of the above- or below-mentioned embodiments, the antigen binding domain that binds CRTC3 conjugated to the Ig constant region or to the fragment of the Ig constant region comprises at least one mutation selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, K322, A330S and P331S.

In some of any one of the above- or below-mentioned embodiments, the antigen binding domain that binds CRTC3 conjugated to the Ig constant region or to the fragment of the Ig constant region comprises L234A/L235A/D265S mutation.

In some of any one of the above- or below-mentioned embodiments, the antigen binding domain that binds CRTC3 conjugated to the Ig constant region or to the fragment of the Ig constant region comprises L234A/L235A mutation.

In some of any one of the above- or below-mentioned embodiments, the antigen binding domain that binds CRTC3 conjugated to the Ig constant region or to the fragment of the Ig constant region comprises at least one mutation in the Fc region that enhances binding of the protein to an Fcγ receptor (FcγR) and/or enhances Fc effector functions such as C1q binding, complement dependent cytotoxicity (CDC), antibody-dependent cell-mediated cytotoxicity (ADCC) and/or phagocytosis (ADCP).

Fc positions that may be mutated to increase binding of the protein to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in proteins with increased ADCC or ADCP are a S239D/I332E, S298A/E333A/K334A, F243L/R292P/Y300L, F243L/R292P/Y300L/P396L, F243L/R292P/Y300L/V305I/P396L and G236A/S239D/I332E.

Fc positions that may be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430S, E430F and E430T. Exemplary combination mutations that result in proteins with increased CDC are K326A/E333A, K326W/E333A, H268F/S324T, S267E/H268F, S267E/S324T and S267E/H268F/S324T.

The specific mutations described herein are mutations when compared to the IgG1, IgG2 and IgG4 wild-type amino acid sequences of SEQ ID NOs:179, 180, and 181, respectively.

-wild-type IgG1
SEQ ID NO: 179
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
-wild-type IgG2
SEQ ID NO: 180
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
-wild-type IgG4
SEQ ID NO: 181
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

Proteins Comprising the Antigen Binding Domains that Bind Phosphorylated CRTC3 of the Disclosure

The antigen binding domains that bind phosphorylated CRTC3 of the disclosure may be engineered into monospecific or multispecific proteins of various designs using standard methods. The disclosure also provides a monospecific protein comprising the antigen binding domain that binds phosphorylated CRTC3 of the disclosure. In some of any one of the above- or below-mentioned embodiments, the monospecific protein is an antibody.

While not being limited by this approach, in general when constructing antibodies as multi-specific antibodies, the binding domain modules to each target (first, second, third etc) are optionally built from scFv, Fab, Fab′, F(ab′)2, Fv, variable domain (e.g. VH or VL), diabody, minibody or full length antibodies. For example, each said binding domain or module is created in one or more of the following non-limiting formats wherein binding domains comprising variable domains, and/or full length antibodies, and/or antibody fragments, are operatively linked in series to generate multi-specific antibodies.

In one embodiment there is provided a multi-specific antibody comprising at least one first antibody-derived binding domain targeting phosphorylated CRTC3 and which is operatively linked to at least one second antibody binding domain targeting a second epitope. Optionally, the binding domains comprise at least one or more VH and cognate VL binding domain, or one or more VH-CH1-CH2-CH2 and cognate VL-CL binding domain, or one or more antibody fragment binding domains.

The disclosure also provides a multispecific protein comprising the antigen binding domain that binds phosphorylated CRTC3 of the disclosure.

In some of any one of the above- or below-mentioned embodiments, the multispecific protein is bispecific.

In some of any one of the above- or below-mentioned embodiments, the multispecific protein is trispecific.

In some of any one of the above- or below-mentioned embodiments, the multispecific protein is tetraspecific.

In some of any one of the above- or below-mentioned embodiments, the multispecific protein is monovalent for binding to phosphorylated CRTC3.

In some of any one of the above- or below-mentioned embodiments, the multispecific protein is bivalent for binding to phosphorylated CRTC3.

The disclosure also provides an isolated multispecific protein comprising a first antigen binding domain that binds phosphorylated CRTC3 and a second antigen binding domain that binds a second antigen. In some of any one of the above- or below-mentioned embodiments, the first antigen binding domain that binds CRTC3 and/or the second antigen binding domain that binds the second antigen comprise a scFv, a (scFv)2, a Fv, a Fab, a F(ab′)2, a Fd, a dAb or a VHH.

In some of any one of the above- or below-mentioned embodiments, the first antigen binding domain that binds phosphorylated CRTC3 and/or the second antigen binding domain that binds the second antigen comprise the Fab.

In some of any one of the above- or below-mentioned embodiments, the first antigen binding domain that binds phosphorylated CRTC3 and/or the second antigen binding domain that binds the second antigen comprise the F(ab′)2.

In some of any one of the above- or below-mentioned embodiments, the first antigen binding domain that binds phosphorylated CRTC3 and/or the second antigen binding domain that binds the second antigen comprise the VHH.

In some of any one of the above- or below-mentioned embodiments, the first antigen binding domain that binds phosphorylated CRTC3 and/or the second antigen binding domain that binds the second antigen comprise the Fv.

In some of any one of the above- or below-mentioned embodiments, the first antigen binding domain that binds phosphorylated CRTC3 and/or the second antigen binding domain that binds the second antigen comprise the Fd.

In some of any one of the above- or below-mentioned embodiments, the first antigen binding domain that binds phosphorylated CRTC3 and/or the second antigen binding domain that binds the second antigen comprise the scFv.

In some of any one of the above- or below-mentioned embodiments, the scFv comprises, from the N- to C-terminus, a VH, a first linker (L1) and a VL (VH-L1-VL) or the VL, the L1 and the VH (VL-L1-VH). In some of any one of the above- or below-mentioned embodiments, the L1 comprises about 5-50 amino acids. In some of any one of the above- or below-mentioned embodiments, the L1 comprises about 5-40 amino acids. In some of any one of the above- or below-mentioned embodiments, the L1 comprises about 10-30 amino acids. In some of any one of the above- or below-mentioned embodiments, the L1 comprises about 10-20 amino acids. In some of any one of the above- or below-mentioned embodiments, the L1 comprises the amino acid sequence of SEQ ID NOs: 146-178 (Table 4).

In some of any one of the above- or below-mentioned embodiments, the first antigen binding domain that binds phosphorylated CRTC3 is conjugated to a first immunoglobulin (Ig) constant region or a fragment of the first Ig constant region and/or the second antigen binding domain that binds the second antigen is conjugated to a second immunoglobulin (Ig) constant region or a fragment of the second Ig constant region.

In some of any one of the above- or below-mentioned embodiments, the fragment of the first Ig constant region and/or the fragment of the second Ig constant region comprises a Fc region.

In some of any one of the above- or below-mentioned embodiments, the fragment of the first Ig constant region and/or the fragment of the second Ig constant region comprises a CH2 domain.

In some of any one of the above- or below-mentioned embodiments, the fragment of the first Ig constant region and/or the fragment of the second Ig constant region comprises a CH3 domain.

In some of any one of the above- or below-mentioned embodiments, the fragment of the first Ig constant region and/or the fragment of the second Ig constant region comprises the CH2 domain and the CH3 domain.

In some of any one of the above- or below-mentioned embodiments, the fragment of the first Ig constant region and/or the fragment of the second Ig constant region comprises at least portion of a hinge, the CH2 domain and the CH3 domain.

In some of any one of the above- or below-mentioned embodiments, the fragment of the Ig constant region comprises the hinge, the CH2 domain and the CH3 domain.

In some of any one of the above- or below-mentioned embodiments, the multispecific protein further comprises a second linker (L2) between the first antigen binding domain that binds CRTC3 and the first Ig constant region or the fragment of the first Ig constant region and the second antigen binding domain that binds the second antigen and the second Ig constant region or the fragment of the second Ig constant region.

In some of any one of the above- or below-mentioned embodiments, the L2 comprises the amino acid sequence of SEQ ID NOs: 146-178 (Table 4).

In some of any one of the above- or below-mentioned embodiments, the first Ig constant region or the fragment of the first Ig constant region and the second Ig constant region or the fragment of the second Ig constant region is an IgG1, an IgG2, and IgG3 or an IgG4 isotype.

In some of any one of the above- or below-mentioned embodiments, the first Ig constant region or the fragment of the first Ig constant region and the second Ig constant region or the fragment of the second Ig constant region is an IgG1 isotype.

In some of any one of the above- or below-mentioned embodiments, the first Ig constant region or the fragment of the first Ig constant region and the second Ig constant region or the fragment of the second Ig constant region is an IgG2 isotype.

In some of any one of the above- or below-mentioned embodiments, the first Ig constant region or the fragment of the first Ig constant region and the second Ig constant region or the fragment of the second Ig constant region is an IgG3 isotype.

In some of any one of the above- or below-mentioned embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein.

In some of any one of the above- or below-mentioned embodiments, the first Ig constant region or the fragment of the first Ig constant region and the second Ig constant region or the fragment of the second Ig constant region comprises at least one mutation that results in reduced binding of the multispecific protein to a FcγR.

In some of any one of the above- or below-mentioned embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some of any one of the above- or below-mentioned embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some of any one of the above- or below-mentioned embodiments, the at least one mutation that results in enhanced binding of the multispecific protein to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index.

In some of any one of the above- or below-mentioned embodiments, the FcγR is FcγRI, FcγRIIA, FcγRIIB or FcγRIII, or any combination thereof.

In some of any one of the above- or below-mentioned embodiments, the first Ig constant region or the fragment of the first Ig constant region and the second Ig constant region or the fragment of the second Ig constant region comprises at least one mutation that modulates a half-life of the multispecific protein.

In some of any one of the above- or below-mentioned embodiments, the at least one mutation that modulates the half-life of the multispecific protein is selected from the group consisting of H435A, P257I/N434H, D376V/N434H, M252Y/S254T/T256E/H433K/N434F, T308P/N434A and H435R, wherein residue numbering is according to the EU index.

In some of any one of the above- or below-mentioned embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region.

In some of any one of the above- or below-mentioned embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some of any one of the above- or below-mentioned embodiments, the first Ig constant region or the fragment of the first Ig constant region and the second Ig constant region or the fragment of the second Ig constant region comprise the following mutations: L235A_L235A_D265S_T350V_L351Y_F405A_Y407V in the first Ig constant region and L235A_L235A_D265S_T350V_T366L_K392L_T394W in the second Ig constant region; or L235A_L235A_D265S_T350V_T366L_K392L_T394W in the first Ig constant region and L235A_L235A_D265S_T350V_L351Y_F405A_Y407V in the second Ig constant region.

Generation of Multispecific Proteins that Comprise Antigen Binding Fragments that Bind Phosphorylated CRTC3

The antigen binding fragments that bind phosphorylated CRTC3 of the disclosure may be engineered into multispecific antibodies which are also encompassed within the scope of the invention.

The antigen binding fragments that bind phosphorylated CRTC3 may be engineered into full length multispecific antibodies which are generated using Fab arm exchange, in which substitutions are introduced into two monospecific bivalent antibodies within the Ig constant region CH3 domain which promote Fab arm exchange in vitro. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g. Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used are R409D_K370E/D399K_E357K, S354C T366W/Y349C T366S_L368A_Y407V, Y349C T366W/S354C T366S_L368A_Y407V, T366K/L35ID, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding domains that bind phosphorylated CRTC3 can be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The antigen binding domains that bind phosphorylated CRTC3 of the disclosure may also be engineered into multispecific proteins which comprise three polypeptide chains. In such designs, at least one antigen binding domain is in the form of a scFv. Exemplary designs include (in which “1” indicates the first antigen binding domain, “2” indicates the second antigen binding domain and “3” indicates the third antigen binding domain:
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3
Design 4: Chain A) CH2-CH3-scFv1; Chain B) VL2-CL; Chain C) VH2-CH1-hinge-CH2-CH3

CH3 engineering may be incorporated to the Designs 1-4, such as mutations L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

### Conjugation to Immunoglobulin (Ig) Constant Regions or Fragments of the Ig Constant Regions

In some of any one of the above- or below-mentioned embodiments, the antigen binding domains that bind phosphorylated CRTC3 of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding domains that bind phosphorylated CRTC3 of the disclosure may be engineered into conventional full length antibodies using standard methods. The full length antibodies comprising the antigen binding domain that binds phosphorylated CRTC3 may further be engineered as described herein.

In some of any one of the above- or below-mentioned embodiments, an immunoglobulin heavy chain constant region is comprised of subdomains CH1, hinge, CH2 and CH3. In some of any one of the above- or below-mentioned embodiments, the CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some of any one of the above- or below-mentioned embodiments, the Ig Fc region comprises at least the CH2 and the CH3 domains of the Ig constant region, and therefore comprises at least a region from about A231 to K447 of Ig heavy chain constant region.

The invention also provides an antigen binding domain that binds phosphorylated CRTC3 conjugated to an immunoglobulin (Ig) constant region or a fragment of the Ig constant region.

In some of any one of the above- or below-mentioned embodiments, the Ig constant region is a heavy chain constant region.

In some of any one of the above- or below-mentioned embodiments, the Ig constant region is a light chain constant region.

In some of any one of the above- or below-mentioned embodiments, the fragment of the Ig constant region comprises a Fc region.

In some of any one of the above- or below-mentioned embodiments, the fragment of the Ig constant region comprises a CH2 domain.

In some of any one of the above- or below-mentioned embodiments, the fragment of the Ig constant region comprises a CH3 domain.

In some of any one of the above- or below-mentioned embodiments, the fragment of the Ig constant region comprises the CH2 domain and the CH3 domain.

In some of any one of the above- or below-mentioned embodiments, the fragment of the Ig constant region comprises at least portion of a hinge, the CH2 domain and the CH3 domain. Portion of the hinge refers to one or more amino acid residues of the Ig hinge.

In some of any one of the above- or below-mentioned embodiments, the fragment of the Ig constant region comprises the hinge, the CH2 domain and the CH3 domain.

In some of any one of the above- or below-mentioned embodiments, the antigen binding domain that binds phosphorylated CRTC3 is conjugated to the N-terminus of the Ig constant region or the fragment of the Ig constant region.

In some of any one of the above- or below-mentioned embodiments, the antigen binding domain that binds phosphorylated CRTC3 is conjugated to the C-terminus of the Ig constant region or the fragment of the Ig constant region.

In some of any one of the above- or below-mentioned embodiments, the antigen binding domain that binds phosphorylated CRTC3 is conjugated to the Ig constant region or the fragment of the Ig constant region via a second linker (L2).

In some of any one of the above- or below-mentioned embodiments, the L2 comprises the amino acid sequence of SEQ ID NOs: 146-178 (Table 4).

The antigen binding domains that binds phosphorylated CRTC3 of the disclosure conjugated to Ig constant region or the fragment of the Ig constant region may be assessed for their functionality using several known assays. Binding to phosphorylated CRTC3 may be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC may be assessed using an in vitro assay using phosphorylated CRTC3 expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP may be evaluated by using monocyte-derived macrophages as effector cells and any CRTC3 expressing cells as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11+CD14+ macrophages using standard methods.

CDC of cells may be measured for example by plating Daudi cells at 1×10′ cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

### Glycoengineering

The ability of the antigen binding domain that binds phosphorylated CRTC3 conjugated to the Ig constant region or to the fragment of the Ig constant region to mediate ADCC can be enhanced by engineering the Ig constant region or the fragment of the Ig constant region oligosaccharide component. Human IgG1 or IgG3 are N-glycosylated at Asn297 with the majority of the glycans in the well-known biantennary G0, G0F, G1, G1F, G2 or G2F forms. Ig constant region containing proteins may be produced by non-engineered CHO cells typically have a glycan fucose content of about at least 85%. The removal of the core fucose from the biantennary complex-type oligosaccharides attached to the antigen binding domain that binds phosphorylated CRTC3 conjugated to the Ig constant region or to the fragment of the Ig constant region enhances the ADCC of the protein via improved FcγRIIIa binding without altering antigen binding or CDC activity. Such proteins can be achieved using different methods reported to lead to the successful expression of relatively high defucosylated immunoglobulins bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4): 405-415, 2010; PMID:20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or coexpression of 0-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some of any one of the above- or below-mentioned embodiments, the antigen binding domain that binds phosphorylated CRTC3 conjugated to the Ig constant region or to the fragment of the Ig constant region of the disclosure has a biantennary glycan structure with fucose content of about between 1% to about 15%, for example about 15%, 14%, 13%, 12%, 11% 10%, 9%, 8%, 7%, 6%, 5%, 4%, 3%, 2% or 1%. In some of any one of the above- or below-mentioned embodiments, the antigen binding domain that binds CRTC3 conjugated to the Ig constant region or to the fragment of the Ig constant region has a glycan structure with fucose content of about 50%, 40%, 45%, 40%, 35%, 30%, 25%, 20% or any content in between a range defined by any two aforementioned values.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to the antigen binding domain that bind phosphorylated CRTC3 conjugated to the Ig constant region or to the fragment of the Ig constant region with fucose content of about between 1%-15%.

“Normal fucose” or “normal fucose content” as used herein refers to the antigen binding domain that bind phosphorylated CRTC3 conjugated to the Ig constant region or to the fragment of the Ig constant region with fucose content of about over 50%, typically about over 80% or over 85%.

### Anti-Idiotypic Antibodies

Anti-idiotypic antibodies are antibodies that specifically bind to the antigen binding domain that binds phosphorylated CRTC3 of the disclosure.

The disclosure also provides an anti-idiotypic antibody that specifically binds to the antigen binding domain that binds phosphorylated CRTC3 of the disclosure.

The disclosure also provides an anti-idiotypic antibody that specifically binds to the antigen binding domain that binds phosphorylated CRTC3 comprising VH of SEQ ID NOs: 31, 65, 99, or 133; and the VL of SEQ ID NOs: 32, 66, 100, or 134. In one embodiment, the anti-idiotypic antibody that specifically binds to the antigen binding domain that binds phosphorylated CRTC3 comprising the VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antigen binding domain in a sample (e.g. the antigen binding domain that binds phosphorylated CRTC3 of the disclosure). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original antigen binding domain which induced the anti-Id. Thus, by using antibodies to the idiotypic determinants of the antigen binding domain, it is possible to identify other clones expressing antigen binding domains of identical specificity. Anti-Id antibodies may be varied (thereby producing anti-Id antibody variants) and/or derivatized by any suitable technique, such as those described elsewhere herein.

### Immunoconjugates

The antigen binding domains that bind phosphorylated CRTC3 of the disclosure, the proteins comprising the antigen binding domains that bind phosphorylated CRTC3 (collectively referred herein as to phosphorylated CRTC3 binding proteins) may be conjugated to a heterologous molecule.

In some of any one of the above- or below-mentioned embodiments, the heterologous molecule is a detectable label or a cytotoxic agent.

The invention also provides an antigen binding domain that binds phosphorylated CRTC3 conjugated to a detectable label.

The invention also provides a protein comprising an antigen binding domain that binds phosphorylated CRTC3 conjugated to a detectable label.

The invention also provides a multispecific protein comprising an antigen binding domain that binds phosphorylated CRTC3 conjugated to a detectable label.

The invention also provides an antigen binding domain that binds phosphorylated CRTC3 conjugated to a cytotoxic agent.

The invention also provides a protein comprising an antigen binding domain that binds phosphorylated CRTC3 conjugated to a cytotoxic agent.

The invention also provides a multispecific protein comprising an antigen binding domain that binds phosphorylated CRTC3 conjugated to a cytotoxic agent.

The phosphorylated CRTC3 binding proteins of the disclosure may be used to direct therapeutics to phosphorylated CRTC3-expressing cells.

In some of any one of the above- or below-mentioned embodiments, the detectable label is also a cytotoxic agent.

The CRTC3 binding proteins of the disclosure conjugated to a detectable label may be used to evaluate expression of phosphorylated CRTC3 on a variety of samples.

Detectable label includes compositions that when conjugated to the phosphorylated CRTC3 binding proteins of the disclosure renders the latter detectable, via spectroscopic, photochemical, biochemical, immunochemical, or chemical means.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni2+, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include 3H, 11C, 13C, 15N, 18F, 19F, 55Co, 57Co, 60Co, 61Cu, 62Cu, 64Cu, 67Cu, 68Ga, 72As, 75Br, 86Y, 89Zr, 90Sr, 94mTc, 99mTc, 115In, 1231, 1241, 125I, 1311, 211At, 212Bi, 213Bi, 223Ra, 226Ra, 225Ac and 227Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, leand atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some of any one of the above- or below-mentioned embodiments, the metal atoms may be alkaline earth metals with an atomic number greater than twenty.

In some of any one of the above- or below-mentioned embodiments, the metal atoms may be lanthanides.

In some of any one of the above- or below-mentioned embodiments, the metal atoms may be actinides.

In some of any one of the above- or below-mentioned embodiments, the metal atoms may be transition metals.

In some of any one of the above- or below-mentioned embodiments, the metal atoms may be poor metals.

In some of any one of the above- or below-mentioned embodiments, the metal atoms may be gold atoms, bismuth atoms, tantalum atoms, and gadolinium atoms.

In some of any one of the above- or below-mentioned embodiments, the metal atoms may be metals with an atomic number of 53 (i.e. iodine) to 83 (i.e. bismuth).

In some of any one of the above- or below-mentioned embodiments, the metal atoms may be atoms suitable for magnetic resonance imaging.

The metal atoms may be metal ions in the form of +1, +2, or +3 oxidation states, such as Ba2+, Bi3+, Cs+, Ca2+, Cr2+, Cr3+, Cr6+, Co2+, Co3+, Cu+, Cu2+, Cu3+, Ga3+, Gd3+, Au+, Au3+, Fe2+, Fe3+, F3+, Pb2+, Mn2+, Mn3+, Mn4+, Mn7+, Hg2+, Ni2+, Ni3+, Ag+, Sr2+, Sn2+, Sn4+, and Zn2+. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The antigen binding domain that binds phosphorylated CRTC3 conjugated to a detectable label may be used as an imaging agent.

The protein comprising an antigen binding domain that binds phosphorylated CRTC3 conjugated to a detectable label may be used as an imaging agent.

The multispecific protein comprising an antigen binding domain that binds phosphorylated CRTC3 conjugated to a detectable label may be used as an imaging agent.

In some of any one of the above- or below-mentioned embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some of any one of the above- or below-mentioned embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some of any one of the above- or below-mentioned embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some of any one of the above- or below-mentioned embodiments, the cytotoxic agent is a radionuclide, such as 212Bi, 1311, 131In, 90Y, and 186Re.

In some of any one of the above- or below-mentioned embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the invention through the N (amino) terminus or the C (carboxyl) terminus of the peptidic drug moiety (WO02/088172), or via any cysteine engineered into the antibody.

The phosphorylated CRTC3 binding proteins of the disclosure may be conjugated to a detectable label using known methods.

In some of any one of the above- or below-mentioned embodiments, the detectable label is complexed with a chelating agent.

In some of any one of the above- or below-mentioned embodiments, the detectable label is conjugated to the phosphorylated CRTC3 binding proteins of the disclosure via a linker.

The detectable label or the cytotoxic moiety may be linked directly, or indirectly, to the CRTC3 binding proteins of the disclosure using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some of any one of the above- or below-mentioned embodiments, the phosphorylated CRTC3 binding proteins of the disclosure is removed from the blood via renal clearance.

### Kits

The invention also provides a kit comprising the antigen binding domain that binds phosphorylated CRTC3.

The invention also provides a kit comprising the protein comprising an antigen binding domain that binds phosphorylated CRTC3.

The invention also provides a kit comprising the multispecific protein comprising an antigen binding domain that binds phosphorylated CRTC3.

The kit may be used for therapeutic uses and as diagnostic kits.

The kit may be used to detect the presence of phosphorylated CRTC3 in a sample.

In some of any one of the above- or below-mentioned embodiments, the kit comprises the phosphorylated CRTC3 binding protein of the disclosure and reagents for detecting the phosphorylated CRTC3 binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some of any one of the above- or below-mentioned embodiments, the kit comprises the antigen binding domain that binds phosphorylated CRTC3 in a container and instructions for use of the kit.

In some of any one of the above- or below-mentioned embodiments, the kit comprises the protein comprising an antigen binding domain that binds phosphorylated CRTC3 in a container and instructions for use of the kit.

In some of any one of the above- or below-mentioned embodiments, the kit comprises the multispecific protein comprising an antigen binding domain that binds phosphorylated CRTC3 in a container and instructions for use of the kit.

In some of any one of the above- or below-mentioned embodiments, the antigen binding domain that binds phosphorylated CRTC3 in the kit is labeled.

In some of any one of the above- or below-mentioned embodiments, the protein comprising an antigen binding domain that binds phosphorylated CRTC3 in the kit is labeled.

In some of any one of the above- or below-mentioned embodiments, the multispecific protein comprising an antigen binding domain that binds phosphorylated CRTC3 in the kit is labeled.

In some of any one of the above- or below-mentioned embodiments, the kit comprises the antigen binding domain that binds phosphorylated CRTC3 comprising a VH of SEQ ID NOs: 31, 65, 99, or 133; and the VL of SEQ ID NOs: 32, 66, 100, or 134. In some of any one of the above- or below-mentioned embodiments, the kit comprises the antigen binding domain that binds CRTC3 comprising a VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

### Polynucleotides, Host Cells and Vectors

The disclosure also provides an isolated polynucleotide encoding any of the phosphorylated CRTC3 binding proteins of the disclosure. The phosphorylated CRTC3 binding protein includes the antigen binding domains that bind phosphorylated CRTC3, the proteins comprising the antigen binding domains that bind phosphorylated CRTC3, and the multispecific proteins that comprise the antigen binding domains that bind phosphorylated CRTC3.

The disclosure also provides an isolated polynucleotide encoding any of phosphorylated CRTC3 binding proteins or fragments thereof.

mAb #157

In one embodiment, the isolated polynucleotide encodes a phosphorylated CRTC3 binding protein comprising a heavy chain complementarity determining region (HCDR) 1 of SEQ ID NOs: 1, 7, 13, 19 or 25.

In one embodiment, the isolated polynucleotide encodes a phosphorylated CRTC3 binding protein comprising a HCDR2 of SEQ ID NOs: 2, 8, 14, 20 or 26.

In one embodiment, the isolated polynucleotide encodes a phosphorylated CRTC3 binding protein comprising a HCDR3 of SEQ ID NOs: 3, 9, 15, 21 or 27.

In one embodiment, the isolated polynucleotide encodes a phosphorylated CRTC3 binding protein comprising a HDCR1 of SEQ ID NOs: 1, 7, 13, 19 or 25; a HCDR2 of SEQ ID NOs: 2, 8, 14, 20 or 26; and a HCDR3 of SEQ ID NOs: 3, 9, 15, 21 or 27.

In one embodiment, the isolated polynucleotide encodes a phosphorylated CRTC3 binding protein comprising a HCDR1, HCDR2 and HCDR3 of:
SEQ ID NOs: 1, 2, and 3, respectively;
SEQ ID NOs: 4, 5, and 6, respectively;
SEQ ID NOs: 7, 8, and 9, respectively;
SEQ ID NOs: 10, 11, and 12, respectively; or
SEQ ID NOs: 13, 14, and 15, respectively.

In one embodiment, the isolated polynucleotide encodes a phosphorylated CRTC3 binding protein comprising a light chain complementarity determining region (LCDR) 1 of SEQ ID NOs: 4, 22, or 28.

In one embodiment, the isolated polynucleotide encodes a phosphorylated CRTC3 binding protein comprising a LCDR2 of SEQ ID NOs: 5, 23, or 29.

In one embodiment, the isolated polynucleotide encodes a phosphorylated CRTC3 binding protein comprising a LCDR3 of SEQ ID NOs: 6, 24, or 30.

In one embodiment, the isolated polynucleotide encodes a phosphorylated CRTC3 binding protein comprising a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the isolated polynucleotide encodes a phosphorylated CRTC3 binding protein comprising a LCDR1, LCDR2 and LCDR3 of:
SEQ ID NOs: 16, 17, and 18, respectively;
SEQ ID NOs: 19, 20, and 21, respectively;
SEQ ID NOs: 22, 23, and 24, respectively;
SEQ ID NOs: 25, 26, and 27, respectively; or
SEQ ID NOs: 28, 29, and 30, respectively.

In one embodiment, the isolated polynucleotide encodes a phosphorylated CRTC3 binding protein comprising a HDCR1 of SEQ ID NOs: 1, 7, 13, 19 or 25; a HCDR2 of SEQ ID NOs: 2, 8, 14, 20 or 26; a HCDR3 of SEQ ID NOs: 3, 9, 15, 21 or 27; a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the isolated polynucleotide encodes a phosphorylated CRTC3 binding protein comprising a HCDR1, HCDR2, HCDR3, LCDR1, LCDR2 and LCDR3 of:
SEQ ID NOs: 1, 2, 3, 16, 17, and 18, respectively;
SEQ ID NOs: 4, 5, 6, 19, 20, and 21, respectively;
SEQ ID NOs: 7, 8, 9, 22, 23, and 24, respectively;
SEQ ID NOs: 10, 11, 12, 25, 26, and 27, respectively; or
SEQ ID NOs: 13, 14, 15, 28, 29, and 30, respectively.

In one embodiment, the isolated polynucleotide encoding the VH of a phosphorylated CRTC3 binding protein comprises a nucleotide sequence of SEQ ID NO: 179.

In one embodiment, the isolated polynucleotide encoding the VL of a phosphorylated CRTC3 binding protein comprises a nucleotide sequence of SEQ ID NO: 180.

mAb #170

In one embodiment, the isolated polynucleotide encodes a phosphorylated CRTC3 binding protein comprising a heavy chain complementarity determining region (HCDR) 1 of SEQ ID NOs: 35, 41, 47, 53 or 59.

In one embodiment, the isolated polynucleotide encodes a phosphorylated CRTC3 binding protein comprising a HCDR2 of SEQ ID NOs: 36, 42, 48, 54 or 60.

In one embodiment, the isolated polynucleotide encodes a phosphorylated CRTC3 binding protein comprising a HCDR3 of SEQ ID NOs: 37, 43, 49, 55 or 61.

In one embodiment, the isolated polynucleotide encodes a phosphorylated CRTC3 binding protein comprising a HDCR1 of SEQ ID NOs: 35, 41, 47, 53 or 59; a HCDR2 of SEQ ID NOs: 36, 42, 48, 54 or 60; and a HCDR3 of SEQ ID NOs: 37, 43, 49, 55 or 61.

In one embodiment, the isolated polynucleotide encodes a phosphorylated CRTC3 binding protein comprising a HCDR1, HCDR2 and HCDR3 of:
SEQ ID NOs: 35, 36, and 37, respectively;
SEQ ID NOs: 38, 39, and 40, respectively;
SEQ ID NOs: 41, 42, and 43, respectively;
SEQ ID NOs: 44, 45, and 46, respectively; or
SEQ ID NOs: 47, 48, and 49, respectively.

In one embodiment, the isolated polynucleotide encodes a phosphorylated CRTC3 binding protein comprising a light chain complementarity determining region (LCDR) 1 of SEQ ID NOs: 38, 56, or 62.

In one embodiment, the isolated polynucleotide encodes a phosphorylated CRTC3 binding protein comprising a LCDR2 of SEQ ID NOs: 39, 57, or 63.

In one embodiment, the isolated polynucleotide encodes a phosphorylated CRTC3 binding protein comprising a LCDR3 of SEQ ID NOs: 40, 58, or 64.

In one embodiment, the isolated polynucleotide encodes a phosphorylated CRTC3 binding protein comprising a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the isolated polynucleotide encodes a phosphorylated CRTC3 binding protein comprising a LCDR1, LCDR2 and LCDR3 of:
SEQ ID NOs: 50, 51, and 52, respectively;
SEQ ID NOs: 53, 54, and 55, respectively;
SEQ ID NOs: 56, 57, and 58, respectively;
SEQ ID NOs: 59, 60, and 61, respectively; or
SEQ ID NOs: 62, 63, and 64, respectively.

In one embodiment, the isolated polynucleotide encodes a phosphorylated CRTC3 binding protein comprising a HDCR1 of SEQ ID NOs: 35, 41, 47, 53 or 59; a HCDR2 of SEQ ID NOs: 36, 42, 48, 54 or 60; a HCDR3 of SEQ ID NOs: 37, 43, 49, 55 or 61; a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the isolated polynucleotide encodes a phosphorylated CRTC3 binding protein comprising a HCDR1, HCDR2, HCDR3, LCDR1, LCDR2 and LCDR3 of:
SEQ ID NOs: 35, 36, 37, 50, 51, and 52, respectively;
SEQ ID NOs: 38, 39, 40, 53, 54, and 55, respectively;
SEQ ID NOs: 41, 42, 43, 56, 57, and 58, respectively;
SEQ ID NOs: 44, 45, 46, 59, 60, and 61, respectively; or
SEQ ID NOs: 47, 48, 49, 62, 63, and 64, respectively.

In one embodiment, the isolated polynucleotide encoding the VH of a phosphorylated CRTC3 binding protein comprises a nucleotide sequence of SEQ ID NO: 181.

In one embodiment, the isolated polynucleotide encoding the VL of a phosphorylated CRTC3 binding protein comprises a nucleotide sequence of SEQ ID NO: 182.

mAb #21

In one embodiment, the isolated polynucleotide encodes a phosphorylated CRTC3 binding protein comprising a heavy chain complementarity determining region (HCDR) 1 of SEQ ID NOs: 69, 75, 81, 87, or 93.

In one embodiment, the isolated polynucleotide encodes a phosphorylated CRTC3 binding protein comprising a HCDR2 of SEQ ID NOs: 70, 76, 82, 88, or 94.

In one embodiment, the isolated polynucleotide encodes a phosphorylated CRTC3 binding protein comprising a HCDR3 of SEQ ID NOs: 71, 77, 83, 89, or 95.

In one embodiment, the isolated polynucleotide encodes a phosphorylated CRTC3 binding protein comprising a HDCR1 of SEQ ID NOs: 69, 75, 81, 87, or 93; a HCDR2 of SEQ ID NOs: 70, 76, 82, 88, or 94; and a HCDR3 of SEQ ID NOs: 71, 77, 83, 89, or 95.

In one embodiment, the isolated polynucleotide encodes a phosphorylated CRTC3 binding protein comprising a HCDR1, HCDR2 and HCDR3 of:
SEQ ID NOs: 69, 70, and 71, respectively;
SEQ ID NOs: 72, 73, and 74, respectively;
SEQ ID NOs: 75, 76, and 77, respectively;
SEQ ID NOs: 78, 79, and 80, respectively; or
SEQ ID NOs: 81, 82, and 83, respectively.

In one embodiment, the isolated polynucleotide encodes a phosphorylated CRTC3 binding protein comprising a light chain complementarity determining region (LCDR) 1 of SEQ ID NOs: 72, 90, or 96.

In one embodiment, the isolated polynucleotide encodes a phosphorylated CRTC3 binding protein comprising a LCDR2 of SEQ ID NOs: 73, 91, or 97.

In one embodiment, the isolated polynucleotide encodes a phosphorylated CRTC3 binding protein comprising a LCDR3 of SEQ ID NOs: 74, 92, or 98.

In one embodiment, the isolated polynucleotide encodes a phosphorylated CRTC3 binding protein comprising a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the isolated polynucleotide encodes a phosphorylated CRTC3 binding protein comprising a LCDR1, LCDR2 and LCDR3 of:
SEQ ID NOs: 84, 85, and 86, respectively;
SEQ ID NOs: 87, 88, and 89, respectively;
SEQ ID NOs: 90, 91, and 92, respectively;
SEQ ID NOs: 93, 94, and 95, respectively; or
SEQ ID NOs: 96, 97, and 98, respectively.

In one embodiment, the isolated polynucleotide encodes a phosphorylated CRTC3 binding protein comprising a HDCR1 of SEQ ID NOs: 69, 75, 81, 87, or 93; a HCDR2 of SEQ ID NOs: 70, 76, 82, 88, or 94; a HCDR3 of SEQ ID NOs: 71, 77, 83, 89, or 95; a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the isolated polynucleotide encodes a phosphorylated CRTC3 binding protein comprising a HCDR1, HCDR2, HCDR3, LCDR1, LCDR2 and LCDR3 of:
SEQ ID NOs: 69, 70, 71, 84, 85, and 86, respectively;
SEQ ID NOs: 72, 73, 74, 87, 88, and 89, respectively;
SEQ ID NOs: 75, 76, 77, 90, 91, and 92, respectively;
SEQ ID NOs: 78, 79, 80, 93, 94, and 95, respectively; or
SEQ ID NOs: 81, 82, 83, 96, 97, and 98, respectively.

In one embodiment, the isolated polynucleotide encoding the VH of a phosphorylated CRTC3 binding protein comprises a nucleotide sequence of SEQ ID NO: 183.

In one embodiment, the isolated polynucleotide encoding the VL of a phosphorylated CRTC3 binding protein comprises a nucleotide sequence of SEQ ID NO: 184.

mAb #82

In one embodiment, the isolated polynucleotide encodes a phosphorylated CRTC3 binding protein comprising a heavy chain complementarity determining region (HCDR) 1 of SEQ ID NOs: 103, 109, 115, 121, or 127.

In one embodiment, the isolated polynucleotide encodes a phosphorylated CRTC3 binding protein comprising a HCDR2 of SEQ ID NOs: 104, 110, 116, 122, or 128.

In one embodiment, the isolated polynucleotide encodes a phosphorylated CRTC3 binding protein comprising a HCDR3 of SEQ ID NOs: 105, 111, 117, 123, or 129.

In one embodiment, the isolated polynucleotide encodes a phosphorylated CRTC3 binding protein comprising a HDCR1 of SEQ ID NOs: 103, 109, 115, 121, or 127; a HCDR2 of SEQ ID NOs: 104, 110, 116, 122, or 128; and a HCDR3 of SEQ ID NOs: 105, 111, 117, 123, or 129.

In one embodiment, the isolated polynucleotide encodes a phosphorylated CRTC3 binding protein comprising a HCDR1, HCDR2 and HCDR3 of:
SEQ ID NOs: 103, 104, and 105, respectively;
SEQ ID NOs: 106, 107, and 108, respectively;
SEQ ID NOs: 109, 110, and 111, respectively;
SEQ ID NOs: 112, 113, and 114, respectively; or
SEQ ID NOs: 115, 116, and 117, respectively.

In one embodiment, the isolated polynucleotide encodes a phosphorylated CRTC3 binding protein comprising a light chain complementarity determining region (LCDR) 1 of SEQ ID NOs: 106, 124, or 130.

In one embodiment, the isolated polynucleotide encodes a phosphorylated CRTC3 binding protein comprising a LCDR2 of SEQ ID NOs: 107, 125, or 131.

In one embodiment, the isolated polynucleotide encodes a phosphorylated CRTC3 binding protein comprising a LCDR3 of SEQ ID NOs: 108, 126, or 132.

In one embodiment, the isolated polynucleotide encodes a phosphorylated CRTC3 binding protein comprising a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the isolated polynucleotide encodes a phosphorylated CRTC3 binding protein comprising a LCDR1, LCDR2 and LCDR3 of:
SEQ ID NOs: 118, 119, and 120, respectively;
SEQ ID NOs: 121, 122, and 123, respectively;
SEQ ID NOs: 124, 125, and 126, respectively;
SEQ ID NOs: 127, 128, and 129, respectively; or
SEQ ID NOs: 130, 131, and 132, respectively.

In one embodiment, the isolated polynucleotide encodes a phosphorylated CRTC3 binding protein comprising a HDCR1 of SEQ ID NOs: 103, 109, 115, 121, or 127; a HCDR2 of SEQ ID NOs: 104, 110, 116, 122, or 128; a HCDR3 of SEQ ID NOs: 105, 111, 117, 123, or 129; a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the isolated polynucleotide encodes a phosphorylated CRTC3 binding protein comprising a HCDR1, HCDR2, HCDR3, LCDR1, LCDR2 and LCDR3 of:
SEQ ID NOs: 103, 104, 105, 118, 119, and 120, respectively;
SEQ ID NOs: 106, 107, 108, 121, 122, and 123, respectively;
SEQ ID NOs: 109, 110, 111, 124, 125, and 126, respectively;
SEQ ID NOs: 112, 113, 114, 127, 128, and 129, respectively; or
SEQ ID NOs: 115, 116, 117, 130, 131, and 132, respectively.

In one embodiment, the isolated polynucleotide encoding the VH of a phosphorylated CRTC3 binding protein comprises a nucleotide sequence of SEQ ID NO: 185.

In one embodiment, the isolated polynucleotide encoding the VL of a phosphorylated CRTC3 binding protein comprises a nucleotide sequence of SEQ ID NO: 186.

In one embodiment, the isolated polynucleotide encodes a phosphorylated CRTC3 binding protein comprising a VH of SEQ ID NOs: 31, 65, 99, or 133.

In one embodiment, the isolated polynucleotide encodes a phosphorylated CRTC3 binding protein comprising a VL of SEQ ID NOs: 32, 66, 100, or 134.

In one embodiment, the isolated polynucleotide encodes a phosphorylated CRTC3 binding protein comprising a VH of SEQ ID NOs: 31, 65, 99, or 133; and the VL of SEQ ID NOs: 32, 66, 100, or 134.

In one embodiment, the isolated polynucleotide encodes a phosphorylated CRTC3 binding protein comprising a VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

In one embodiment, the isolated polynucleotide encoding a phosphorylated CRTC3 binding protein comprises a nucleotide sequence of SEQ ID NOs: 179, 181, 183 or 185; and a nucleotide sequence of SEQ ID NOs: 180, 182, 184, or 186.

In one embodiment, the isolated polynucleotide encoding a phosphorylated CRTC3 binding protein comprises at least two nucleotide sequences of:
SEQ ID NOs: 179 and 180;
SEQ ID NOs: 181 and 182;
SEQ ID NOs: 183 and 184; or
SEQ ID NOs: 185 and 186.

In one embodiment, the isolated polynucleotide encodes a phosphorylated CRTC3 binding protein comprising a heavy chain (HC) of SEQ ID NOs: 33, 67, 101, or 135.

In one embodiment, the isolated polynucleotide encodes a phosphorylated CRTC3 binding protein comprising a light chain (LC) of SEQ ID NOs: 34, 68, 102, or 136.

In one embodiment, the isolated polynucleotide encodes a phosphorylated CRTC3 binding protein comprising a HC of SEQ ID NOs: 33, 67, 101, or 135; and a LC of SEQ ID NOs: 34, 68, 102, or 136.

In one embodiment, the isolated polynucleotide encodes a phosphorylated CRTC3 binding protein comprising the HC and LC of:
SEQ ID NOs: 33 and 34, respectively;
SEQ ID NOs: 67 and 68, respectively;
SEQ ID NOs: 101 and 102, respectively; or
SEQ ID NOs: 135 and 136, respectively.

Some embodiments of the disclosure also provide an isolated or purified nucleic acid comprising a polynucleotide which is complementary to the polynucleotides encoding the phosphorylated CRTC3 binding proteins of the disclosure or polynucleotides which hybridize under stringent conditions to the polynucleotides encoding the phosphorylated CRTC3 binding proteins of the disclosure.

The polynucleotides which hybridize under stringent conditions may hybridize under high stringency conditions. By “high stringency conditions” is meant that the polynucleotide specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

The polynucleotide sequences of the disclosure may be operably linked to one or more regulatory elements, such as a promoter or enhancer, that allow expression of the nucleotide sequence in the intended host cell. The polynucleotide may be a cDNA. The promoter may be a strong, weak, tissue-specific, inducible or developmental-specific promoter. Exemplary promoters that may be used are hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like may also be used.

The invention also provides a vector comprising the polynucleotide of the invention. The disclosure also provides an expression vector comprising the polynucleotide of the invention. Such vectors may be plasmid vectors, viral vectors, vectors for baculovirus expression, transposon based vectors or any other vector suitable for introduction of the synthetic polynucleotide of the invention into a given organism or genetic background by any means. Polynucleotides encoding the phosphorylated CRTC3 binding proteins of the disclosure may be operably linked to control sequences in the expression vector(s) that ensure the expression of the phosphorylated CRTC3 binding proteins. Such regulatory elements may include a transcriptional promoter, sequences encoding suitable mRNA ribosomal binding sites, and sequences that control the termination of transcription and translation. Expression vectors may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The expression vectors can comprise naturally-occurring or non-naturally-occurring internucleotide linkages, or both types of linkages. The non-naturally occurring or altered nucleotides or internucleotide linkages do not hinder the transcription or replication of the vector.

Once the vector has been incorporated into the appropriate host, the host is maintained under conditions suitable for high level expression of the phosphorylated CRTC3 binding proteins of the disclosure encoded by the incorporated polynucleotides. The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

Vectors of the disclosure may also contain one or more Internal Ribosome Entry Site(s) (IRES). Inclusion of an IRES sequence into fusion vectors may be beneficial for enhancing expression of some proteins. In some of any one of the above- or below-mentioned embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2 plasmid, λ, bovine papilloma virus, and the like.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol x resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif), the pET series (Novagen, Madison, Wis.), the pGEX series (Pharmacia Biotech, Uppsala, Sweden), and the pEX series (Clontech, Palo Alto, Calif). Bacteriophage vectors, such as XGT10, XGT11, XEMBL4, and aNM1149, aZapII (Stratagene) can be used. Exemplary plant expression vectors include pBI01, pBI01.2, pBI121, pBI101.3, and pBIN19 (Clontech). Exemplary animal expression vectors include pEUK-C1, pMAM, and pMAMneo (Clontech). The expression vector may be a viral vector, e.g., a retroviral vector, e.g., a gamma retroviral vector.

mAb #157

In one embodiment, the vector comprises a polynucleotide encoding a heavy chain complementarity determining region (HCDR) 1 of SEQ ID NOs: 1, 7, 13, 19 or 25.

In one embodiment, the vector comprises a polynucleotide encoding a HCDR2 of SEQ ID NOs: 2, 8, 14, 20 or 26.

In one embodiment, the vector comprises a polynucleotide encoding a HCDR3 of SEQ ID NOs: 3, 9, 15, 21 or 27.

In one embodiment, the vector comprises a polynucleotide encoding a HDCR1 of SEQ ID NOs: 1, 7, 13, 19 or 25; a HCDR2 of SEQ ID NOs: 2, 8, 14, 20 or 26; and a HCDR3 of SEQ ID NOs: 3, 9, 15, 21 or 27.

In one embodiment, the vector comprises a polynucleotide encoding a HCDR1, HCDR2 and HCDR3 of:
SEQ ID NOs: 1, 2, and 3, respectively;
SEQ ID NOs: 7, 8, and 9, respectively;
SEQ ID NOs: 13, 14, and 15, respectively;
SEQ ID NOs: 19, 20, and 21, respectively; or
SEQ ID NOs: 25, 26, and 27, respectively.

In one embodiment, the vector comprises a polynucleotide encoding a light chain complementarity determining region (LCDR) 1 of SEQ ID NOs: 4, 22, or 28.

In one embodiment, the vector comprises a polynucleotide encoding a LCDR2 of SEQ ID NOs: 5, 23, or 29.

In one embodiment, the vector comprises a polynucleotide encoding a LCDR3 of SEQ ID NOs: 6, 24, or 30.

In one embodiment, the vector comprises a polynucleotide encoding a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the vector comprises a polynucleotide encoding a LCDR1, LCDR2 and LCDR3 of:
SEQ ID NOs: 4, 5 and 6, respectively;
SEQ ID NOs: 22, 23 and 24, respectively; or
SEQ ID NOs: 28, 29 and 30, respectively.

In one embodiment, the vector comprises a polynucleotide encoding a HDCR1 of SEQ ID NOs: 1, 7, 13, 19 or 25; a HCDR2 of SEQ ID NOs: 2, 8, 14, 20 or 26; a HCDR3 of SEQ ID NOs: 3, 9, 15, 21 or 27; a LDCR1 of SEQ ID NOs: 4, 22, or 28; a LCDR2 of SEQ ID NOs: 5, 23, or 29; and a LCDR3 of SEQ ID NOs: 6, 24, or 30.

In one embodiment, the vector comprises a polynucleotide encoding a HCDR1, HCDR2, HCDR3, LCDR1, LCDR2 and LCDR3 of:
SEQ ID NOs: 1, 2, 3, 4, 5 and 6, respectively;
SEQ ID NOs: 7, 8, 9, 4, 5 and 6, respectively;
SEQ ID NOs: 13, 14, 15, 4, 5 and 6, respectively;
SEQ ID NOs: 19, 20, 21, 22, 23, and 24, respectively; or
SEQ ID NOs: 25, 26, 27, 28, 29, and 30, respectively.

In one embodiment, the vector comprises a polynucleotide encoding the VH of a phosphorylated CRTC3 binding protein comprising a nucleotide sequence of SEQ ID NO: 179.

In one embodiment, the vector comprises a polynucleotide encoding the VL of a phosphorylated CRTC3 binding protein comprising a nucleotide sequence of SEQ ID NO: 180.

mAb #170

In one embodiment, the vector comprises a polynucleotide encoding a heavy chain complementarity determining region (HCDR) 1 of SEQ ID NOs: 35, 41, 47, 53 or 59.

In one embodiment, the vector comprises a polynucleotide encoding a HCDR2 of SEQ ID NOs: 36, 42, 48, 54 or 60.

In one embodiment, the vector comprises a polynucleotide encoding a HCDR3 of SEQ ID NOs: 37, 43, 49, 55 or 61.

In one embodiment, the vector comprises a polynucleotide encoding a HDCR1 of SEQ ID NOs: 35, 41, 47, 53 or 59; a HCDR2 of SEQ ID NOs: 36, 42, 48, 54 or 60; and a HCDR3 of SEQ ID NOs: 37, 43, 49, 55 or 61.

In one embodiment, the vector comprises a polynucleotide encoding a HCDR1, HCDR2 and HCDR3 of:
SEQ ID NOs: 35, 36, and 37, respectively;
SEQ ID NOs: 41, 42, and 43, respectively;
SEQ ID NOs: 47, 48, and 49, respectively;
SEQ ID NOs: 53, 54, and 55, respectively; or
SEQ ID NOs: 59, 60, and 61, respectively.

In one embodiment, the vector comprises a polynucleotide encoding a light chain complementarity determining region (LCDR) 1 of SEQ ID NOs: 38, 56, or 62.

In one embodiment, the vector comprises a polynucleotide encoding a LCDR2 of SEQ ID NOs: 39, 57, or 63.

In one embodiment, the vector comprises a polynucleotide encoding a LCDR3 of SEQ ID NOs: 40, 58, or 64.

In one embodiment, the vector comprises a polynucleotide encoding a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the vector comprises a polynucleotide encoding a LCDR1, LCDR2 and LCDR3 of:
SEQ ID NOs: 38, 39, and 40, respectively;
SEQ ID NOs: 56, 57, and 58, respectively; or
SEQ ID NOs: 62, 63, and 64, respectively.

In one embodiment, the vector comprises a polynucleotide encoding a HDCR1 of SEQ ID NOs: 35, 41, 47, 53 or 59; a HCDR2 of SEQ ID NOs: 36, 42, 48, 54 or 60; a HCDR3 of SEQ ID NOs: 37, 43, 49, 55 or 61; a LDCR1 of SEQ ID NOs: 38, 56, or 62; a LCDR2 of SEQ ID NOs: 39, 57, or 63; and a LCDR3 of SEQ ID NOs: 40, 58, or 64.

In one embodiment, the vector comprises a polynucleotide encoding a HCDR1, HCDR2, HCDR3, LCDR1, LCDR2 and LCDR3 of:
SEQ ID NOs: 35, 36, 37, 38, 39, and 40, respectively;
SEQ ID NOs: 41, 42, 43, 38, 39, and 40, respectively;
SEQ ID NOs: 47, 48, 49, 38, 39, and 40, respectively;
SEQ ID NOs: 53, 54, 55, 56, 57, and 58, respectively; or
SEQ ID NOs: 59, 60, 61, 62, 63, and 64, respectively.

In one embodiment, the vector comprises a polynucleotide encoding the VH of a phosphorylated CRTC3 binding protein comprising a nucleotide sequence of SEQ ID NO: 181.

In one embodiment, the vector comprises a polynucleotide encoding the VL of a phosphorylated CRTC3 binding protein comprising a nucleotide sequence of SEQ ID NO: 182.

mAb #21

In one embodiment, the vector comprises a polynucleotide encoding a heavy chain complementarity determining region (HCDR) 1 of SEQ ID NOs: 69, 75, 81, 87, or 93.

In one embodiment, the vector comprises a polynucleotide encoding a HCDR2 of SEQ ID NOs: 70, 76, 82, 88, or 94.

In one embodiment, the vector comprises a polynucleotide encoding a HCDR3 of SEQ ID NOs: 71, 77, 83, 89, or 95.

In one embodiment, the vector comprises a polynucleotide encoding a HDCR1 of SEQ ID NOs: 69, 75, 81, 87, or 93; a HCDR2 of SEQ ID NOs: 70, 76, 82, 88, or 94; and a HCDR3 of SEQ ID NOs: 71, 77, 83, 89, or 95.

In one embodiment, the vector comprises a polynucleotide encoding a HCDR1, HCDR2 and HCDR3 of:
SEQ ID NOs: 69, 70, and 71, respectively;
SEQ ID NOs: 75, 76, and 77, respectively;
SEQ ID NOs: 81, 82, and 83, respectively;
SEQ ID NOs: 87, 88, and 89, respectively; or
SEQ ID NOs: 93, 94, and 95, respectively.

In one embodiment, the vector comprises a polynucleotide encoding a light chain complementarity determining region (LCDR) 1 of SEQ ID NOs: 72, 90, or 96.

In one embodiment, the vector comprises a polynucleotide encoding a LCDR2 of SEQ ID NOs: 73, 91, or 97.

In one embodiment, the vector comprises a polynucleotide encoding a LCDR3 of SEQ ID NOs: 74, 92, or 98.

In one embodiment, the vector comprises a polynucleotide encoding a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the vector comprises a polynucleotide encoding a LCDR1, LCDR2 and LCDR3 of:
SEQ ID NOs: 72, 73, and 74, respectively;
SEQ ID NOs: 90, 91, and 92, respectively; or
SEQ ID NOs: 96, 97, and 98, respectively.

In one embodiment, the vector comprises a polynucleotide encoding a HDCR1 of SEQ ID NOs: 69, 75, 81, 87, or 93; a HCDR2 of SEQ ID NOs: 70, 76, 82, 88, or 94; a HCDR3 of SEQ ID NOs: 71, 77, 83, 89, or 95; a LDCR1 of SEQ ID NOs: 72, 90, or 96; a LCDR2 of SEQ ID NOs: 73, 91, or 97; and a LCDR3 of SEQ ID NOs: 74, 92, or 98.

In one embodiment, the vector comprises a polynucleotide encoding a HCDR1, HCDR2, HCDR3, LCDR1, LCDR2 and LCDR3 of:
SEQ ID NOs: 69, 70, 71, 72, 73, and 74, respectively;
SEQ ID NOs: 75, 76, 77, 72, 73, and 74, respectively;
SEQ ID NOs: 81, 82, 83, 72, 73, and 74, respectively; SEQ ID NOs: 87, 88, 89, 90, 91, and 92, respectively; or
SEQ ID NOs: 93, 94, 95, 96, 97, and 98 respectively.

In one embodiment, the vector comprises a polynucleotide encoding the VH of a phosphorylated CRTC3 binding protein comprising a nucleotide sequence of SEQ ID NO: 183.

In one embodiment, the vector comprises a polynucleotide encoding the VL of a phosphorylated CRTC3 binding protein comprising a nucleotide sequence of SEQ ID NO: 184.

mAb #82

In one embodiment, the vector comprises a polynucleotide encoding a heavy chain complementarity determining region (HCDR) 1 of SEQ ID NOs: 103, 109, 115, 121, or 127.

In one embodiment, the vector comprises a polynucleotide encoding a HCDR2 of SEQ ID NOs: 104, 110, 116, 122, or 128.

In one embodiment, the vector comprises a polynucleotide encoding a HCDR3 of SEQ ID NOs: 105, 111, 117, 123, or 129.

In one embodiment, the vector comprises a polynucleotide encoding a HDCR1 of SEQ ID NOs: 103, 109, 115, 121, or 127; a HCDR2 of SEQ ID NOs: 104, 110, 116, 122, or 128; and a HCDR3 of SEQ ID NOs: 105, 111, 117, 123, or 129.

In one embodiment, the vector comprises a polynucleotide encoding a HCDR1, HCDR2 and HCDR3 of:
SEQ ID NOs: 103, 104, and 105, respectively;
SEQ ID NOs: 109, 110, and 111, respectively;
SEQ ID NOs: 115, 116, and 117, respectively;
SEQ ID NOs: 121, 122, and 123, respectively; or
SEQ ID NOs: 127, 128, and 129, respectively.

In one embodiment, the vector comprises a polynucleotide encoding a light chain complementarity determining region (LCDR) 1 of SEQ ID NOs: 106, 124, or 130.

In one embodiment, the vector comprises a polynucleotide encoding a LCDR2 of SEQ ID NOs: 107, 125, or 131.

In one embodiment, the vector comprises a polynucleotide encoding a LCDR3 of SEQ ID NOs: 108, 126, or 132.

In one embodiment, the vector comprises a polynucleotide encoding a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the vector comprises a polynucleotide encoding a LCDR1, LCDR2 and LCDR3 of:
SEQ ID NOs: 106, 107, and 108, respectively;
SEQ ID NOs: 124, 125, and 126, respectively; or
SEQ ID NOs: 130, 131, and 132, respectively.

In one embodiment, the vector comprises a polynucleotide encoding a HDCR1 of SEQ ID NOs: 103, 109, 115, 121, or 127; a HCDR2 of SEQ ID NOs: 104, 110, 116, 122, or 128; a HCDR3 of SEQ ID NOs: 105, 111, 117, 123, or 129; a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the vector comprises a polynucleotide encoding a HCDR1, HCDR2, HCDR3, LCDR1, LCDR2 and LCDR3 of:
SEQ ID NOs: 103, 104, 105, 106, 107, and 108, respectively;
SEQ ID NOs: 109, 110, 111, 106, 107, and 108, respectively;
SEQ ID NOs: 115, 116, 117, 106, 107, and 108, respectively;
SEQ ID NOs: 121, 122, 123, 124, 125, and 126, respectively; or
SEQ ID NOs: 127, 128, 129, 130, 131, and 132 respectively.

In one embodiment, the vector comprises a polynucleotide encoding the VH of a phosphorylated CRTC3 binding protein comprising a nucleotide sequence of SEQ ID NO: 185.

In one embodiment, the vector comprises a polynucleotide encoding the VL of a phosphorylated CRTC3 binding protein comprising a nucleotide sequence of SEQ ID NO: 186.

In one embodiment, the vector comprises a polynucleotide encoding a VH of SEQ ID NOs: 31, 65, 99, or 133.

In one embodiment, the vector comprises a polynucleotide encoding a VL of SEQ ID NOs: 32, 66, 100, or 134.

In one embodiment, the vector comprises a polynucleotide encoding a VH of SEQ ID NOs: 31, 65, 99, or 133; and the VL of SEQ ID NOs: 32, 66, 100, or 134.

In one embodiment, the vector comprises a polynucleotide encoding a VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

In one embodiment, the vector comprises a polynucleotide comprising a nucleotide sequence of ID NOs: 179, 181, 183, or 185; and a nucleotide sequence of SEQ ID NOs: 180, 182, 184, or 186.

In one embodiment, the vector comprises a polynucleotide encoding a phosphorylated CRTC3 binding protein comprising at least two nucleotide sequences of:
SEQ ID NOs: 179 and 180;
SEQ ID NOs: 181 and 182;
SEQ ID NOs: 183 and 184; or
SEQ ID NOs: 185 and 186.

In one embodiment, the vector comprises a polynucleotide encoding a heavy chain (HC) of SEQ ID NOs: 33, 67, 101, or 135.

In one embodiment, the vector comprises a polynucleotide encoding a light chain (LC) of SEQ ID NOs: 34, 68, 102, or 136.

In one embodiment, the vector comprises a polynucleotide encoding a HC of SEQ ID NOs: 33, 67, 101, or 135; and a LC of SEQ ID NOs: 34, 68, 102, or 136.

In one embodiment, the vector comprises a polynucleotide encoding the HC and LC of:
SEQ ID NOs: 33 and 34, respectively;
SEQ ID NOs: 67 and 68, respectively;
SEQ ID NOs: 101 and 102, respectively; or
SEQ ID NOs: 135 and 136, respectively.

Embodiments of the invention further provide host cells comprising any of the recombinant expression vectors described herein. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. In one embodiment, the host cell of the present invention comprises any cell line capable of expressing glycosylated mammalian proteins after transfection with a vector. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). The host cell may be a T cell.

Also provided are a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, an erythrocyte, a neutrophil, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

The disclosure also provides a method of producing the phosphorylated CRTC3 binding protein of the disclosure comprising culturing the host cell of the disclosure in conditions that the CRTC3 binding protein is expressed, and recovering the phosphorylated CRTC3 binding protein produced by the host cell. Methods of making proteins and purifying them are known. Once synthesized (either chemically or recombinantly), the phosphorylated CRTC3 binding proteins may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

The polynucleotides encoding the phosphorylated CRTC3 binding proteins of the disclosure may be incorporated into vectors using standard molecular biology methods. Host cell transformation, culture, antibody expression and purification are done using well known methods.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N6-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N6-isopentenyladenine, uracil-5-oxyacetic acid (v), wybutoxosine, pseudouracil, queuosine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

### Pharmaceutical Compositions/Administration

The disclosure also provides a pharmaceutical composition comprising the phosphorylated CRTC3 binding protein of the disclosure and a pharmaceutically acceptable carrier.

The disclosure also provides a pharmaceutical composition comprising the antigen binding domain that binds phosphorylated CRTC3 of the disclosure and a pharmaceutically acceptable carrier.

The disclosure also provides a pharmaceutical composition comprising the protein comprising the antigen binding domain that binds phosphorylated CRTC3 of the disclosure and a pharmaceutically acceptable carrier.

The disclosure also provides a pharmaceutical composition comprising the multispecific protein comprising the antigen binding domain that binds phosphorylated CRTC3 of the disclosure and a pharmaceutically acceptable carrier.

The disclosure also provides a pharmaceutical composition comprising the antigen binding domain that binds phosphorylated CRTC3 of the disclosure and a pharmaceutically acceptable carrier.

For therapeutic use, the phosphorylated CRTC3 binding protein of the disclosure may be prepared as pharmaceutical compositions containing an effective amount of the antibody as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the phosphorylated CRTC3 binding proteins of the invention in such pharmaceutical formulation may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration of the phosphorylated CRTC3 binding protein of the disclosure may be any suitable route such as parenteral administration, e.g., intradermal, intramuscular, intraperitoneal, intravenous or subcutaneous, transmucosal (oral, intranasal, intravaginal, rectal) or other means appreciated by the skilled artisan, as well known in the art.

The phosphorylated CRTC3 binding protein of the disclosure of the invention may also be administered prophylactically in order to reduce the risk of developing a disease such as cancer.

Thus, a pharmaceutical composition of the invention for intramuscular injection may be prepared to contain 1 ml sterile buffered water, and between about 1 ng to about 100 mg/kg, e.g. about 50 ng to about 30 mg/kg or more preferably, about 5 mg to about 25 mg/kg, of the phosphorylated CRTC3 binding protein of the disclosure of the invention.

In embodiments of the present disclosure, the phosphorylated CRTC3 binding protein-expressing cells may be provided in compositions, e.g., suitable pharmaceutical composition(s) comprising the phosphorylated CRTC3 binding protein-expressing cells and a pharmaceutically acceptable carrier. In one aspect, the present disclosure provides pharmaceutical compositions comprising an effective amount of a lymphocyte expressing one or more of the phosphorylated CRTC3 binding proteins described and a pharmaceutically acceptable excipient. Pharmaceutical compositions of the present disclosure may comprise a phosphorylated CRTC3 binding protein-expressing cell, e.g., a plurality of phosphorylated CRTC3 binding protein-expressing cells, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

A pharmaceutically acceptable carrier can include a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

### Methods of Detecting and Differentiating CRTC3

The disclosure also provides a method of detecting phosphorylated CRTC3 in a sample, comprising obtaining the sample, contacting the sample with the antigen binding domain that binds phosphorylated CRTC3 of the disclosure and detecting the bound phosphorylated CRTC3 in the sample.

The disclosure further provides a method of differentiating between phosphorylated and unphosphorylated CRTC3 in a sample, comprising obtaining the sample, contacting the sample with the antigen binding domain that binds phosphorylated CRTC3 of the disclosure, and detecting the presence of bound phosphorylated CRCT3, thereby determining the CRTC3 in the sample is phosphorylated.

In one embodiment, the disclosure further provides a method of differentiating between phosphorylated and unphosphorylated CRTC3 in a sample, comprising obtaining the sample, contacting the sample with the antigen binding domain that binds phosphorylated CRTC3 of the disclosure, and detecting the absence of bound phosphorylated CRCT3, thereby determining the CRTC3 in the sample is unphosphorylated.

The disclosure further provides a method of differentiating between phosphorylated and unphosphorylated CRTC3 in a sample, comprising obtaining the sample, contacting the sample with the antigen binding domain that binds phosphorylated CRTC3 of the disclosure, contacting the sample with an antigen binding domain (i.e. an antibody) that binds to total CRTC3 (i.e. dephosphorylated and phosphorylated CRTC3), detecting bound phosphorylated CRTC3 and bound total CRTC3 in the sample, and determining that said CRTC3 in the sample is phosphorylated.

In one embodiment, the disclosure further provides a method of differentiating between phosphorylated and unphosphorylated CRTC3 in a sample, comprising obtaining the sample, contacting the sample with the antigen binding domain that binds phosphorylated CRTC3 of the disclosure, contacting the sample with an antigen binding domain (i.e. an antibody) that binds to total CRTC3 (i.e. dephosphorylated and phosphorylated CRTC3), detecting bound total CRTC3 in the sample but not detecting bound phosphorylated CRTC3, and determining that said CRTC3 in the sample is not phosphorylated.

The disclosure further provides a method of differentiating between CRTC3 phosphorylated on serine 329 (pSer329), CRTC3 phosphorylated on serine 370 (pSer370), CRTC3 phosphorylated on serine 329 (pSer329) and serine 370 (pSer370), and CRTC3 not phosphorylated on serine 329 (pSer329) or serine 370 (pSer370), comprising obtaining the sample, contacting the sample with the antigen binding domain that binds CRTC3 pSer329 of the disclosure, contacting the sample with the antigen binding domain that binds CRTC3 pSer370 of the disclosure, not detecting bound CRTC3 pSer329 or bound CRTC3 pSer370, and determining that said CRTC3 in the sample is not phosphorylated on pSer329 or pSer370.

In one embodiment, the disclosure further provides a method of differentiating between CRTC3 phosphorylated on serine 329 (pSer329), CRTC3 phosphorylated on serine 370 (pSer370), CRTC3 phosphorylated on serine 329 (pSer329) and serine 370 (pSer370), and CRTC3 not phosphorylated on serine 329 (pSer329) or serine 370 (pSer370), comprising obtaining the sample, contacting the sample with the antigen binding domain that binds CRTC3 pSer329 of the disclosure, contacting the sample with the antigen binding domain that binds CRTC3 pSer370 of the disclosure, detecting bound CRTC3 pSer329 but not detecting bound CRTC3 pSer370, and determining that said CRTC3 in the sample is phosphorylated on pSer329.

In embodiment, the disclosure further provides a method of differentiating between CRTC3 phosphorylated on serine 329 (pSer329), CRTC3 phosphorylated on serine 370 (pSer370), CRTC3 phosphorylated on serine 329 (pSer329) and serine 370 (pSer370), and CRTC3 not phosphorylated on serine 329 (pSer329) or serine 370 (pSer370), comprising obtaining the sample, contacting the sample with the antigen binding domain that binds CRTC3 pSer329 of the disclosure, contacting the sample with the antigen binding domain that binds CRTC3 pSer370 of the disclosure, detecting bound CRTC3 pSer370 but not detecting bound CRTC3 pSer329, and determining that said CRTC3 in the sample is phosphorylated on pSer370.

The disclosure further provides a method of differentiating between CRTC3 phosphorylated on serine 329 (pSer329), CRTC3 phosphorylated on serine 370 (pSer370), CRTC3 phosphorylated on serine 329 (pSer329) and serine 370 (pSer370), and CRTC3 not phosphorylated on serine 329 (pSer329) or serine 370 (pSer370), comprising obtaining the sample, contacting the sample with the antigen binding domain that binds CRTC3 pSer329 of the disclosure, contacting the sample with the antigen binding domain that binds CRTC3 pSer370 of the disclosure, detecting bound CRTC3 pSer329 and detecting bound CRTC3 pSer370, and determining that said CRTC3 in the sample is phosphorylated on pSer329 and pSer370.

The disclosure further provides a method of detecting the proportion of phosphorylated CRTC3 in a sample, comprising obtaining the sample, contacting the sample with the antigen binding domain that binds phosphorylated CRTC3 of the disclosure, contacting the sample with an antigen binding domain (i.e. an antibody) that binds to total CRTC3 (i.e. dephosphorylated and phosphorylated CRTC3), detecting the bound phosphorylated CRTC3 in the sample, thereby determining the level of phosphorylated CRTC3, detecting the bound total CRTC3 in the sample, thereby determining the level of total CRTC3, and dividing the level of phosphorylated CRTC3 by the level of total CRTC3, thereby determining the proportion of phosphorylated CRTC3 in the sample. In some of any one of the above- or below-mentioned embodiments, the sample may be derived from urine, blood, serum, plasma, saliva, ascites, circulating cells, synovial fluid, circulating cells, cells that are not tissue associated (i.e., free cells), tissues (e.g., surgically resected tissue, biopsies, including fine needle aspiration), histological preparations, and the like.

The antigen binding domain that binds phosphorylated CRTC3 of the disclosure or the antibody that binds to total CRTC3 may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, 111In-DOTA, 111In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antigen binding domain that binds phosphorylated CRTC3 of the disclosure may be used in a variety of assays to detect phosphorylated CRTC3 in the sample. Exemplary assays are western blot analysis, radioimmunoassay, surface plasmon resonance, immunoprecipitation, equilibrium dialysis, immunodiffusion, electrochemiluminescence (ECL) immunoassay (including MSD™ assays), immunohistochemistry, fluorescence-activated cell sorting (FACS) or ELISA assay.

One of skill in the art will recognize that any antibody that binds to total CRTC3 (i.e. dephosphorylated and phosphorylated CRTC3), presently available or made available henceforth, can be used in conjunction with the antigen binding domain that binds phosphorylated CRTC3 of the disclosure in the methods of the present invention. Exemplary antibodies that bind to dephosphorylated and phosphorylated CRTC3 include, but are not limited to, rabbit monoclonal antibody EPR3440 (ABCAM™), rabbit monoclonal antibody C35G4 (CELL SIGNALING TECHNOLOGY™), rabbit polyclonal antibody NBP1-46832 (NOVUS BIOLOGICALS™), rabbit polyclonal antibody NBP1-83615 (NOVUS BIOLOGICALS™), rabbit polyclonal antibody NBP2-56127 (NOVUS BIOLOGICALS™), mouse monoclonal antibody 1D9 (United States Biological), mouse monoclonal antibody 2E0 (Creative Diagnostics), and mouse monoclonal antibody 5G9 (NOVUS BIOLOGICALS™) Methods of Screening

The disclosure also provides a method of screening one or more agents that modulates the phosphorylation state of CRTC3 proteins. For example, in one embodiment, the method comprises screening one or more agents that modulates the activity of one or more proteins that modulates the phosphorylation state of CRTC3 proteins. In one embodiment, the method comprises obtaining a composition, or one or more cells comprising CRTC3 and one or more protein that modulates the phosphorylation state of CRTC3, contacting the composition or the one or more cells with one or more agents, and detecting the phosphorylation state of CRTC3 according to any of the methods described in the present disclosure.

In one embodiment, detection of a decrease in phosphorylated CRTC3, relative to a comparator, identifies the agent as an inhibitor of CRTC3 phosphorylation. In one embodiment, detection of an increase in phosphorylated CRTC3, relative to a comparator, identifies the agent as an enhancer of CRTC3 phosphorylation.

In one embodiment, the agent modulates one or more proteins that modulate the phosphorylation state of CRTC3 proteins. In one embodiment, the one or more proteins comprises a serine/threonine kinase. In one embodiment, the serine/threonine kinase includes but is not limited to SIK1, SIK2 (QIK), SIK3 (QSK), AMPKa1, AMPKa2, MARK2 (Berdeaux and Hutchins, Frontiers in Endocrinology, 2019, 10:535) MAPK (e.g. ERK1, ERK2, etc.), and CDKs (e.g. CDK1, CDK2, CDK5, etc.). In one embodiment, the one or more proteins comprises a phosphatase. In one embodiment, the phosphatase comprises one or more selected from the group including, but not limited to, calcium-dependent calcineurin (CaN), PP1, and PP2A (Sonntag et al., iScience, 2019, 11:134-145). In one embodiment, the identified inhibitor of CRTC3 phosphorylation comprises a kinase inhibitor or a phosphatase enhancer. In one embodiment, the identified enhancer of CRTC3 phosphorylation comprises a kinase enhancer or a phosphatase inhibitor.

In one embodiment, the one or more cells comprises any cell known in the art to endogenously express CRTC3 and said one or more proteins that modulates the phosphorylation state of CRTC3. In one embodiment, the one or more cells can be any cell known in the art to be amenable to exogenous expression of CRTC3 and said one or more proteins that modulates the phosphorylation state of CRTC3.

In one embodiment, the agent can include, but should not be construed as being limited to, a small molecule, a chemical compound, a protein, a peptide, a peptidomimetic, an antibody (e.g. polyclonal or monoclonal), or a nucleic acid molecule. Additionally, an agent that modulates one or more proteins that modulates the phosphorylation state of CRTC3 encompasses a chemically modified compound, and derivatives, as is well known to one of skill in the chemical arts.

When the agent is a small molecule, chemical compound or peptidomimetic, it may be obtained using standard methods known to the skilled artisan. Such methods include chemical organic synthesis or biological means. Biological means include purification from a biological source, recombinant synthesis and in vitro translation systems, using methods well known in the art. Combinatorial libraries of molecularly diverse chemical compounds potentially useful in treating a variety of diseases and conditions are well known in the art as are methods of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

When the agent is a peptide, it may be synthesized using chemical synthesis technology well known in the art. Similarly, if the agent is a peptide, protein, or monoclonal antibody, a nucleic acid encoding the desired amino acid sequence may be cloned and expressed from an appropriate promoter sequence in cells suitable for the generation of large quantities of peptide, protein or monoclonal antibody. When the agent is a polyclonal antibody, the generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY). When the agent is a nucleic acid encoding one or more agent as described herein, methods of generating said nucleic acids and/or vectors comprising said nucleic acid are described herein and well-known in the art. In some of any one of the above- or below-mentioned embodiments, the agent is a nucleic acid capable of direct binding to CRTC3 and/or said one or more protein that modulates the phosphorylation state of CRTC3 (e.g. an aptamer).

### Methods of Isolation

The disclosure also provides a method of isolating phosphorylated CRTC3 from a sample, comprising obtaining the sample, contacting the sample with the antigen binding domain that binds phosphorylated CRTC3 of the disclosure, wherein said antigen binding domain is conjugated to a substrate, washing the sample to remove any unbound species, and removing the bound phosphorylated CRTC3 in the sample from the antigen binding domain that binds phosphorylated CRTC3 of the disclosure, thereby generating an isolated composition of phosphorylated CRTC3.

Certain embodiments of the invention may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polypeptides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polypeptides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers.

In some of any one of the above- or below-mentioned embodiments, the solid support comprises microspheres or beads. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Detection Guide” from Bangs Laboratories, Fishers Ind. is a helpful guide. In certain embodiments, the microspheres are magnetic microspheres or beads.

The beads need not be spherical; irregular particles may be used. Alternatively, or additionally, the beads may be porous. The bead sizes range from nanometers, i.e. 100 nm, to millimeters, i.e. 1 mm, with beads from about 0.2 micron to about 200 microns being preferred, and from about 0.5 to about 5 micron being particularly preferred, although in some of any one of the above- or below-mentioned embodiments smaller or larger beads may be used.

### Methods of Diagnosis

The present disclosure also provides a method of diagnosing or assessing the risk of developing one or more diseases or disorders associated with aberrant CRTC3 phosphorylation in a subject. In one embodiment, the method comprises diagnosing one or more diseases or disorders associated with elevated CRTC3 phosphorylation in a subject, the method comprising obtaining a sample from the subject, contacting the sample with the antigen binding domain that binds phosphorylated CRTC3 of the disclosure, detecting an elevated level of bound phosphorylated CRTC3 in the sample of the subject as compared to the level of bound phosphorylated CRTC3 of a comparator, and diagnosing said subject with one or more diseases or disorders associated with elevated phosphorylation of CRTC3.

In one embodiment, the method comprises diagnosing one or more diseases or disorders associated with depressed CRTC3 phosphorylation in a subject, the method comprising obtaining a sample from the subject, contacting the sample with the antigen binding domain that binds phosphorylated CRTC3 of the disclosure, detecting a depressed level of bound phosphorylated CRTC3 in the sample of the subject as compared to the level of bound phosphorylated CRTC3 of a comparator, and diagnosing said subject with one or more diseases or disorders associated with depressed phosphorylation of CRTC3.

In one embodiment, the method comprises assessing the risk of developing one or more diseases or disorders associated with elevated CRTC3 phosphorylation in a subject, the method comprising obtaining a sample from the subject, contacting the sample with the antigen binding domain that binds phosphorylated CRTC3 of the disclosure, detecting an elevated level of bound phosphorylated CRTC3 in the sample of the subject as compared to the level of bound phosphorylated CRTC3 of a comparator, and determining that said subject is at risk of developing one or more diseases or disorders associated with elevated phosphorylation of CRTC3.

In one embodiment, the method comprises assessing the risk of developing one or more diseases or disorders associated with depressed CRTC3 phosphorylation in a subject, the method comprising obtaining a sample from the subject, contacting the sample with the antigen binding domain that binds phosphorylated CRTC3 of the disclosure, detecting a depressed level of bound phosphorylated CRTC3 in the sample of the subject as compared to the level of bound phosphorylated CRTC3 of a comparator, and determining that said subject is at risk of developing one or more diseases or disorders associated with depressed phosphorylation of CRTC3.

In one embodiment, the one or more diseases or disorders associated with aberrant CRTC3 phosphorylation includes, but is not limited to, Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease or an ulcerative colitis. In one embodiment, the one or more diseases or disorders associated with aberrant CRTC3 phosphorylation includes, but is not limited to, an ankylosing spondylitis, an atherosclerosis, an autoimmune diabetes, a primary biliary cholangitis, an inflammatory bowel disease (IBD), a systemic lupus erythematosus, a multiple sclerosis, a psoriasis, an arthritis, Sjogren's syndrome, a transplantation, a graft versus host disease (GVHD), a type I diabetes, a rheumatoid arthritis, a juvenile arthritis, a psoriatic arthritis, Celiac Disease, Crohn's disease or an ulcerative colitis.

Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level of phosphorylated CRTC3 in a sample obtained from a subject not having a disease or disorder. In one embodiment, the comparator is a level of phosphorylated CRTC3 in a sample obtained from a subject known not to have a disease or disorder.

In various embodiments of the methods of the invention, the level of phosphorylated CRTC3 is determined to be increased when the level of phosphorylated CRTC3 in the sample is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level of phosphorylated CRTC3 is determined to be increased when the level of phosphorylated CRTC3 in the sample is increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In various embodiments of the methods of the invention, the level of phosphorylated CRTC3 is determined to be decreased when the level of phosphorylated CRTC3 in the sample is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level of phosphorylated CRTC3 is determined to be decreased when the level of phosphorylated CRTC3 in the sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

### Methods of Treatment and Uses

The antigen binding domain that binds phosphorylated CRTC3 of the disclosure may be administered to a subject in need thereof to manage, treat, prevent, or ameliorate a disease or disorder or one or more symptoms thereof.

The disclosure also provides methods comprising administering a therapeutically effective amount of the antigen binding domain that binds phosphorylated CRTC3 of the disclosure to a subject having a disease or disorder.

The disclosure also provides methods comprising administering a therapeutically effective amount of the protein comprising the antigen binding domain that binds phosphorylated CRTC3 of the disclosure to a subject having a disease or disorder.

The disclosure also provides a method comprising administering a therapeutically effective amount of the multispecific protein comprising the antigen binding domain that binds phosphorylated CRTC3 of the disclosure to a subject having a disease or disease or disorder.

The disclosure also provides a method comprising administering a therapeutically effective amount of the pharmaceutical composition comprising the antigen binding domain that binds phosphorylated CRTC3 of the disclosure to a subject having a disease or disorder.

The disclosure also provides methods of treating a disease or disorder in a subject comprising administering a therapeutically effective amount of the antigen binding domain that binds phosphorylated CRTC3 of the disclosure to the subject in need thereof for a time sufficient to treat the disease or disorder.

The disclosure also provides methods of treating a disease or disorder in a subject comprising administering a therapeutically effective amount of the protein comprising the antigen binding domain that binds phosphorylated CRTC3 of the disclosure to the subject for a time sufficient to treat the disease or disorder.

The disclosure also provides a method of treating a disease or disorder in a subject, comprising administering a therapeutically effective amount of the multispecific protein comprising the antigen biding domain that binds phosphorylated CRTC3 of the disclosure to the subject for a time sufficient to treat the disease or disorder.

The disclosure also provides a method of treating a disease or disorder in a subject, comprising administering a therapeutically effective amount of the pharmaceutical composition comprising the antigen binding domain that binds phosphorylated CRTC3 of the disclosure to the subject for a time sufficient to treat the disease or disorder.

The disclosure also provides methods of preventing a disease or disorder in a subject comprising administering a therapeutically effective amount of the antigen binding domain that binds phosphorylated CRTC3 of the disclosure to the subject in need thereof for a time sufficient to prevent the disease or disorder. In certain embodiments, preventing comprises treating an asymptomatic subject. In certain embodiments, preventing comprises preventing the onset of disease symptoms in a subject.

The disclosure also provides methods of preventing a disease or disorder in a subject comprising administering a therapeutically effective amount of the protein comprising the antigen binding domain that binds phosphorylated CRTC3 of the disclosure to the subject for a time sufficient to prevent the disease or disorder.

The disclosure also provides a method of preventing a disease or disorder in a subject, comprising administering a therapeutically effective amount of the multispecific protein comprising the antigen binding domain that binds phosphorylated CRTC3 of the disclosure to the subject for a time sufficient to prevent the disease or disorder.

The disclosure also provides a method of preventing a disease or disorder in a subject, comprising administering a therapeutically effective amount of the pharmaceutical composition comprising the antigen binding domain that binds phosphorylated CRTC3 of the disclosure to the subject for a time sufficient to prevent the disease or disorder.

In one embodiment, the one or more disease or disorder includes, but is not limited to, Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease or an ulcerative colitis. In one embodiment, the one or more disease or disorder, includes but is not limited to, an ankylosing spondylitis, an atherosclerosis, an autoimmune diabetes, a primary biliary cholangitis, an inflammatory bowel disease (IBD), a systemic lupus erythematosus, a multiple sclerosis, a psoriasis, an arthritis, Sjogren's syndrome, a transplantation, a graft versus host disease (GVHD), a type I diabetes, a rheumatoid arthritis, a juvenile arthritis, a psoriatic arthritis, Celiac Disease, Crohn's disease or an ulcerative colitis.

The disclosure also provides methods comprising administering a therapeutically effective amount of a composition comprising an antigen binding domain that binds phosphorylated CRTC3 of the disclosure to the subject in need thereof, wherein the antigen binding domain that binds phosphorylated CRTC3 comprises (a) a HDCR1 of SEQ ID NOs: 1, 7, 13, 19 or 25; a HCDR2 of SEQ ID NOs: 2, 8, 14, 20 or 26; a HCDR3 of SEQ ID NOs: 3, 9, 15, 21 or 27; a LDCR1 of SEQ ID NOs: 4, 22, or 28; a LCDR2 of SEQ ID NOs: 5, 23, or 29; and a LCDR3 of SEQ ID NOs: 6, 24, or 30; (b) a HDCR1 of SEQ ID NOs: 35, 41, 47, 53 or 59; a HCDR2 of SEQ ID NOs: 36, 42, 48, 54 or 60; a HCDR3 of SEQ ID NOs: 37, 43, 49, 55 or 61; a LDCR1 of SEQ ID NOs: 38, 56, or 62; a LCDR2 of SEQ ID NOs: 39, 57, or 63; and a LCDR3 of SEQ ID NOs: 40, 58, or 64; (c) a HDCR1 of SEQ ID NOs: 69, 75, 81, 87, or 93; a HCDR2 of SEQ ID NOs: 70, 76, 82, 88, or 94; a HCDR3 of SEQ ID NOs: 71, 77, 83, 89, or 95; a LDCR1 of SEQ ID NOs: 72, 90, or 96; a LCDR2 of SEQ ID NOs: 73, 91, or 97; and a LCDR3 of SEQ ID NOs: 74, 92, or 98; or (d) a HDCR1 of SEQ ID NOs: 103, 109, 115, 121, or 127; a HCDR2 of SEQ ID NOs: 104, 110, 116, 122, or 128; a HCDR3 of SEQ ID NOs: 105, 111, 117, 123, or 129; a LDCR1 of SEQ ID NOs: 106, 124, or 130; a LCDR2 of SEQ ID NOs: 107, 125, or 131; and a LCDR3 of SEQ ID NOs: 108, 126, or 132.

In one embodiment, the antigen binding domain that binds phosphorylated CRTC3 comprises a VH of SEQ ID NOs: 31, 65, 99, or 133; and the VL of SEQ ID NOs: 32, 66, 100, or 134. In one embodiment, the antigen binding domain that binds phosphorylated CRTC3 comprises a VH and VL of:
SEQ ID NOs: 31 and 32, respectively;
SEQ ID NOs: 65 and 66, respectively;
SEQ ID NOs: 99 and 100, respectively; or
SEQ ID NOs: 133 and 134, respectively.

The disclosure also provides methods of determining the efficacy of one or more therapeutics that modulates the level of phosphorylation of CRTC3 in a subject. In one embodiment, the method comprises determining the efficacy of one or more therapeutics that elevates the level of phosphorylation of CRTC3 in a subject, comprising administering to the subject the one or more therapeutics, obtaining a sample from the subject, contacting the sample with the antigen binding domain that binds phosphorylated CRTC3 of the disclosure, detecting an elevated level of bound phosphorylated CRTC3 in the sample of the subject as compared to the level of bound phosphorylated CRTC3 of a comparator, and determining that said one or more therapeutics is efficacious for elevating the level of phosphorylated CTRC3 in a subject.

In one embodiment, the method comprises determining the efficacy of one or more therapeutics that depresses the level of phosphorylation of CRTC3 in a subject, comprising administering to the subject the one or more therapeutics, obtaining a sample from the subject, contacting the sample with the antigen binding domain that binds phosphorylated CRTC3 of the disclosure, detecting a depressed level of bound phosphorylated CRTC3 in the sample of the subject as compared to the level of bound phosphorylated CRTC3 of a comparator, and determining that said one or more therapeutics is efficacious for depressing the level of phosphorylated CTRC3 in a subject. In one embodiment, the one or more therapeutics that depresses the level of phosphorylation of CRTC3 in a subject is a Salt-Inducible Kinase (SIK) inhibitor.

In one embodiment, the method comprises determining the efficacy of one or more therapeutics that elevates the level of phosphorylation of CRTC3 in a subject, comprising administering to the subject the one or more therapeutics, obtaining a sample from the subject, contacting the sample with the antigen binding domain that binds phosphorylated CRTC3 of the disclosure, not detecting an elevated level of bound phosphorylated CRTC3 in the sample of the subject as compared to the level of bound phosphorylated CRTC3 of a comparator, thereby determining that said one or more therapeutics is not efficacious for elevating the level of phosphorylated CTRC3 in the subject. In one embodiment, the method comprises administering one or more additional therapeutics that elevate the level of phosphorylation of CRTC3 in the subject. In one embodiment, said administering of one or more additional therapeutics that elevate the level of phosphorylation of CRTC3 in a subject is repeated as necessary until an elevated level of bound phosphorylated CRTC3 in the sample of the subject as compared to the level of bound phosphorylated CRTC3 of a comparator is detected.

In one embodiment, the method comprises determining the efficacy of one or more therapeutics that depresses the level of phosphorylation of CRTC3 in a subject, comprising administering to the subject the one or more therapeutics, obtaining a sample from the subject, contacting the sample with the antigen binding domain that binds phosphorylated CRTC3 of the disclosure, not detecting a depressed level of bound phosphorylated CRTC3 in the sample of the subject as compared to the level of bound phosphorylated CRTC3 of a comparator, thereby determining that said one or more therapeutics is not efficacious for depressing the level of phosphorylated CTRC3 in a subject. In one embodiment, the method comprises administering one or more additional therapeutics that depress the level phosphorylation of CRTC3 in a subject. In one embodiment, the one or more therapeutics that depresses the level of phosphorylation of CRTC3 in a subject is a Salt-Inducible Kinase (SIK) inhibitor. In one embodiment, said administering of one or more additional therapeutics that depress the level of phosphorylation of CRTC3 in a subject is repeated as necessary until a depressed level of bound phosphorylated CRTC3 in the sample of the subject as compared to the level of bound phosphorylated CRTC3 of a comparator is detected.

The disclosure also provides methods of determining a therapeutically effective dose for one or more therapeutics that modulates the level of phosphorylation of CRTC3 in a subject. In one embodiment, the method comprises determining a therapeutically effective dose for one or more therapeutics that elevates the level of phosphorylation of CRTC3 in a subject, comprising administering to the subject the one or more therapeutics, obtaining a sample from the subject, contacting the sample with the antigen binding domain that binds phosphorylated CRTC3 of the disclosure, detecting an elevated level of bound phosphorylated CRTC3 in the sample of the subject as compared to the level of bound phosphorylated CRTC3 of a comparator, and determining that said one or more therapeutics was administered in a therapeutically effective amount to elevate the level of phosphorylated CTRC3 in a subject.

In one embodiment, the method comprises determining a therapeutically effective dose for one or more therapeutics that depresses the level of phosphorylation of CRTC3 in a subject, comprising administering to the subject the one or more therapeutics, obtaining a sample from the subject, contacting the sample with the antigen binding domain that binds phosphorylated CRTC3 of the disclosure, detecting a depressed level of bound phosphorylated CRTC3 in the sample of the subject as compared to the level of bound phosphorylated CRTC3 of a comparator, and determining that said one or more therapeutics was administered in a therapeutically effective amount to depress the level of phosphorylated CTRC3 in a subject.

In one embodiment, the method comprises determining a therapeutically effective dose for one or more therapeutics that elevates the level of phosphorylation of CRTC3 in a subject, comprising administering to the subject the one or more therapeutics, obtaining a sample from the subject, contacting the sample with the antigen binding domain that binds phosphorylated CRTC3 of the disclosure, not detecting an elevated level of bound phosphorylated CRTC3 in the sample of the subject as compared to the level of bound phosphorylated CRTC3 of a comparator, thereby determining that said one or more therapeutics was not administered in a therapeutically effective amount to elevate the level of phosphorylated CTRC3 in the subject. In one embodiment, the method comprises administering to the subject an increased concentration of said one or more therapeutics that elevates the level of phosphorylation of CRTC3. In one embodiment, said administering to the subject of an increased concentration of said one or more therapeutics that elevates the level of phosphorylation of CRTC3 is repeated until an elevated level of bound phosphorylated CRTC3 in the sample of the subject as compared to the level of bound phosphorylated CRTC3 of a comparator is detected.

In one embodiment, the method comprises determining a therapeutically effective dose for one or more therapeutics that depresses the level of phosphorylation of CRTC3 in a subject, comprising administering to the subject the one or more therapeutics, obtaining a sample from the subject, contacting the sample with the antigen binding domain that binds phosphorylated CRTC3 of the disclosure, not detecting a depressed level of bound phosphorylated CRTC3 in the sample of the subject as compared to the level of bound phosphorylated CRTC3 of a comparator, thereby determining that said one or more therapeutics was not administered in a therapeutically effective amount to depress the level of phosphorylated CTRC3 in a subject. In one embodiment, the method comprises administering to the subject an increased concentration of said one or more therapeutics that depresses the level of phosphorylation of CRTC3. In one embodiment, said administering to the subject of an increased concentration of said one or more therapeutics that depresses the level of phosphorylation of CRTC3 is repeated until a depressed level of bound phosphorylated CRTC3 in the sample of the subject as compared to the level of bound phosphorylated CRTC3 of a comparator is detected.

In one embodiment, the comparator is derived from the subject prior to being administered the one or more therapeutics. In one embodiment, the comparator is derived from a separate subject (or subjects) that has not been administered the one or more therapeutics. In one embodiment, the comparator is derived from a subject (or subjects) that has been successfully treated with one or more therapeutics that elevates or depresses the level of phosphorylation of CRTC3.

In one embodiment, the therapeutic can include, but should not be construed as being limited to, a small molecule, a chemical compound, a protein, a peptide, a peptidomemetic, an antibody, or a nucleic acid molecule, as described herein.

When a therapeutically effective amount is indicated, the precise amount of the phosphorylated CRTC3-binding proteins or therapeutics that modulate the phosphorylation of CRTC3 of the present disclosure to be administered can be determined by a physician with consideration of individual differences in age, weight, and condition of the subject.

Delivery systems useful in the context of the phosphorylated CRTC3-binding proteins of the invention may include time-released, delayed release, and sustained release delivery systems such that the delivery of the phosphorylated CRTC3-binding protein compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

The administration of the phosphorylated CRTC3-binding proteins and compositions may be carried out in any manner, e.g., by parenteral or nonparenteral administration, including by aerosol inhalation, injection, infusions, ingestion, transfusion, implantation or transplantation. For example, the phosphorylated CRTC3-binding proteins and compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection. The compositions of phosphorylated CRTC3-binding proteins may be injected, for instance, directly into a tumor, lymph node, tissue, organ, or site of infection.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

### Combination Therapies

The phosphorylated CRTC3 binding proteins of the disclosure may be administered in combination with at least one additional therapeutics.

The phosphorylated CRTC3 binding proteins of the disclosure may also be administered in combination with one or more other therapies. In some of any one of the above- or below-mentioned embodiments, the phosphorylated CRTC3 binding proteins of the disclosure may be administered in combination with one or more other therapies useful for the prevention, management, treatment or amelioration of a disease or disorder or one or more symptoms thereof to a subject in need thereof to prevent, manage, treat or ameliorate a disease or disorder or one or more symptoms thereof.

In some of any one of the above- or below-mentioned embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some of any one of the above- or below-mentioned embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one embodiment, other therapeutic agents such as factors may be administered before, after, or at the same time (simultaneous with) as the phosphorylated CRTC3 binding proteins.

The phosphorylated CRTC3 binding proteins and the at least one additional therapeutic agent can be administered simultaneously, in the same or in separate compositions, or sequentially. For sequential administration, the phosphorylated CRTC3 binding proteins described herein can be administered first, and the additional agent can be administered second, or the order of administration can be reversed.

In one embodiment, the subject can be administered an agent which enhances the activity of a phosphorylated CRTC3 binding protein. For example, in one embodiment, the agent can be an agent which inhibits an inhibitory molecule.

The following examples are provided to further describe some of the embodiments disclosed herein. The examples are intended to illustrate, not to limit, the disclosed embodiments.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Monoclonal Antibody Generation

Specific detection of phosphorylated CRTC3 protein has been accomplished by immunoblotting using polyclonal sera from sheep (Clark K, et al., PNAS USA, 2012, 109(42): 16986-91; Hutchinson L D, et al., Cell death & disease, 2020, 11(1): 49; Ozanne J, et al., The Biochemical journal, 2015, 465(2): 271-9) or rabbits (Sonntag T, et al., PloS one, 2017, 12(2): e0173013) providing evidence for the utility of the assays described herein. However, the use of such polyclonal sera is limited by assay reliability over time due to limitations in reagent quantities of a given batch and the inherent variability between different batches produced in different animals at different times. Polyclonal sera also have the potential for cross-reactivity to non-desired protein species due to the recognition of multiple epitopes. The use of monoclonal antibodies (mAbs) mitigates these challenges because of their homogenous nature, high specificity, and the ability to produce them in unlimited amounts on an industrial scale at the same level of quality (Nelson P N, et al., Molecular pathology, 2000, 53(3): 111-7).

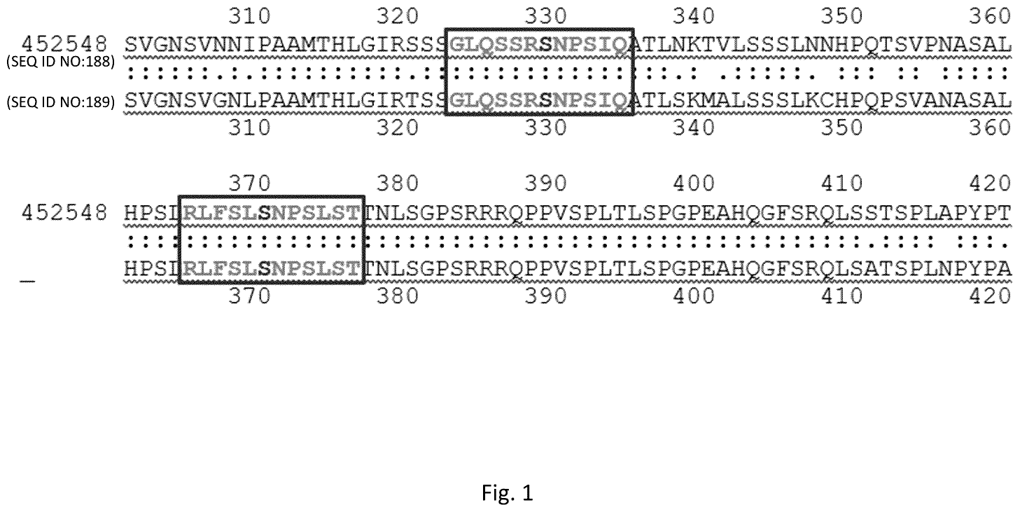

In the present experiments, mAbs were raised against specific portions of the CRTC3 protein, i.e. protein fragments that included phosphorylated serine residues 329 or 370 (FIG. 1). These protein fragments are conserved between, but not limited to, human, mice (FIG. 1), cynomolgus monkey, rhesus monkey, and rat (see uniprot, gene=CRTC3).

Peptide synthesis, rabbit immunizations, and polyclonal antibody generation was done by Epitomics/Abcam. Briefly, 12 amino acid peptides GLQSSRSNPSIQ (SEQ ID NO: 142) and RLFSLSNPSLST (SEQ ID NO: 144) were synthesized and the serine residues phosphorylated to yield peptides GLQSSRpSNPSIQ (pSer329; SEQ ID NO: 143) and RLFSLpSNPSLST (pSer370; SEQ ID NO: 145). For immunizations, the peptides were conjugated to Keyhole Limpet Hemocyanin (KLH) or Ovalbumin (OVA). Cohorts of four rabbits were immunized with either pSer329-KLH (three times) and pSer329-OVA (once) or pSer370-KLH (three times) and pSer370-OVA (once) during a seven-week period. After two additional weeks, the animals were boosted once with pSer329-OVA. Select animals received a third round of immunization with the KLH peptide conjugates 16 weeks after the first injection. Serum samples from each animal was tested for the apparent presence of antibodies reactive against the immunizing peptides before immunization, after 8 weeks, after ˜ 11 weeks, and after −18 weeks (where applicable) by ELISA using bovine serum albumin (BSA)-conjugated phosphorylated and unphoshorylated versions of the peptide. The sera were further tested for the apparent presence of pSer329 peptide- and pSer370 peptide-specific antibodies by immunoblotting using lysates from THP-1 cells (ATCC) that had either been treated with the adenylyl cyclase activator Forskolin (Sigma-Aldrich) to induce cAMP-dependent CRTC3 de-phosphorylation (Sonntag T, et al., PloS one, 2017, 12(2): e0173013) or left untreated (data not shown; see below for description of cell culture and immunoblotting procedure).

### Example 2: Monoclonal Antibody Characterization

Polyclonal antibodies were purified by affinity purification of antisera using each phosphorylated peptide for enrichment and each unphosporylated peptide for depletion. Presence of specific antibody was verified by analysis of the purified samples by ELISA and immunoblotting (as above; data not shown).

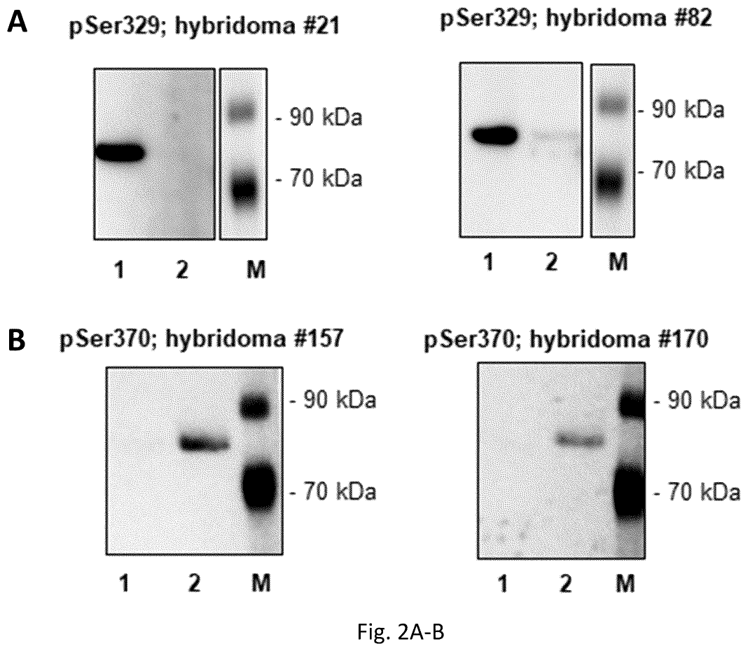

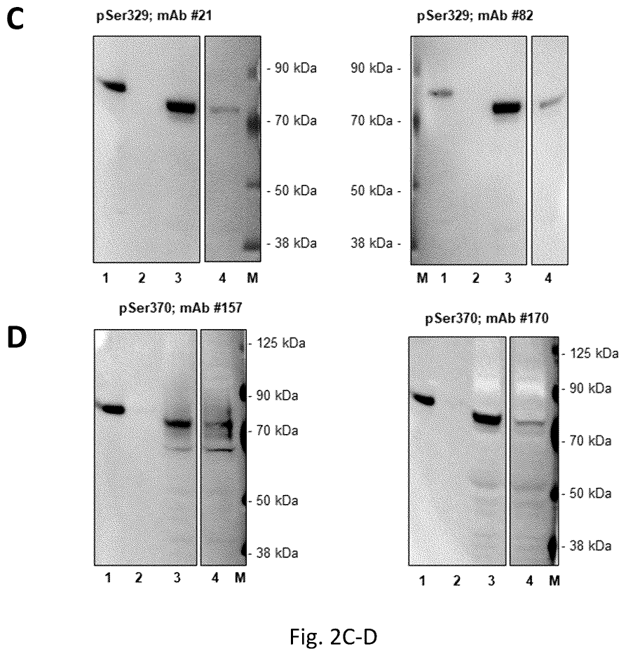

Animals that had generated antibodies with apparent specificity or selectivity to the phosphorylated peptides but not to the unphosphorylated peptides were sacrificed after −25 weeks to harvest spleens followed by lymphocyte isolation. Hybridomas were generated and characterized by ELISA and immunoblotting (FIG. 2A and FIG. 2B; see below for method description). Selected hybridomas were subjected to recombinant cloning and sequencing. The antibody heavy and light chain complementary DNA (cDNA) and amino acid sequences obtained for anti-phoshpo-Serine370 mAb #157 and #170 and anti-phospho-Serine329 mAb #21 and #82 are depicted in Table 2. Antibody protein was produced recombinantly in a mammalian cellular expression system, subjected to Protein A purification, and characterized by ELISA (as above) and immunoblotting (FIG. 2C and FIG. 2D; see below for method description).

To facilitate the identification of hybridomas that produced antibodies specific for the phosphorylated immunizing peptides, CRTC3 proteins were designed that harbored serine to alanine mutations at either position 329 (S329A mutant) or 370 (S370A mutant; Table 3) rendering them unable to carry phosphate groups at positions 329 and 370, respectively. In addition, CRTC3 protein harboring the unmutated wild type (WT) amino acid sequence was produced as a control reagent (Table 3). Briefly, proteins were overexpressed in a Baculovirus expression system and purified using M2 anti-flag magnetic beads (Millipore-Sigma). The wild type protein was additionally subjected to subsequent size exclusion chromatography. All purified proteins were phosphorylated in vitro in the presence of internally produced SIK2 variant (6His-MBP-TEV-SIK2-PKD-UBA; SEQ ID NO: 187) and 1 mM ATP (Promega) in a buffer containing 50 mM Tris pH 7.5, 100 mM NaCl, 10 mM MgCl2 (all from Quality Biologicals), 0.1 mM EDTA (Corning), 1 mM DTT (MP Biomedicals). Phosphorylation reactions were quenched by the addition of excess EDTA.

Immunoblotting was done using the NuPAGE electrophoresis and western Blotting system (Invitrogen). Briefly, 0.1-0.15 microgram of WT or S329A mutant or S370A mutant recombinant protein along with WesternSure® Pre-stained Chemiluminescent Protein Ladder (Li-Cor) were loaded onto NuPAGE® Bis-Tris gels and separated by electrophoresis. Subsequently, proteins were transferred onto nitrocellulose membranes (iBlot® 2 Transfer Stacks, nitrocellulose, mini; ThermoFisher) using an iBlot 2® Gel Transfer Device (ThermoFisher). Specific bands on the nitrocellulose membranes were detected using the automatic Western blot-processing iBind™ Flex Western System (ThermoFisher) as per the manufacturer's recommendations. As primary antibodies served either undiluted hybridoma supernatant or recombinantly expressed mAbs at 0.25 microgram/ml, whereas Peroxidase AffiniPure Donkey Anti-Rabbit IgG (H+L) (Jackson ImmunoResearch) was used as secondary antibody at a 1:4000 dilution. The blots were developed using Amersham ECL Western Blotting Detection Reagent and an ImageQuant LAS4000 image scanner (both GE Healthcare Life Sciences).

Tissue culture supernatant from hybridomas #21 and #82 (FIG. 2A) or recombinantly expressed mAbs #21 and #82 (FIG. 2C) specifically recognized only recombinant CRTC3 protein harboring the WT sequence (lane 1) but not the CRTC3 S329A variant (lane 2), as expected. Likewise, hybridomas #157 and #170 (FIG. 2B) or recombinant mAbs #157 and #170 (FIG. 2D) gave a specific signal only with WT (lane 2) but not S370A mutant CRTC3 protein (lane 1).

To test whether the recombinantly generated mAbs specifically recognize only phosphorylated CRTC3 in a complex cellular lysate after chemically induced CRTC3 dephosphorylation, THP1 acute monocytic leukemia cells were differentiated into macrophage-like cells (Tsuchiya S, et al., Cancer Res, 1982, 42(4): 1530-6) using 100 ng/ml phorbol 12-myristate 13-acetate (PMA) for 2 days followed by a 3 hour-incubation with 100 ng/ml lipopolysaccharide (LPS-EB Ultrapure; Invivogen). Cells were then either left untreated or incubated with dasatinib at 10 micromolar for 1.5 hours before cell harvest. Bcr-Abl/Src-inhibitor dasatinib was shown to also strongly inhibit the SIKs and induce dephosphorylation of CRTC3 (Ozanne J, et al., The Biochemical journal, 2015, 465(2): 271-9). Cells were washed in cold PBS (Gibco) and lysed on ice in 50 mM Tris HCl (Alfa Aesar) at pH 7.4, 1 mM DTT, 1% (vol/vol) Triton X-100 (both ThermoFisher), 1 mM EDTA (Gibco), 1 mM EGTA (Alfa Aesar) supplemented with protease inhibitor (cOmplete™ ULTRA Tablets; Roche) and phosphatase inhibitor (Phosstop, Roche) as per the manufacturer's recommendations for 1 hour. Extracted proteins were harvested by collecting the supernatant after centrifugation of the lysate for 20 minutes. Protein concentrations were determined using the Pierce Detergent-compatible Bradford Assay kit (Pierce). Lysate proteins were separated by gel electrophoresis and detected by immunoblotting as described above. As shown in FIG. 2C and FIG. 2D, recombinant mAbs #21, #82, #157, and #170 each produced a strong signal corresponding to proteins of the expected molecular size in THP1 lysates that had not been incubated with a SIK inhibitor (lane 3) whereas this signal was strongly diminished in THP1 lysates treated for 1.5 hours with the SIK inhibitor dasatinib (lane 4). That the detected signals indeed corresponded to CRTC3 was verified in independent experiments, in which similarly treated THP1 lysates were developed using an antibody recognizing CRTC3 independent of its phosphorylation status (EPR3440; purified; rabbit; Abcam; data not shown).

Thus, the present experiments demonstrate that mAbs #21 and #82 specifically detected human CRTC3 only when phosphorylated at serine 329 whereas mAbs #157 and #170 specifically recognized human CRTC3 only when phosphorylated at serine 370.

### Example 3: CRTC3 Assay Using Phospho-Specific Antibodies

An assay was developed to enable quantification of pCRTC3 levels, in which a pair of customized electrochemiluminescence-based sandwich immunoassays were developed using the Mesoscale Discovery (MSD®) platform. One assay utilized mAb #82 to selectively capture CRTC3 protein that carried a phosphate moiety at the Ser329 position whereas another utilized a commercially available antibody to capture total CRTC3 protein, i.e. independent of its phosphorylation state.

TABLE 2
HC and LC sequences, HC and LC variable regions sequences, as
well as CDRs (ABM definitions).
Nucleic acid sequences are denoted by *.
mAb#157

| | | SEQ ID |
|---|---|---|
| Region | Amino (or Nucleic*) Acid Sequence | NO: |
| HC AbM | GISLSSYGLS | 1 |
| CDR1 | | |
| HC AbM | AIGASGSAY | 2 |
| CDR2 | | |
| HC AbM | NYI | 3 |
| CDR3 | | |
| LC AbM | QASQSVYSDNRLS | 4 |
| CDR1 | | |
| LC AbM | EASKLAS | 5 |
| CDR2 | | |
| LC AbM | QGSYDCNSADCSA | 6 |
| CDR3 | | |
| HC Kabat | SYGLS | 7 |
| CDR1 | | |
| HC Kabat | AIGASGSAYYASWAKS | 8 |
| CDR2 | | |
| HC Kabat | NYI | 9 |
| CDR3 | | |
| LC Kabat | QASQSVYSDNRLS | 10 |
| CDR1 | | |
| LC Kabat | EASKLAS | 11 |
| CDR2 | | |
| LC Kabat | QGSYDCNSADCSA | 12 |
| CDR3 | | |
| HC Chothia | GISLSSY | 13 |
| CDR1 | | |
| HC Chothia | GASGS | 14 |
| CDR2 | | |
| HC Chothia | NYI | 15 |
| CDR3 | | |
| LC Chothia | QASQSVYSDNRLS | 16 |
| CDR1 | | |
| LC Chothia | EASKLAS | 17 |
| CDR2 | | |
| LC Chothia | QGSYDCNSADCSA | 18 |
| CDR3 | | |
| HC IMGT | GISLSSYG | 19 |
| CDR1 | | |
| HC IMGT | IGASGSA | 20 |
| CDR2 | | |
| HC IMGT | KRNYI | 21 |
| CDR3 | | |
| LC IMGT | QSVYSDNR | 22 |
| CDR1 | | |
| LC IMGT | EAS | 23 |
| CDR2 | | |
| LC IMGT | QGSYDCNSADCSA | 24 |
| CDR3 | | |
| HC | SSYGLS | 25 |
| CONTACT | | |
| CDR1 | | |
| HC | WIGAIGASGSAY | 26 |
| CONTACT | | |
| CDR2 | | |
| HC | KRNY | 27 |
| CONTACT | | |
| CDR3 | | |
| LC | YSDNRLSWY | 28 |
| CONTACT | | |
| CDR1 | | |
| LC | LLIYEASKLA | 29 |
| CONTACT | | |
| CDR2 | | |
| LC | QGSYDCNSADCS | 30 |
| CONTACT | | |
| CDR3 | | |
| VH | QEQLKESGGGLFKPMDILTVTCTVSGISLSSYGLSWVR | 31 |
| | QAPGNGLEWIGAIGASGSAYYASWAKSRSTITRNTNLN | |
| | TVTLKMTSLTAADTATYFCKRNYIWGPGTLVTVSS | |
| VL | AQVLTQTASPVSAAVGGTVTINCQASQSVYSDNRLSW | 32 |
| | YQQKPGQPPKLLIYEASKLASGVPPRFSGSGSGTQFTL | |
| | AISGVQCDDAATYYCQGSYDCNSADCSAFGGGTEVVV | |
| | K | |
| HC | METGLRWLLLVAVLKGVQCQEQLKESGGGLFKPMDILT | 33 |
| | VTCTVSGISLSSYGLSWVRQAPGNGLEWIGAIGASGSAY | |
| | YASWAKSRSTITRNTNLNTVTLKMTSLTAADTATYFCK | |
| | RNYIWGPGTLVTVSS | |
| LC | MDTRAPTQLLGLLLLWLPGATFAQVLTQTASPVSAAVG | 34 |
| | GTVTINCQASQSVYSDNRLSWYQQKPGQPPKLLIYEASK | |
| | LASGVPPRFSGSGSGTQFTLAISGVQCDDAATYYCQGSY | |
| | DCNSADCSAFGGGTEVVVK | |
| VH* | CAGGAGCAGTTGAAGGAATCCGGGGGAGGTCTCTTCA | 179 |
| | AGCCAATGGATATCCTGACAGTCACCTGCACAGTCTC | |
| | TGGAATCTCCCTCAGTAGCTATGGACTGAGCTGGGTC | |
| | CGCCAGGCTCCAGGGAACGGGCTGGAATGGATCGGA | |
| | GCCATTGGTGCTAGTGGTAGCGCATACTACGCGAGCT | |
| | GGGCGAAAAGCCGATCCACCATCACCAGAAACACCA | |
| | ACCTGAACACGGTGACTCTGAAAATGACCAGTCTGAC | |
| | AGCCGCGGACACGGCCACCTATTTCTGTAAAAGAAAT | |
| | TACATCTGGGGCCCAGGCACCCTGGTCACCGTCTCCT | |
| | CA | |
| VL* | GCTCAAGTGCTGACCCAGACTGCATCGCCCGTGTCTG | 180 |
| | CGGCTGTGGGAGGCACAGTCACCATCAACTGCCAGGC | |
| | CAGTCAGAGTGTTTATAGTGACAACCGCTTATCCTGG | |
| | TATCAACAGAAACCAGGGCAGCCTCCCAAGCTCCTGA | |
| | TCTACGAAGCATCCAAACTGGCATCTGGGGTCCCACC | |
| | GCGGTTCAGCGGCAGTGGATCTGGGACACAGTTCACT | |
| | CTCGCCATCAGCGGCGTGCAGTGTGACGATGCTGCCA | |
| | CTTACTACTGTCAAGGCAGTTATGATTGTAATAGTGCT | |
| | GATTGTAGTGCTTTCGGCGGAGGGACCGAGGTGGTGG | |
| | TCAAA | |

mAb#170

| | | SEQ ID |
|---|---|---|
| Region | Amino Acid Sequence | NO: |
| HC AbM | GFSLSRYGVI | 35 |
| CDR1 | | |
| HC AbM | YISSTGSTY | 36 |
| CDR2 | | |
| HC AbM | SGLGYDGGRNI | 37 |
| CDR3 | | |
| LC AbM | QSSKSVANSNWLS | 38 |
| CDR1 | | |
| LC AbM | SASTLAS | 39 |
| CDR2 | | |
| LC AbM | AGGYSDISDIFG | 40 |
| CDR3 | | |
| HC Kabat | RYGVI | 41 |
| CDR1 | | |
| HC Kabat | YISSTGSTYYASWAKS | 42 |
| CDR2 | | |
| HC Kabat | SGLGYDGGRNI | 43 |
| CDR3 | | |
| LC Kabat | QSSKSVANSNWLS | 44 |
| CDR1 | | |
| LC Kabat | SASTLAS | 45 |
| CDR2 | | |
| LC Kabat | AGGYSDISDIFG | 46 |
| CDR3 | | |
| HC Chothia | GFSLSRY | 47 |
| CDR1 | | |
| HC Chothia | SSTGS | 48 |
| CDR2 | | |
| HC Chothia | SGLGYDGGRNI | 49 |
| CDR3 | | |
| LC Chothia | QSSKSVANSNWLS | 50 |
| CDR1 | | |
| LC Chothia | SASTLAS | 51 |
| CDR2 | | |
| LC Chothia | AGGYSDISDIFG | 52 |
| CDR3 | | |
| HC IMGT | GFSLSRYG | 53 |
| CDR1 | | |
| HC IMGT | ISSTGST | 54 |
| CDR2 | | |
| HC IMGT | AQSGLGYDGGRNI | 55 |
| CDR3 | | |
| LC IMGT | KSVANSNW | 56 |
| CDR1 | | |
| LC IMGT | SAS | 57 |
| CDR2 | | |
| LC IMGT | AGGYSDISDIFG | 58 |
| CDR3 | | |
| HC | SRYGVI | 59 |
| CONTACT | | |
| CDR1 | | |
| HC | FIGYISSTGSTY | 60 |
| CONTACT | | |
| CDR2 | | |
| HC | AQSGLGYDGGRN | 61 |
| CONTACT | | |
| CDR3 | | |
| LC | ANSNWLSWY | 62 |
| CONTACT | | |
| CDR1 | | |
| LC | LLIYSASTLA | 63 |
| CONTACT | | |
| CDR2 | | |
| LC | AGGYSDISDIF | 64 |
| CONTACT | | |
| CDR3 | | |
| VH | QSMKESEGGLFKPTDTLTLTCTVSGFSLSRYGVIWVRQA | 65 |
| | PGKGLEFIGYISSTGSTYYASWAKSRSTITRNTNENTVTL | |
| | KVTSLTAADTATYFCAQSGLGYDGGRNIWGPGTLVTVS | |
| | S | |
| VL | AAVLTQTPSPVSAAVGGTVSISCQSSKSVANSNWLSWY | 66 |
| | QQKPGQPPKLLIYSASTLASGAPSRFKGSGSGTQFTLTIS | |
| | DVQCDDAATYYCAGGYSDISDIFGFGGGTEVVVK | |
| HC | METGLRWLLLVAVLKGVQCQSMKESEGGLFKPTDTLTL | 67 |
| | TCTVSGFSLSRYGVIWVRQAPGKGLEFIGYISSTGSTYYA | |
| | SWAKSRSTITRNTNENTVTLKVTSLTAADTATYFCAQSG | |
| | LGYDGGRNIWGPGTLVTVSS | |
| LC | MDTRAPTQLLGLLLLWLPGATFAAVLTQTPSPVSAAVG | 68 |
| | GTVSISCQSSKSVANSNWLSWYQQKPGQPPKLLIYSAST | |
| | LASGAPSRFKGSGSGTQFTLTISDVQCDDAATYYCAGG | |
| | YSDISDIFGFGGGTEVVVK | |
| VH* | CAGTCGATGAAGGAGTCCGAGGGAGGTCTCTTCAAGC | 181 |
| | CAACGGATACCCTGACACTCACCTGCACAGTCTCTGG | |
| | ATTCTCCCTCAGTAGGTATGGAGTGATCTGGGTCCGC | |
| | CAGGCTCCAGGGAAGGGGCTGGAATTCATCGGATAC | |
| | ATAAGTAGTACTGGTAGCACATACTACGCGAGCTGGG | |
| | CGAAAAGCCGATCCACCATCACCAGAAACACCAACG | |
| | AGAACACGGTGACTCTGAAAGTGACCAGTCTGACAGC | |
| | CGCGGACACGGCCACCTATTTCTGTGCGCAGAGTGGT | |
| | CTTGGTTATGATGGTGGTAGGAACATCTGGGGCCCAG | |
| | GCACCCTGGTCACCGTCTCCTCG | |
| VL* | GCCGCCGTGCTGACCCAGACTCCATCTCCCGTGTCTG | 182 |
| | CAGCTGTGGGAGGCACAGTCAGCATCAGTTGCCAGTC | |
| | CAGTAAGAGTGTTGCTAATAGCAACTGGCTATCCTGG | |
| | TATCAGCAGAAACCAGGGCAGCCTCCCAAGCTCCTGA | |
| | TCTATTCTGCATCCACTCTGGCATCTGGGGCCCCATCG | |
| | CGGTTCAAAGGCAGTGGATCTGGGACACAGTTCACTC | |
| | TCACCATCAGCGACGTGCAGTGTGACGATGCTGCCAC | |
| | TTACTACTGTGCAGGCGGTTATAGTGATATTAGTGAT | |
| | ATATTTGGTTTCGGCGGAGGGACCGAGGTGGTGGTCA | |
| | AA | |

mAb#21

| | | SEQ ID |
|---|---|---|
| Region | Amino Acid Sequence | NO: |
| HC AbM | GIDLSSNTVS | 69 |
| CDR1 | | |
| HC AbM | VSGYSGRPY | 70 |
| CDR2 | | |
| HC AbM | GSVNVNI | 71 |
| CDR3 | | |
| LC AbM | QSSQSVYSSNRLS | 72 |
| CDR1 | | |
| LC AbM | YTSTLAS | 73 |
| CDR2 | | |
| LC AbM | LGGYDCDIADCSA | 74 |
| CDR3 | | |
| HC Kabat | SNTVS | 75 |
| CDR1 | | |
| HC Kabat | VSGYSGRPYYASWAKG | 76 |
| CDR2 | | |
| HC Kabat | GSVNVNI | 77 |
| CDR3 | | |
| LC Kabat | QSSQSVYSSNRLS | 78 |
| CDR1 | | |
| LC Kabat | YTSTLAS | 79 |
| CDR2 | | |
| LC Kabat | LGGYDCDIADCSA | 80 |
| CDR3 | | |
| HC Chothia | GIDLSSN | 81 |
| CDR1 | | |
| HC Chothia | GYSGR | 82 |
| CDR2 | | |
| HC Chothia | GSVNVNI | 83 |
| CDR3 | | |
| LC Chothia | QSSQSVYSSNRLS | 84 |
| CDR1 | | |
| LC Chothia | YTSTLAS | 85 |
| CDR2 | | |
| LC Chothia | LGGYDCDIADCSA | 86 |
| CDR3 | | |
| HC IMGT | GIDLSSNT | 87 |
| CDR1 | | |
| HC IMGT | SGYSGRP | 88 |
| CDR2 | | |
| HC IMGT | ARGSVNVNI | 89 |
| CDR3 | | |
| LC IMGT | QSVYSSNR | 90 |
| CDR1 | | |
| LC IMGT | YTS | 91 |
| CDR2 | | |
| LC IMGT | LGGYDCDIADCSA | 92 |
| CDR3 | | |
| HC | SSNTVS | 93 |
| CONTACT | | |
| CDR1 | | |
| HC | WIGVSGYSGRPY | 94 |
| CONTACT | | |
| CDR2 | | |
| HC | ARGSVNVN | 95 |
| CONTACT | | |
| CDR3 | | |
| LC | YSSNRLSWF | 96 |
| CONTACT | | |
| CDR1 | | |
| LC | LLMYYTSTLA | 97 |
| CONTACT | | |
| CDR2 | | |
| LC | LGGYDCDIADCS | 98 |
| CONTACT | | |
| CDR3 | | |
| VH | QSVEESGGRLVTPGTPLTLTCTVSGIDLSSNTVSWVRQ | 99 |
| | APGKGLEWIGVSGYSGRPYYASWAKGRFTISKTSSTV | |
| | HLKITSPTAEDTATYFCARGSVNVNIWGPGTLVTVSS | |
| VL | AQVLTQTASPVSAAVGGTVTINCQSSQSVYSSNRLSW | 100 |
| | FQQKPGQPPKLLMYYTSTLASGVPSRFKGSGSGTEFT | |
| | LTISGVQCDDAATYYCLGGYDCDIADCSAFGGGTEVVV | |
| | K | |
| HC | METGLRWLLLVAVLKGVQCQSVEESGGRLVTPGTPLTL | 101 |
| | TCTVSGIDLSSNTVSWVRQAPGKGLEWIGVSGYSGRPY | |
| | YASWAKGRFTISKTSSTVHLKITSPTAEDTATYFCARGS | |
| | VNVNIWGPGTLVTVSS | |
| LC | MDTRAPTQLLGLLLLWLPGATFAQVLTQTASPVSAAVG | 102 |
| | GTVTINCQSSQSVYSSNRLSWFQQKPGQPPKLLMYYTST | |
| | LASGVPSRFKGSGSGTEFTLTISGVQCDDAATYYCLGGY | |
| | DCDIADCSAFGGGTEVVVK | |
| VH* | CAGTCGGTGGAGGAGTCCGGGGGTCGCCTGGTCACGC | 183 |
| | CTGGGACGCCCCTGACACTCACCTGCACAGTCTCTGG | |
| | AATCGACCTCAGTAGCAATACAGTGAGTTGGGTCCGC | |
| | CAGGCTCCAGGGAAGGGGCTGGAGTGGATCGGAGTC | |
| | AGTGGTTATAGTGGTCGTCCATACTACGCGAGCTGGG | |
| | CGAAAGGCCGATTCACCATCTCCAAAACCTCGTCCAC | |
| | GGTACATCTGAAGATCACCAGTCCGACAGCCGAGGAC | |
| | ACGGCCACCTATTTCTGTGCCAGAGGTAGCGTGAATG | |
| | TGAATATCTGGGGCCCAGGCACCCTGGTCACCGTCTC | |
| | CTCA | |
| VL* | GCCCAAGTGCTGACCCAGACTGCATCCCCCGTGTCTG | 184 |
| | CGGCTGTTGGAGGCACAGTCACCATCAATTGCCAGTC | |
| | CAGTCAGAGTGTTTATAGTTCAAACCGCTTATCCTGGT | |
| | TTCAGCAGAAACCAGGGCAGCCTCCCAAACTCCTGAT | |
| | GTATTATACGTCCACTCTGGCATCTGGGGTCCCATCGC | |
| | GGTTCAAAGGCAGTGGATCTGGGACAGAGTTCACTCT | |
| | CACCATCAGCGGCGTACAGTGTGACGATGCTGCCACT | |
| | TACTACTGCCTAGGCGGTTATGATTGTGATATTGCTGA | |
| | TTGTAGCGCTTTCGGCGGAGGGACCGAGGTGGTGGTC | |
| | AAA | |

mAb#82

| | | SEQ ID |
|---|---|---|
| Region | Amino Acid Sequence | NO: |
| HC AbM | GIDLSSNTVS | 103 |
| CDR1 | | |
| HC AbM | VIGYSGNGY | 104 |
| CDR2 | | |
| HC AbM | GSVNCNI | 105 |
| CDR3 | | |
| LC AbM | QSSQSVISNNRLS | 106 |
| CDR1 | | |
| LC AbM | YASTLAS | 107 |
| CDR2 | | |
| LC AbM | LGGYDCNSADCST | 108 |
| CDR3 | | |
| HC Kabat | SNTVS | 109 |
| CDR1 | | |
| HC Kabat | VIGYSGNGYYASWAKG | 110 |
| CDR2 | | |
| HC Kabat | GSVNCNI | 111 |
| CDR3 | | |
| LC Kabat | QSSQSVISNNRLS | 112 |
| CDR1 | | |
| LC Kabat | YASTLAS | 113 |
| CDR2 | | |
| LC Kabat | LGGYDCNSADCST | 114 |
| CDR3 | | |
| HC Chothia | GIDLSSN | 115 |
| CDR1 | | |
| HC Chothia | GYSGN | 116 |
| CDR2 | | |
| HC Chothia | GSVNCNI | 117 |
| CDR3 | | |
| LC Chothia | QSSQSVISNNRLS | 118 |
| CDR1 | | |
| LC Chothia | YASTLAS | 119 |
| CDR2 | | |
| LC Chothia | LGGYDCNSADCST | 120 |
| CDR3 | | |
| HC IMGT | GIDLSSNT | 121 |
| CDR1 | | |
| HC IMGT | IGYSGNG | 122 |
| CDR2 | | |
| HC IMGT | ARGSVNCNI | 123 |
| CDR3 | | |
| LC IMGT | QSVISNNR | 124 |
| CDR1 | | |
| LC IMGT | YAS | 125 |
| CDR2 | | |
| LC IMGT | LGGYDCNSADCST | 126 |
| CDR3 | | |
| HC | SSNTVS | 127 |
| CONTACT | | |
| CDR1 | | |
| HC | WIGVIGYSGNGY | 128 |
| CONTACT | | |
| CDR2 | | |
| HC | ARGSVNCN | 129 |
| CONTACT | | |
| CDR3 | | |
| LC | ISNNRLSWL | 130 |
| CONTACT | | |
| CDR1 | | |
| LC | LLIYYASTLA | 131 |
| CONTACT | | |
| CDR2 | | |
| LC | LGGYDCNSADCS | 132 |
| CONTACT | | |
| CDR3 | | |
| VH | QSVEESGGRLVTPGTPLTLTCTVSGIDLSSNTVSWVRQ | 133 |
| | APGKGLEWIGVIGYSGNGYYASWAKGRFTISKTSTTVD | |
| | LKMTSPTAEDTATYFCARGSVNCNIWGPGTLVTVSS | |
| VL | AQVLTQTASPVSAAVGGTVTINCQSSQSVISNNRLSWL | 134 |
| | QQKPGQPPKLLIYYASTLASGVPSRFKGSGSGTQFTLTI | |
| | SGVQCDDAATYYCLGGYDCNSADCSTFGGGTEVVVK | |
| HC | METGLRWLLLVAVLKGVQCQSVEESGGRLVTPGTPLTL | 135 |
| | TCTVSGIDLSSNTVSWVRQAPGKGLEWIGVIGYSGNGY | |
| | YASWAKGRFTISKTSTTVDLKMTSPTAEDTATYFCARGS | |
| | VNCNIWGPGTLVTVSS | |
| LC | MDTRAPTQLLGLLLLWLPGATFAQVLTQTASPVSAAVG | 136 |
| | GTVTINCQSSQSVISNNRLSWLQQKPGQPPKLLIYYASTL | |
| | ASGVPSRFKGSGSGTQFTLTISGVQCDDAATYYCLGGY | |
| | DCNSADCSTFGGGTEVVVK | |
| VH* | CAGTCGGTGGAGGAGTCCGGGGGTCGCCTGGTCACGC | 185 |
| | CTGGGACACCCCTGACACTCACCTGCACAGTCTCTGG | |
| | AATCGACCTCAGTAGCAATACAGTGAGCTGGGTCCGC | |
| | CAGGCTCCAGGGAAGGGGCTGGAATGGATCGGAGTC | |
| | ATTGGTTATAGTGGTAATGGATACTATGCGAGCTGGG | |
| | CGAAAGGCCGATTCACCATCTCCAAAACCTCGACCAC | |
| | GGTGGATCTGAAAATGACCAGTCCGACAGCCGAGGA | |
| | CACGGCCACCTATTTCTGTGCCAGAGGTAGCGTTAAT | |
| | TGTAATATCTGGGGCCCAGGCACCCTGGTCACCGTCT | |
| | CCTCA | |
| VL* | GCCCAAGTGCTGACCCAGACTGCATCCCCCGTGTCTG | 186 |
| | CGGCTGTGGGAGGCACAGTCACCATCAATTGCCAGTC | |
| | CAGTCAGAGTGTTATTAGTAACAACCGCTTATCCTGG | |
| | CTTCAGCAGAAACCAGGGCAGCCTCCCAAGCTCCTGA | |
| | TCTATTATGCATCCACTCTGGCATCTGGGGTCCCATCG | |
| | CGGTTCAAAGGCAGTGGATCTGGGACACAATTCACTC | |
| | TCACCATCAGCGGCGTGCAGTGTGACGATGCTGCCAC | |
| | TTACTACTGTCTAGGCGGTTATGATTGTAATAGTGCTG | |
| | ATTGTAGCACTTTCGGCGGAGGGACCGAGGTGGTGGT | |
| | CAAA | |

TABLE 3
Protein sequences of tagged full-length wild type (SEQ ID NO 137) and tagged full-length
mutant CRTC3 (SEQ ID NO 138-139) as well as the peptide sequences corresponding to the FLAG
(SEQ ID NO 140) and AVI tags (SEQ ID NO 141) and the 12mer unphosphorylated and
phosphorlyated immunogenic peptides for CRTC3 serine 329 (SEQ ID NOs: 142-143) and serine 370
(SEQ ID NOs: 144-145). All recombinant CRTC3 full-length protein versions are preceded with
N-terminal FLAG and AVI-tags followed by a single glycine residue. The first amino acid of
the non-tag portions of the CRTC3 full-length proteins (SEQ ID NO 137-139) are printed in
bold. The internally produced SIK2 variant for phosphorylation of CRTC3 was expressed as
the construct shown in SEQ ID NO; 187, with the bold portion indicating the SIK2 region.

| | | SEQ ID |
|---|---|---|
| Protein | Sequence | NO: |
| CRTC3 wild type | MDYKDDDDKGLNDIFEAQKIEWHEGMAASPGSGSANPRKFSEKIALHT | 137 |
| protein with N- | QRQAEETRAFEQLMTDLTLSRVQFQKLQQLRLTQYHGGSLPNVSQLRSS | |
| terminal FLAG and AVI | ASEFQPSFHQADNVRGTRHHGLVERPSRNRFHPLHRRSGDKPGRQFDG | |
| tags | SAFGANYSSQPLDESWPRQQPPWKDEKHPGFRLTSALNRTNSDSALHT | |
| | SALSTKPQDPYGGGGQSAWPAPYMGFCDGENNGHGEVASFPGPLKEE | |
| | NLLNVPKPLPKQLWETKEIQSLSGRPRSCDVGGGNAFPHNGQNLGLSPF | |
| | LGTLNTGGSLPDLTNLHYSTPLPASLDTTDHHFGSMSVGNSVNNIPAAM | |
| | THLGIRSSSGLQSSRSNPSIQATLNKTVLSSSLNNHPQTSVPNASALHPSL | |
| | RLFSLSNPSLSTTNLSGPSRRRQPPVSPLTLSPGPEAHQGFSRQLSSTSPLA | |
| | PYPTSQMVSSDRSQLSFLPTEAQAQVSPPPPYPAPQELTQPLLQQPRAPE | |
| | APAQQPQAASSLPQSDFQLLPAQGSSLTNFFPDVGFDQQSMRPGPAFP | |
| | QQVPLVQQGSRELQDSFHLRPSPYSNCGSLPNTILPEDSSTSLFKDLNSAL | |
| | AGLPEVSLNVDTPFPLEEELQIEPLSLDGLNMLSDSSMGLLDPSVEETFRA | |
| | DRL | |
| CRTC3 S329A mutant | MDYKDDDDKGLNDIFEAQKIEWHEGMAASPGSGSANPRKFSEKIALHT | 138 |
| protein | QRQAEETRAFEQLMTDLTLSRVQFQKLQQLRLTQYHGGSLPNVSQLRSS | |
| | ASEFQPSFHQADNVRGTRHHGLVERPSRNRFHPLHRRSGDKPGRQFDG | |
| | SAFGANYSSQPLDESWPRQQPPWKDEKHPGFRLTSALNRTNSDSALHT | |
| | SALSTKPQDPYGGGGQSAWPAPYMGFCDGENNGHGEVASFPGPLKEE | |
| | NLLNVPKPLPKQLWETKEIQSLSGRPRSCDVGGGNAFPHNGQNLGLSPF | |
| | LGTLNTGGSLPDLTNLHYSTPLPASLDTTDHHFGSMSVGNSVNNIPAAM | |
| | THLGIRSSSGLQSSRANPSIQATLNKTVLSSSLNNHPQTSVPNASALHPSL | |
| | RLFSLSNPSLSTTNLSGPSRRRQPPVSPLTLSPGPEAHQGFSRQLSSTSPLA | |
| | PYPTSQMVSSDRSQLSFLPTEAQAQVSPPPPYPAPQELTQPLLQQPRAPE | |
| | APAQQPQAASSLPQSDFQLLPAQGSSLTNFFPDVGFDQQSMRPGPAFP | |
| | QQVPLVQQGSRELQDSFHLRPSPYSNCGSLPNTILPEDSSTSLFKDLNSAL | |
| | AGLPEVSLNVDTPFPLEEELQIEPLSLDGLNMLSDSSMGLLDPSVEETFRA | |
| | DRL | |
| CRTC3 S370A mutant | MDYKDDDDKGLNDIFEAQKIEWHEGMAASPGSGSANPRKFSEKIALHT | 139 |
| protein | QRQAEETRAFEQLMTDLTLSRVQFQKLQQLRLTQYHGGSLPNVSQLRSS | |
| | ASEFQPSFHQADNVRGTRHHGLVERPSRNRFHPLHRRSGDKPGRQFDG | |
| | SAFGANYSSQPLDESWPRQQPPWKDEKHPGFRLTSALNRTNSDSALHT | |
| | SALSTKPQDPYGGGGQSAWPAPYMGFCDGENNGHGEVASFPGPLKEE | |
| | NLLNVPKPLPKQLWETKEIQSLSGRPRSCDVGGGNAFPHNGQNLGLSPF | |
| | LGTLNTGGSLPDLTNLHYSTPLPASLDTTDHHFGSMSVGNSVNNIPAAM | |
| | THLGIRSSSGLQSSRSNPSIQATLNKTVLSSSLNNHPQTSVPNASALHPSL | |
| | RLFSLANPSLSTTNLSGPSRRRQPPVSPLTLSPGPEAHQGFSRQLSSTSPLA | |
| | PYPTSQMVSSDRSQLSFLPTEAQAQVSPPPPYPAPQELTQPLLQQPRAPE | |
| | APAQQPQAASSLPQSDFQLLPAQGSSLTNFFPDVGFDQQSMRPGPAFP | |
| | QQVPLVQQGSRELQDSFHLRPSPYSNCGSLPNTILPEDSSTSLFKDLNSAL | |
| | AGLPEVSLNVDTPFPLEEELQIEPLSLDGLNMLSDSSMGLLDPSVEETFRA | |
| | DRL | |
| FLAG tag protein | DYKDDDDK | 140 |
| AVI tag protein | GLNDIFEAQKIEWHE | 141 |
| CRTC3 S329 peptide | GLQSSRSNPSIQ | 142 |
| CRTC3 pS329 peptide | GLQSSRpSNPSIQ | 143 |
| CRTC3 S370 peptide | RLFSLSNPSLST | 144 |
| CRTC3 pS370 peptide | RLFSLpSNPSLST | 145 |
| SIK2 Variant | MKHHHHHHPMKIEEGKLVIWINGDKGYNGLAEVGKKFEKDTGIKVTVE | 187 |
| | HPDKLEEKFPQVAATGDGPDIIFWAHDRFGGYAQSGLLAEITPDKAFQD | |
| | KLYPFTWDAVRYNGKLIAYPIAVEALSLIYNKDLLPNPPKTWEEIPALDKEL | |
| | KAKGKSALMFNLQEPYFTWPLIAADGGYAFKYENGKYDIKDVGVDNAG | |
| | AKAGLTFLVDLIKNKHMNADTDYSIAEAAFNKGETAMTINGPWAWSNI | |
| | DTSKVNYGVTVLPTFKGQPSKPFVGVLSAGINAASPNKELAKEFLENYLLT | |
| | DEGLEAVNKDKPLGAVALKSYEEELAKDPRIAATMENAQKGEIMPNIPQ | |
| | MSAFWYAVRTAVINAASGRQTVDEALKDAQTPGGSGENLYFQSGSMA | |
| | DGPRHLQRGPVRVGFYDIEGTLGKGNFAVVKLGRHRITKTEVAIKIIDKS | |
| | QLDAVNLEKIYREVQIMKMLDHPHIIKLYQVMETKSMLYLVTEYAKNG | |
| | EIFDYLANHGRLNESEARRKFWQILSAVDYCHGRKIVHRDLKAENLLLD | |
| | NNMNIKIADFGFGNFFKSGELLATWCGSPPYAAPEVFEGQQYEGPQL | |
| | DIWSMGVVLYVLVCGALPFDGPTLPILRQRVLEGRFRIPYFMSEDCEHLI | |
| | RRMLVLDPSKRLTIAQIKEHKWMLIEVPVQRPVLYPQEQENEPSIGEFN | |
| | EQVLRLMHSLGIDQQKTIESLQNKSYNHFAAIYFLLVERLKSHRSS | |

TABLE 4
Linker Sequences

| | | SEQ |
|---|---|---|
| Linker ID | Amino Acid Sequence | ID NO: |
| Linker 1 | GGSEGKSSGSGSESKSTGGS | 146 |
| Linker 2 | GGGSGGGS | 147 |
| Linker 3 | GGGSGGGSGGGS | 148 |
| Linker 4 | GGGSGGGSGGGSGGGS | 149 |
| Linker 5 | GGGSGGGSGGGSGGGSGGGS | 150 |
| Linker 6 | GGGGSGGGGSGGGGS | 151 |
| Linker 7 | GGGGSGGGGSGGGGSGGGGS | 152 |
| Linker 8 | GGGGSGGGGSGGGGSGGGGSGGGGS | 153 |
| Linker 9 | GSTSGSGKPGSGEGSTKG | 154 |
| Linker 10 | IRPRAIGGSKPRVA | 155 |
| Linker 11 | GKGGSGKGGSGKGGS | 156 |
| Linker 12 | GGKGSGGKGSGGKGS | 157 |
| Linker 13 | GGGKSGGGKSGGGKS | 158 |
| Linker 14 | GKGKSGKGKSGKGKS | 159 |
| Linker 15 | GGGKSGGKGSGKGGS | 160 |
| Linker 16 | GKPGSGKPGSGKPGS | 161 |
| Linker 17 | GKPGSGKPGSGKPGSGKPGS | 162 |
| Linker 18 | GKGKSGKGKSGKGKSGKGKS | 163 |
| Linker 19 | STAGDTHLGGEDFD | 164 |
| Linker 20 | GEGGSGEGGSGEGGS | 165 |
| Linker 21 | GGEGSGGEGSGGEGS | 166 |
| Linker 22 | GEGESGEGESGEGES | 167 |
| Linker 23 | GGGESGGEGSGEGGS | 168 |
| Linker 24 | GEGESGEGESGEGESGEGES | 169 |
| Linker 25 | GSTSGSGKPGSGEGSTKG | 170 |
| Linker 26 | PRGASKSGSASQTGSAPGS | 171 |
| Linker 27 | GTAAAGAGAAGGAAAGAAG | 172 |
| Linker 28 | GTSGSSGSGSGGSGSGGGG | 173 |
| Linker 29 | GKPGSGKPGSGKPGSGKPGS | 174 |
| Linker 30 | GSGS | 175 |
| Linker 31 | APAPAPAPAP | 176 |
| Linker 32 | APAPAPAPAPAPAPAPAPAP | 177 |
| Linker 33 | AEAAAKEAAAKEAAAAKEAAAAKEAAAAKAAA | 178 |

We claim:

1. An isolated protein comprising an antigen binding domain that binds phosphorylated CREB Regulated Transcription Coactivator 3 (CRTC3), wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

2. The isolated protein of claim 1, wherein said antigen binding domain binds phosphorylated serine 370 (pSer370) of CRTC3.

3. The isolated protein of claim 1 or 2, wherein said antigen binding domain comprises at least one set of CDR sequences selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively; and
h) a LCDR1, a LCDR2 and a LCDR3, as set forth in SEQ ID NO:28, SEQ ID NO:29, and SEQ ID NO:30 respectively.

4. The isolated protein of claim 3, wherein said antigen binding domain comprises a heavy chain variable region (VH) and light chain variable region (VL), and wherein the VH comprises an amino acid sequence at least 95% identical to the amino acid sequence of SEQ ID NO: 31, and the VL comprises an amino acid sequence at least 95% identical to the amino acid sequence of SEQ ID NO: 32.

5. The isolated protein of claim 4, wherein said antigen binding domain comprises a heavy chain (HC) and light chain (LC), and wherein the HC comprises an amino acid sequence at least 95% identical to the amino acid sequence of SEQ ID NO: 33, and the LC comprises an amino acid sequence at least 95% identical to the amino acid sequence of SEQ ID NO: 34.

6. The isolated protein of claim 1 or 2, wherein said antigen binding domain comprises at least one set of CDR sequences selected from the group consisting of:
a) a HCDR1, a HCDR2, and a HCDR3 as set forth in SEQ ID NO:35, SEQ ID NO:36, and SEQ ID NO:37 respectively;
b) a HCDR1, a HCDR2, and a HCDR3 as set forth in SEQ ID NO:41, SEQ ID NO:42, and SEQ ID NO:43 respectively;
c) a HCDR1, a HCDR2, and a HCDR3 as set forth in SEQ ID NO:47, SEQ ID NO:48, and SEQ ID NO:49 respectively;
d) a HCDR1, a HCDR2, and a HCDR3 as set forth in SEQ ID NO:53, SEQ ID NO:54, and SEQ ID NO:55 respectively;
e) a HCDR1, a HCDR2, and a HCDR3 as set forth in SEQ ID NO:59, SEQ ID NO:60, and SEQ ID NO:61 respectively;
f) a LCDR1, a LCDR2 and a LCDR3, as set forth in SEQ ID NO:38, SEQ ID NO:39, and SEQ ID NO:40 respectively;
g) a LCDR1, a LCDR2 and a LCDR3, as set forth in SEQ ID NO:56, SEQ ID NO:57, and SEQ ID NO:58 respectively; and
h) a LCDR1, a LCDR2 and a LCDR3, as set forth in SEQ ID NO:62, SEQ ID NO:63, and SEQ ID NO:64 respectively.

7. The isolated protein of claim 6, wherein said antigen binding domain comprises a heavy chain variable region (VH) and light chain variable region (VL), and wherein the VH comprises an amino acid sequence at least 95% identical to the amino acid sequence of SEQ ID NO: 65, and the VL comprises an amino acid sequence at least 95% identical to the amino acid sequence of SEQ ID NO: 66.

8. The isolated protein of claim 7, wherein said antigen binding domain comprises a heavy chain (HC) and light chain (LC), and wherein the HC comprises an amino acid sequence at least 95% identical to the amino acid sequence of SEQ ID NO: 67, and the LC comprises an amino acid sequence at least 95% identical to the amino acid sequence of SEQ ID NO: 68.

9. The isolated protein of claim 1, wherein said antigen binding domain binds phosphorylated serine 329 (pSer329) of CRTC3.

10. The isolated protein of claim 1 or 9, wherein said antigen binding domain comprises at least one set of CDR sequences selected from the group consisting of:
a) a HCDR1, a HCDR2, and a HCDR3 as set forth in SEQ ID NO:69, SEQ ID NO:70, and SEQ ID NO:71 respectively;
b) a HCDR1, a HCDR2, and a HCDR3 as set forth in SEQ ID NO:75, SEQ ID NO:76, and SEQ ID NO:77 respectively;
c) a HCDR1, a HCDR2, and a HCDR3 as set forth in SEQ ID NO:81, SEQ ID NO:82, and SEQ ID NO:83 respectively;
d) a HCDR1, a HCDR2, and a HCDR3 as set forth in SEQ ID NO:87, SEQ ID NO:88, and SEQ ID NO:89 respectively;
e) a HCDR1, a HCDR2, and a HCDR3 as set forth in SEQ ID NO:93, SEQ ID NO:94, and SEQ ID NO:95 respectively;
f) a LCDR1, a LCDR2 and a LCDR3, as set forth in SEQ ID NO:72, SEQ ID NO:73, and SEQ ID NO:74 respectively;
g) a LCDR1, a LCDR2 and a LCDR3, as set forth in SEQ ID NO:90, SEQ ID NO:91, and SEQ ID NO:92 respectively; and
h) a LCDR1, a LCDR2 and a LCDR3, as set forth in SEQ ID NO:96, SEQ ID NO:97, and SEQ ID NO:98 respectively.

11. The isolated protein of claim 10, wherein said antigen binding domain comprises a heavy chain variable region (VH) and light chain variable region (VL), and wherein the VH comprises an amino acid sequence at least 95% identical to the amino acid sequence of SEQ ID NO: 99, and the VL comprises an amino acid sequence at least 95% identical to the amino acid sequence of SEQ ID NO: 100.

12. The isolated protein of claim 11, wherein said antigen binding domain comprises a heavy chain (HC) and light chain (LC), and wherein the HC comprises an amino acid sequence at least 95% identical to the amino acid sequence of SEQ ID NO: 101, and the LC comprises an amino acid sequence at least 95% identical to the amino acid sequence of SEQ ID NO: 102.

13. The isolated protein of claim 1 or 9, wherein said antigen binding domain comprises at least one set of CDR sequences selected from the group consisting of:
a) a HCDR1, a HCDR2, and a HCDR3 as set forth in SEQ ID NO:103, SEQ ID NO: 104, and SEQ ID NO: 105 respectively;
b) a HCDR1, a HCDR2, and a HCDR3 as set forth in SEQ ID NO:109, SEQ ID NO:110, and SEQ ID NO: 111 respectively;
c) a HCDR1, a HCDR2, and a HCDR3 as set forth in SEQ ID NO:115, SEQ ID NO: 116, and SEQ ID NO: 117 respectively;
d) a HCDR1, a HCDR2, and a HCDR3 as set forth in SEQ ID NO:121, SEQ ID NO: 122, and SEQ ID NO: 123 respectively;
e) a HCDR1, a HCDR2, and a HCDR3 as set forth in SEQ ID NO:127, SEQ ID NO: 128, and SEQ ID NO: 129 respectively;
f) a LCDR1, a LCDR2 and a LCDR3, as set forth in SEQ ID NO:106, SEQ ID NO: 107, and SEQ ID NO: 108 respectively;
g) a LCDR1, a LCDR2 and a LCDR3, as set forth in SEQ ID NO:124, SEQ ID NO: 125, and SEQ ID NO: 126 respectively; and
h) a LCDR1, a LCDR2 and a LCDR3, as set forth in SEQ ID NO:130, SEQ ID NO:131, and SEQ ID NO: 132 respectively.

14. The isolated protein of claim 13, wherein said antigen binding domain comprises a heavy chain variable region (VH) and light chain variable region (VL), and wherein the VH comprises an amino acid sequence at least 95% identical to the amino acid sequence of SEQ ID NO: 133, and the VL comprises an amino acid sequence at least 95% identical to the amino acid sequence of SEQ ID NO: 134.

15. The isolated protein of claim 14, wherein said antigen binding domain comprises a heavy chain (HC) and light chain (LC), and wherein the HC comprises an amino acid sequence at least 95% identical to the amino acid sequence of SEQ ID NO: 135, and the LC comprises an amino acid sequence at least 95% identical to the amino acid sequence of SEQ ID NO: 136.

16. A pharmaceutical composition comprising the isolated protein of any one of claims 1-15 and a pharmaceutically acceptable carrier.

17. A polynucleotide encoding the isolated protein of any one of claims 1-15.

18. A vector comprising the polynucleotide of claim 17.

19. A host cell comprising the vector of claim 18.

20. A method of producing the isolated protein of any one of claims 1-15, comprising culturing the host cell of claim 19 in conditions the promote expression of the protein, and isolating the protein produced by the host cell.

21. A method of administering an isolated protein to a subject, the method comprising administering a pharmaceutical composition comprising an isolated protein, wherein said isolated protein comprises an antigen binding domain that binds phosphorylated CREB Regulated Transcription Coactivator 3 (CRTC3), wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

22. The method of claim 21, wherein said pharmaceutical composition further comprises a pharmaceutically acceptable carrier.

23. The method of claim 21 or 22, wherein said subject has one or more diseases or disorders.

24. The method of any one of claims 21-23, wherein said pharmaceutical composition is administered in a therapeutically effective amount to treat or prevent the one or more diseases or disorders.

25. The method of any one of claims 21-24, wherein said one or more diseases or disorders comprises one or more selected from the group consisting of: Addison's disease, an ankylosing spondylitis, an atherosclerosis, an autoimmune hepatitis, an autoimmune diabetes, a primary biliary cholangitis, Graves' disease, Guillain-Barre syndrome, Hashimoto's disease, an idiopathic thrombocytopenia, an inflammatory bowel disease (IBD), a systemic lupus erythematosus, a multiple sclerosis, a myasthenia gravis, a psoriasis, an arthritis, a scleroderma, Sjogren's syndrome, a systemic sclerosis, a transplantation, a kidney transplantation, a skin transplantation, a bone marrow transplantation, a graft versus host disease (GVHD), a type I diabetes, a rheumatoid arthritis, a juvenile arthritis, a psoriatic arthritis, Reiter's syndrome, a gouty arthritis, Celiac Disease, Crohn's disease and an ulcerative colitis.

26. A method of differentiating between phosphorylated and unphosphorylated CRTC3 in a sample, comprising:
a) obtaining the sample;
b) contacting the sample with an isolated protein comprising an antigen binding domain that binds phosphorylated CRTC3; and
c) detecting the presence or absence of bound phosphorylated CRTC3, thereby determining that the CRTC3 in the sample is phosphorylated or unphosphorylated.

27. The method of claim 26, wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

28. A method of detecting phosphorylated CRTC3 in a sample, comprising:
a) obtaining the sample;
b) contacting the sample with an isolated protein comprising an antigen binding domain that binds phosphorylated CRTC3; and
c) detecting the bound phosphorylated CRTC3 in the sample, thereby determining the level of phosphorylated CRTC3.

29. The method of claim 28, further comprising:
a) contacting the sample with an isolated protein comprising an antigen binding domain that binds to total CRTC3; and
b) detecting the bound total CRTC3 in the sample, thereby determining the level of total CRTC3.

30. The method of claim 29, further comprising: dividing the level of phosphorylated CRTC3 by the level of total CRTC3, thereby determining the proportion of phosphorylated CRTC3 in the sample.

31. The method of claim 28, wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

32. A method of screening one or more agents that modulates the phosphorylation state of CRTC3, comprising:
a) obtaining a composition, or one or more cells comprising CRTC3 and one or more proteins that modulates the phosphorylation state of CRTC3;
b) contacting the composition or the one or more cell with one or more agents; and
c) detecting the phosphorylation state of CRTC3 according to the methods of claim 6.

33. The method of claim 32, wherein said one or more agents modulates the activity of one or more proteins that modulates the phosphorylation state of CRTC3.

34. The method of claim 33, wherein said one or more proteins increases the phosphorylation of CRTC3.

35. The method of claim 34, wherein said one or more proteins that increases the phosphorylation of CRTC3 comprises a kinase.

36. The method of claim 33, wherein said one or more proteins decreases the phosphorylation of CRTC3.

37. The method of claim 36, wherein said one or more proteins that decreases the phosphorylation of CRTC3 comprises a phosphatase.

38. The method of claim 32, further comprising comparing the level of bound phosphorylated CRTC3 of the composition or cell with the level of bound phosphorylated CRTC3 of a comparator, wherein:
a) an increase in phosphorylation relative to said comparator indicates that said one or more agents is an enhancer of CRTC3 phosphorylation; and
b) a decrease in phosphorylation relative to said comparator indicates that said one or more agents is an inhibitor of CRTC3 phosphorylation.

39. The method of claim 32, wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

40. A method of determining the effect of one or more therapeutics that modulates the level of phosphorylation of CRTC3 in a subject, comprising:
a) administering the one or more therapeutics to the subject;
b) obtaining a sample from the subject;
c) contacting the sample with the antigen binding domain that binds phosphorylated CRTC3; and
d) detecting the level of bound phosphorylated CRTC3 in the sample of the subject as compared to the level of bound phosphorylated CRTC3 of a comparator.

41. The method of claim 40, further wherein an elevated level of bound phosphorylated CRTC3 in the sample of the subject as compared to the level of bound phosphorylated CRTC3 of a comparator indicates one or more selected from the group consisting of: 1) that said one or more therapeutics is efficacious for elevating the level of phosphorylated CTRC3 in a subject and 2) that the concentration of the one or more therapeutics is therapeutically effective.

42. The method of claim 41, further wherein no detection of an elevated level of bound phosphorylated CRTC3 in the sample of the subject as compared to the level of bound phosphorylated CRTC3 of a comparator indicates one or more selected from the group consisting of: 1) that said one or more therapeutics is not efficacious for elevating the level of phosphorylated CTRC3 in a subject and 2) that the concentration of the one or more therapeutics is not therapeutically effective; and the subject is administered one or more selected from the group consisting of: 1) one or more additional therapeutics that elevates the level of phosphorylation of CRTC3 and 2) an increased concentration of the one or more therapeutics.

43. The method of claim 40, further wherein a depressed level of bound phosphorylated CRTC3 in the sample of the subject as compared to the level of bound phosphorylated CRTC3 of a comparator indicates one or more selected from the group consisting of: 1) that said one or more therapeutics is efficacious for depressing the level of phosphorylated CTRC3 in a subject and 2) that the concentration of the one or more therapeutics is therapeutically effective.

44. The method of claim 43, further wherein no detection of a depressed level of bound phosphorylated CRTC3 in the sample of the subject as compared to the level of bound phosphorylated CRTC3 of a comparator indicates one or more selected from the group consisting of: 1) that said one or more therapeutics is not efficacious for depressing the level of phosphorylated CTRC3 in a subject and 2) that the concentration of the one or more therapeutics is not therapeutically effective; and the subject is administered one or more selected from the group consisting of: 1) one or more additional therapeutics that depresses the level of phosphorylation of CRTC3 and 2) an increased concentration of the one or more therapeutics.

45. The method of claim 40, wherein the antigen binding domain that binds phosphorylated CRTC3 comprises at least one set of complementarity determining regions (CDRs) selected from the group consisting of:
a) a heavy chain complementarity determining region (HCDR) 1, a HCDR2, and a HCDR3 as set forth in SEQ ID NO:1, SEQ ID NO:2 and SEQ ID NO:3 respectively;
b) a HCDR1, a HCDR2, and a HCDR3 as set forth in SEQ ID NO:7, SEQ ID NO:8, and SEQ ID NO:9 respectively;
c) a HCDR1, a HCDR2, and a HCDR3 as set forth in SEQ ID NO:13, SEQ ID NO:14, and SEQ ID NO:15 respectively;
d) a HCDR1, a HCDR2, and a HCDR3 as set forth in SEQ ID NO:19, SEQ ID NO:20, and SEQ ID NO:21 respectively;
e) a HCDR1, a HCDR2, and a HCDR3 as set forth in SEQ ID NO:25, SEQ ID NO:26, and SEQ ID NO:27 respectively;
f) a light chain complementarity determining region (LCDR) 1, a LCDR2 and a LCDR3, as set forth in SEQ ID NO:4, SEQ ID NO:5, and SEQ ID NO:6 respectively;
g) a LCDR1, a LCDR2 and a LCDR3, as set forth in SEQ ID NO:22, SEQ ID NO:23, and SEQ ID NO:24 respectively;
h) a LCDR1, a LCDR2 and a LCDR3, as set forth in SEQ ID NO:28, SEQ ID NO:29, and SEQ ID NO:30 respectively;
i) a HCDR1, a HCDR2, and a HCDR3 as set forth in SEQ ID NO:35, SEQ ID NO:36, and SEQ ID NO:37 respectively;
j) a HCDR1, a HCDR2, and a HCDR3 as set forth in SEQ ID NO:41, SEQ ID NO:42, and SEQ ID NO:43 respectively;
k) a HCDR1, a HCDR2, and a HCDR3 as set forth in SEQ ID NO:47, SEQ ID NO:48, and SEQ ID NO:49 respectively;
l) a HCDR1, a HCDR2, and a HCDR3 as set forth in SEQ ID NO:53, SEQ ID NO:54, and SEQ ID NO:55 respectively;
m) a HCDR1, a HCDR2, and a HCDR3 as set forth in SEQ ID NO:59, SEQ ID NO:60, and SEQ ID NO:61 respectively;
n) a LCDR1, a LCDR2 and a LCDR3, as set forth in SEQ ID NO:38, SEQ ID NO:39, and SEQ ID NO:40 respectively;
o) a LCDR1, a LCDR2 and a LCDR3, as set forth in SEQ ID NO:56, SEQ ID NO:57, and SEQ ID NO:58 respectively;
p) a LCDR1, a LCDR2 and a LCDR3, as set forth in SEQ ID NO:62, SEQ ID NO:63, and SEQ ID NO:64 respectively;
q) a HCDR1, a HCDR2, and a HCDR3 as set forth in SEQ ID NO:69, SEQ ID NO:70, and SEQ ID NO:71 respectively;
r) a HCDR1, a HCDR2, and a HCDR3 as set forth in SEQ ID NO:75, SEQ ID NO:76, and SEQ ID NO:77 respectively;
s) a HCDR1, a HCDR2, and a HCDR3 as set forth in SEQ ID NO:81, SEQ ID NO:82, and SEQ ID NO:83 respectively;
t) a HCDR1, a HCDR2, and a HCDR3 as set forth in SEQ ID NO:87, SEQ ID NO:88, and SEQ ID NO:89 respectively;
u) a HCDR1, a HCDR2, and a HCDR3 as set forth in SEQ ID NO:93, SEQ ID NO:94, and SEQ ID NO:95 respectively;
v) a LCDR1, a LCDR2 and a LCDR3, as set forth in SEQ ID NO:72, SEQ ID NO:73, and SEQ ID NO:74 respectively;
w) a LCDR1, a LCDR2 and a LCDR3, as set forth in SEQ ID NO:90, SEQ ID NO:91, and SEQ ID NO:92 respectively;
x) a LCDR1, a LCDR2 and a LCDR3, as set forth in SEQ ID NO:96, SEQ ID NO:97, and SEQ ID NO:98 respectively;
y) a HCDR1, a HCDR2, and a HCDR3 as set forth in SEQ ID NO:103, SEQ ID NO: 104, and SEQ ID NO: 105 respectively;
z) a HCDR1, a HCDR2, and a HCDR3 as set forth in SEQ ID NO:109, SEQ ID NO:110, and SEQ ID NO:111 respectively;
aa) a HCDR1, a HCDR2, and a HCDR3 as set forth in SEQ ID NO:115, SEQ ID NO: 116, and SEQ ID NO: 117 respectively;
bb) a HCDR1, a HCDR2, and a HCDR3 as set forth in SEQ ID NO:121, SEQ ID NO: 122, and SEQ ID NO: 123 respectively;
cc) a HCDR1, a HCDR2, and a HCDR3 as set forth in SEQ ID NO:127, SEQ ID NO: 128, and SEQ ID NO: 129 respectively;
dd) a LCDR1, a LCDR2 and a LCDR3, as set forth in SEQ ID NO:106, SEQ ID NO: 107, and SEQ ID NO: 108 respectively;
ee) a LCDR1, a LCDR2 and a LCDR3, as set forth in SEQ ID NO:124, SEQ ID NO: 125, and SEQ ID NO: 126 respectively; and
ff) a LCDR1, a LCDR2 and a LCDR3, as set forth in SEQ ID NO:130, SEQ ID NO:131, and SEQ ID NO: 132 respectively.

46. A method of isolating phosphorylated CRTC3 from a sample, comprising:
a) obtaining the sample;
b) contacting the sample with a composition comprising protein means for binding to phosphorylated CREB Regulated Transcription Coactivator 3 (CRTC3), wherein said protein means is conjugated to a substrate;
c) washing the sample to remove any unbound species; and
d) removing the bound phosphorylated CRTC3 in the sample from the isolated protein that binds phosphorylated CRTC3 of the disclosure, thereby generating an isolated composition of phosphorylated CRTC3.
